(19) United States
(12) Patent Application Publication
(10) Pub. No.: US 2023/0174958 A1
(43) Pub. Date: Jun. 8, 2023

(54) CRISPR-INHIBITION FOR FACIOSCAPULOHUMERAL MUSCULAR DYSTROPHY

(71) Applicant: NEVADA RESEARCH & INNOVATION CORPORATION, Reno, NV (US)

(72) Inventors: Peter L. Jones, Reno, NV (US); Charis L. Himeda, Reno, NV (US)

(21) Appl. No.: 17/919,198

(22) PCT Filed: Apr. 6, 2021
(86) PCT No.: PCT/US2021/025940

§ 371 Date: Oct. 14, 2022

Related U.S. Application Data
(60) Provisional application No. 63/011,476, filed on Apr. 17, 2020.

(51) Int. Cl. C12N 9/22; C12N 15/86; A61P 21/00

(52) U.S. Cl. CPC C12N 9/22; A61P 21/00; C12N 15/86; C12N 2310/20; C12N 2750/14143

(57) ABSTRACT

The disclosure relates to methods and compositions for inhibiting the expression of the DUX4 gene in skeletal muscle cells. In some aspects, the invention includes a CRISPR interference platform that directs an epigenetic regulator to the DUX4 locus. In some aspects, methods described by the disclosure are useful in modulating DUX4 expression for the treatment of facioscapulohumeral muscular dystrophy (FSHD).

### CROSS-REFERENCE TO RELATED APPLICATIONS

This application claims priority under 35 U.S.C. § 119(e) to U.S. Provisional Application No. 63/011,476 filed Apr. 17, 2020, which is incorporated herein by reference in its entirety.

### BACKGROUND OF THE INVENTION

Facioscapulohumeral muscular dystrophy (FSHD) (MIM 158900 and 158901) is the third most common muscular dystrophy in humans, characterized by progressive weakness and atrophy of specific muscle groups. Both forms of the disease are caused by epigenetic dysregulation of the D4Z4 macrosatellite repeat array at chromosome 4q35. FSHD1, the most common form of the disease, is linked to large deletions of chromatin at this array (Wijmenga, et al. (1990) Lancet. 336:651-3; Wijmenga, et al. (1992) Nat Genet. 2: 26-30; van Deutekom, et al. (1993) Hum Mol Genet. 2: 2037-42). FSHD2 is caused by mutations in proteins that maintain epigenetic silencing. Both conditions lead to a similar relaxation of D4Z4 chromatin (Lemmers, et al. (2012) Nat Genet. 44: 1370-4), resulting in the aberrant expression of the DUX4 retrogene in skeletal muscle. While DUX4 resides in every D4Z4 repeat unit in the macrosatellite array, only the full-length DUX4 mRNA (DUX4-fl) encoded by the distal-most repeat is stably expressed, due to the presence of a polyadenylation signal in disease-permissive alleles (Lemmers, et al. (2010) Science. 329: 1650-3; Snider, et al. (2010) PLoS Genet. 6: e1001181). The DUX4-FL protein, in turn, activates a host of genes normally expressed in early development, which cause pathology when mis-expressed in adult skeletal muscle (Campbell, et al. (2018) Hum Mol Genet.; Himeda, et al. (2019) Ann Rev Genomics Hum Genet. 20:265-291).

There is a clear need in the art for new approaches to correct the epigenetic dysregulation in FSHD and to therapeutically reduce DUX4 expression in skeletal muscle cells so as to reduce the severity of the condition. The present invention addresses this need.

### SUMMARY OF THE INVENTION

As described herein, the present invention relates to methods and compositions useful for the treatment of facioscapulohumeral muscular dystrophy (FSHD).

In one aspect, the invention includes a polynucleotide encoding a CRISPR interference (CRISPRi) platform comprising a single guide RNA (sgRNA) and a fusion polypeptide, wherein the fusion polypeptide further comprises a catalytically inactive Cas9 (dCas9 or iCas9) fused to an epigenetic repressor.

In various embodiments, the sgRNA is under control of the U6 promoter.

In various embodiments, the sgRNA targets the DUX4 locus.

In various embodiments, the fusion polypeptide is under control of a skeletal muscle-specific regulatory cassette.

In various embodiments of the above aspect or any other aspect of the invention delineated herein, the catalytically inactive Cas9 is a dSaCas9.

In various embodiments of the above aspect or any other aspect of the invention delineated herein, the epigenetic repressor is selected from the group consisting of HP1α, HP1γ, the chromo shadow domain and C-terminal extension region of HP1α or HP1γ, MeCP2 transcription repression domain (TRD), and SUV39H1 SET domains.

In certain embodiments, the sgRNA comprises SEQ ID NO: 38, 39, 40, 41, 42, or 43.

In certain embodiments, the fusion polypeptide comprises any one of SEQ ID NOs: 1-4.

In certain embodiments, the polynucleotide comprises any one of SEQ ID NOs: 48-55.

10. In another aspect, the invention includes a vector comprising a polynucleotide encoding a CRISPRi platform comprising a sgRNA and a fusion polypeptide, wherein the fusion polypeptide further comprises a catalytically inactive Cas9 (dCas9 or iCas9) fused to an epigenetic repressor.

In certain embodiments, the sgRNA is under control of the U6 promoter.

In certain embodiments, the sgRNA targets the DUX4 locus.

In certain embodiments, the fusion polypeptide is under control of a skeletal muscle-specific regulatory cassette.

In certain embodiments, the catalytically inactive Cas9 is a dSaCas9.

In certain embodiments, the epigenetic repressor is selected from the group consisting of a HP1α, HP1γ, the chromo shadow domain and C-terminal extension region of HP1α or HP1γ, MeCP2 transcription repression domain (TRD), and SUV39H1 SET domains.

In certain embodiments, the sgRNA comprises SEQ ID NO: 38, 39, 40, 41, 42, or 43.

In certain embodiments, the fusion polypeptide comprises any one of SEQ ID NOs: 1-4.

In certain embodiments, the polynucleotide comprises any one of SEQ ID NOs: 48-55.

In certain embodiments, the vector is an adeno-associated viral (AAV) vector.

In certain embodiments, the vector comprises any one of SEQ ID NOs: 48-55.

In another aspect, the invention includes a method of treating facioscapulohumeral muscular dystrophy (FSHD) in a subject in need thereof, the method comprising administering to the subject an effective amount of a repressor of DUX4 gene expression, wherein the repressor decreases DUX4 gene expression in the skeletal muscle cells of the subject, thereby treating the disorder.

In certain embodiments, the DUX4 repressor is polynucleotide comprising a CRISPRi platform comprising a sgRNA and a fusion polypeptide, wherein the fusion polypeptide further comprises a dCas9 fused to an epigenetic repressor.

In certain embodiments, the sgRNA targets the DUX4 locus.

In certain embodiments, the sgRNA comprises a nucleic acid sequence selected from the group consisting of SEQ ID NOs: 38, 39, 40, 41, 42, or 43.

In certain embodiments, the dCas9 is a dSaCas9.

In certain embodiments, the epigenetic repressor is selected from the group consisting of HP1α, HP1γ, the chromo shadow domain and C-terminal extension region of HP1α or HP1γ, MeCP2 transcription repression domain (TRD), and SUV39H1 SET domains.

In certain embodiments, the fusion polypeptide is encoded by a polynucleotide comprising any one of SEQ ID NOs: 1-4.

In certain embodiments, the polynucleotide comprises any one of SEQ ID NOs: 48-55. In certain embodiments, the subject is a mammal.

In certain embodiments, the mammal is a human.

In certain embodiments, the method comprising administering to the subject an effective amount of the vector of any one of the above aspects or any other aspect of the invention delineated herein.

In certain embodiments, the subject is a mammal.

In certain embodiments, the mammal is a human.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1D depict CRISPRi constructs for epigenetic repression of DUX4. FIG. 1A illustrates the original two-vector system: 1) dSpCas9 fused to the KRAB transcriptional repression domain (TRD) under control of a CKM-based regulatory cassette and 2) a DUX4-targeting sgRNA with SpCas9-compatible scaffold under control of the U6 promoter. FIG. 1B depicts an optimized two-vector system: 1) the smaller dSaCas9 ortholog fused to one of four epigenetic repressors (HP1α, HP1γ, the MeCP2 TRD, or the SUV39H1 pre-SET, SET, and post-SET domains) under control of a minimized skeletal muscle regulatory cassette, and 2) a DUX4-targeting sgRNA with SaCas9-compatible scaffold incorporating modifications that remove a putative Pol III terminator and improve assembly with dCas9 (Tabebordar, et al. (2016) Science. 351: 407-11) under control of the U6 promoter. FIG. 1C depicts an optimized single-vector system. The construct contains a mini version of each epigenetic regulator and sgRNA components in four separate therapeutic cassettes. Size of components is not to scale. FIG. 1D shows a schematic diagram of the FSHD locus at chromosome 4q35. Distances are shown relative to the DUX4 MAL start codon (*). For simplicity, only the distal D4Z4 repeat unit of the macrosatellite array is depicted. DUX4 exons 1 and 2 are located within the D4Z4 repeat, and exon 3 lies in the distal subtelomeric sequence. The locations of sgRNA target sequences (#1-6) are indicated. Positions of ChIP amplicons are shown as unlabeled red bars (in order from 5′ to 3′: DUX4 promoter, exon 1, and exon 3).

FIGS. 2A-2D are a series of graphs illustrating that dSaCas9-mediated recruitment of epigenetic repressors to the DUX4 promoter or exon 1 represses DUX4-fl and DUX4-FL targets in FSHD myocytes. FSHD myocytes were subjected to four serial co-infections with lentiviral (LV) supernatants expressing dSaCas9 fused to either: FIG. 2A) the SUV39H1 pre-SET, SET, and post-SET domains (SET), FIG. 2B) the MeCP2 TRD, FIG. 2C) HP1γ, or FIG. 2D) HP1α, with or without LV expressing sgRNAs targeting DUX4 (#1-6) or non-targeting sgRNAs (NT). Cells were harvested ~72 h following the last round of infection. Expression levels of DUX4-fl and DUX4-FL target genes TRIM43 and MBD3L2 were assessed by qRT-PCR. Data are plotted as the mean + SD value of at least four independent experiments, with relative mRNA expression for cells expressing each dCas9-epigenetic regulator alone set to 1. *p<0.05, **p<0.01, ***p<0.001 are from comparing to NT.

FIGS. 3A-3D depict dSaCas9-mediated recruitment of epigenetic repressors to the DUX4 promoter or exon 1 represses DUX4-fl and DUX4-FL targets in FSHD myocytes. FSHD myocytes were subjected to four serial co-infections with lentiviral (LV) supernatants expressing dSaCas9 fused to either: FIG. 3A) the SUV39H1 pre-SET, SET, and post-SET domains (SET), FIG. 3B) the MeCP2 TRD, FIG. 3C) HP1γ, or FIG. 3D) HP1α, with or without LV expressing sgRNAs targeting DUX4 (#1-6) or non-targeting sgRNAs (NT). Cells were harvested ~72 h following the last round of infection. Expression levels of DUX4-fl and DUX4-FL target genes TRIM43 and MBD3L2 were assessed by qRT-PCR. In all panels, each bar represents relative mRNA expression for a single biological replicate, with expression for cells expressing each dCas9-epigenetic regulator alone set to 1.

FIGS. 4A-4B are a pair of graphs illustrating the enzymatic activity of the SET domain is required for DUX4-fl repression. FSHD myocytes were infected as in FIG. 2 with LV supernatants expressing dSaCas9-SET containing a mutation (C326A) within the SET domain that abolishes enzymatic activity (SET-mt) (Rea, et al. (2000) 406: 593-9) with or without LV expressing sgRNAs targeting DUX4 (#1-4) or non-targeting sgRNAs (NT). Expression levels of DUX4-fl were assessed by qRT-PCR. In FIG. 4A, data are plotted as the mean + SD value of four independent experiments, with relative mRNA expression for cells expressing dCas9-SET-mt alone set to 1. In FIG. 4B, each bar represents relative mRNA expression for a single biological replicate, with expression for cells expressing dCas9-SET-mt alone set to 1.

FIGS. 5A-5D are a series of graphs illustrating that the targeting of dSaCas9-epigenetic repressors to DUX4 has no effect on MYH1 or D4Z4 proximal genes. (FIGS. 5A-5D) Expression levels of the terminal muscle differentiation marker Myosin heavy chain 1 (MYH1) and the D4Z4 proximal genes FRG1 and FRG2 were assessed by qRT-PCR in the FSHD myocyte cultures described in FIG. 2. Data are plotted as the mean + SD value of at least four independent experiments, with relative mRNA expression for cells expressing each dCas9-epigenetic regulator alone set to 1.

FIGS. 6A-6D are a series of graphs illustrating that the targeting of dSaCas9-epigenetic repressors to DUX4 has no effect on MYH1 or D4Z4 proximal genes. FIGS. 6A-6D) Expression levels of the terminal muscle differentiation marker Myosin heavy chain 1 (MYH1) and the D4Z4 proximal genes FRG1 and FRG2 were assessed by qRT-PCR in the FSHD myocyte cultures described in FIG. 2. In all panels, each bar represents relative mRNA expression for a single biological replicate, with expression for cells expressing each dCas9-epigenetic regulator alone set to 1.

FIGS. 7A-7B are a pair of graphs demonstrating that the targeting of dSaCas9-epigenetic repressors to DUX4 has no effect on closest-match off-target (OT) genes expressed in skeletal muscle. Levels of Lysosomal amino acid transporter 1 homolog (LAAT1) (FIG. 7A), Ribosome biogenesis regulatory protein homolog (RRS1), or Guanine nucleotide-binding protein G(i) subunit alpha-1 isoform 1 (GNAI1) (FIG. 7B) were assessed by qRT-PCR in the relevant FSHD myocyte cultures described in FIG. 2. Intron 1 of LAAT1 contains a potential OT match to sgRNA #1. The single exon of RRS1 and the downstream flanking sequence of GNAI1 contain potential OT matches to sgRNA #5. Data are plotted as the mean + SD value of at least five independent experiments, with relative mRNA expression for cells expressing each dCas9-epigenetic regulator alone set to 1.

FIGS. 8A-8B demonstrate that the targeting of dSaCas9-epigenetic repressors to DUX4 has no effect on closest-match off-target (OT) genes expressed in skeletal muscle. Levels of Lysosomal amino acid transporter 1 homolog (LAAT1) (FIG. 8A), Ribosome biogenesis regulatory protein homolog (RRS1), or Guanine nucleotide-binding protein G(i) subunit alpha-1 isoform 1 (GNAI1) (FIG. 8B) were assessed by qRT-PCR in the relevant FSHD myocyte cultures described in FIG. 2. Intron 1 of LAAT1 contains a potential OT match to sgRNA #1. The single exon of RRS1 and the downstream flanking sequence of GNAI1 contain potential OT matches to sgRNA #5. In all panels, each bar represents relative mRNA expression for a single biological replicate, with expression for cells expressing each dCas9-epigenetic regulator alone set to 1.

FIGS. 9A-9C are a series of graphs demonstrating that dSaCas9-mediated recruitment of epigenetic repressors to DUX4 increases chromatin repression at the locus. ChIP assays were performed using FSHD myocytes infected with LV supernatants expressing each dSaCas9-epigenetic regulator + sgRNA targeting the DUX4 promoter or exon 1. Chromatin was immunoprecipitated using antibodies specific for HP1α (FIG. 9A) or KAP1 (FIG. 9B) and analyzed by qPCR using primers to the promoter (Pro), transcription start site (TSS), or exon 3 of DUX4 or to MYOD1, or antibodies specific for the elongating form of RNA-Pol II (phospho-serine 2) (FIG. 9C) and analyzed by qPCR using primers specific to DUX4 exon1/intron1 on chromosome 4 or to MYOD1. MYOD1 was used as a negative control for an active gene that should not be affected by CRISPRi targeted to DUX4. Locations of DUX4 primers are shown in FIG. 1D. Data are presented as fold enrichment of the target region by each specific antibody normalized to α-histone H3, with enrichment for mock-infected cells set to 1. For all panels, each bar represents the average of at least three independent ChIP experiments. *p<0.05, **p<0.01, ***p<0.001 are from comparing to enrichment at MYOD1.

FIGS. 10A-10C illustrate that dSaCas9-mediated recruitment of epigenetic repressors to DUX4 increases chromatin repression at the locus. ChIP assays were performed using FSHD myocytes infected with LV supernatants expressing each dSaCas9-epigenetic regulator + sgRNA targeting the DUX4 promoter or exon 1. Chromatin was immunoprecipitated using antibodies specific for HP1α (FIG. 10A) or KAP1 (FIG. 10B) and analyzed by qPCR using primers to the promoter (Pro), transcription start site (TSS), or exon 3 of DUX4 or to MYOD1, or antibodies specific for the elongating form of RNA-Pol II (phospho-serine 2) (FIG. 10C) and analyzed by qPCR using primers specific to DUX4 exon1/intron1 on chromosome 4 or to MYOD1. MYOD1 was used as a negative control for an active gene that should not be affected by CRISPRi targeted to DUX4. Locations of DUX4 primers are shown in FIG. 1D. Data are presented as fold enrichment of the target region by each specific antibody normalized to α-histone H3, with enrichment for mock-infected cells set to 1. In all panels, each bar represents a single biological replicate.

FIG. 11 is a graph depicting PCR detection of AAV genomes in tissues. The presence of AAV genomes in various mCherry-expressing and non-expressing tissues was assessed by qPCR using primers against AAV9 and normalizing to the single copy Rosa26 gene. This confirmed that tissues such as kidney and liver, which did not express any detectable mCherry, were highly transduced, supporting the tissue specificity of the FSHD-optimized expression cassette.

FIGS. 12A-12U are a series of micrographs and a diagram illustrating that the FSHD-optimized regulatory cassette is active in skeletal muscles, but not in cardiac muscle. AAV9 viral particles containing mCherry under control of the FSHD-optimized regulatory cassette (FIG. 12U) were delivered by retro-orbital injection to wild-type mice and the fluorescent signal was visualized at 12 weeks post-injection using Leica MZ9.5/DFC7000T imaging system. For two-tissue panels FIGS. 12A-12L, tissues from uninjected mice are shown on left. Single-tissue panels 12M-12N are uninjected; panels 12O-12T are AAV injected. All injected tissues are indicated by an asterisk. Expression of mCherry was detected in skeletal muscles (tibialis anterior TA, gastrocnemius GA, and quadriceps QUA, as well as diaphragm, pectoral, abdominal, and facial muscles) and was undetectable in the heart.

FIGS. 13A-13T are a series of images demonstrating that the FSHD-optimized regulatory cassette is not active in non-skeletal muscle tissues. Non-muscle tissues from the AAV9 injected wild-type mice assayed in FIG. 12 were similarly assayed for mCherry expression. Panels A, B, K, and L only show tissues from AAV injected mice; the remaining panels show tissues from uninjected mice (left) and injected mice (right and indicated by an asterisk). In panels A and B, the sciatic nerve is indicated by a black arrow.

FIGS. 14A-14F illustrate targeting dSaCas9-repressors to DUX4 has minimal effects on global gene expression in FSHD myocytes (FIGS. 14A-14E). FSHD myocytes were transduced with: (FIG. 14A) dSaCas9-KRAB + sgRNA #6, (FIG. 14B) dSaCas9-HP1α + sgRNA #2, (FIG. 14C) dSaCas9-HP1γ + sgRNA #5, (FIG. 14D) dSaCas9-SET + sgRNA #1, or (FIG. 14E) dSaCas9-TRD + sgRNA #6. For each treatment, five independent experiments were analyzed by RNA-seq using the Illumina HiSeq 2 x 100 bp platform. Adjusted volcano scatterplots show the global transcriptional changes between each treatment versus mock-infected cells. Each data point represents a gene. Upregulated genes (p < 0.05 and a log2 fold change > 1) are indicated by grey dots. Downregulated genes (p < 0.05 and a log2 fold change < -1) are indicated by dark grey dots. Unique differentially expressed genes (summarized in F) are indicated by light grey dots.

FIG. 15 shows gene ontology (GO) analysis of mock vs KRAB.

FIG. 16 shows gene ontology (GO) analysis of mock vs HPlγ.

FIG. 17 shows gene ontology (GO) analysis of mock vs HP1α.

FIG. 18 shows gene ontology (GO) analysis of mock vs SET.

FIG. 19 shows gene ontology (GO) analysis of mock vs TRD.

FIGS. 20A-20F illustrate in vivo targeting of dSaCas9-repressors to DUX4 exon 1 represses DUX4-fl and DUX4-FL targets in ACTA1-MCM;FLExD bi-transgenic mice (FIGS. 20A-20F). dSaCas9-TRD or -KRAB ± sgRNAs were delivered intramuscularly using AAV9 to the ACTA1-MCM;FLExD moderate pathology FSHD-like transgenic mouse model, which carries one human D4Z4 repeat. Expression of DUX4-fl and DUX4-FL downstream markers Wfdc3 and Slc34a2 were assessed by qRT-PCR and normalized to levels of Rpl37. Copy-number ratios of dSaCas9-TRD or -KRAB to sgRNA are indicated. *p < 0.05, **p < 0.01 are from comparing to dSaCas9-TRD or -KRAB control.

FIGS. 21A-21B illustrate that the CRISPRi all-in-one vector effectively represses DUX4-fl and its targets in FSHD1 and FSHD2 myocytes. FSHD1 (FIG. 21A) or FSHD2 (FIG. 21B) primary myocytes were transduced with an all-in-one vector expressing dSaCas9-TRD and a DUX4-targeting sgRNA. Expression levels of DUX4-fl and its target genes TRIM43 and MBD3L2 were assessed by qRT-PCR, as well as MYH1 (which should be unaffected) for comparison. Data are plotted as the mean + SD value of at least three independent experiments, with relative mRNA expression for mock-infected cells set to 1. *p<0.05, **p<0.01, ***p<0.001 are from comparing to mock.

FIG. 22 illustrates that the CRISPRi all-in-one vectors with minimized HP1α and HP1γ effectively repress DUX4-fl and its targets in FSHD1 myocytes. FSHD1 primary myocytes were transduced with an all-in-one vector expressing: 1) dSaCas9 fused to the chromo shadow domain and C-terminal extension of either HP1α or HP1 γ and 2) a DUX4-targeting sgRNA or a non-targeting equivalent (HP1α-NT). Expression levels of DUX4-fl and its target genes TRIM43 and MBD3L2 were assessed by qRT-PCR, as well as MYH1 (which should be unaffected) for comparison. Data are plotted as the mean + SD value of three independent experiments, with relative mRNA expression for mock-infected cells set to 1. *p<0.05, **p<0.01 are from comparing to mock.

FIGS. 23A-23H are a series of micrographs illustrating that the modified FSHD-optimized regulatory cassette displays increased activity in soleus, diaphragm, and heart. mCherry under control of the modified FSHD-optimized regulatory cassette was delivered in AAV9 by RO injection to wild-type mice and the fluorescent signal was visualized at 12 wk post-injection with the same exposure time (300 ms), except where indicated. For two-tissue panels A-G, Injected tissues are marked with *. For panel C, soleus muscles are shown on left and EDL muscles on right. Single-tissue panel H is injected. As with the previous cassette (Himeda, et al. (2020) Mol Ther Methods Clin Dev. 20:298-311), mCherry expression was high in the fast-twitch muscles shown, as well as pectoral, abdominal, and facial muscles (not shown). As an improvement from the previous cassette (Himeda, et al. (2020) Mol Ther Methods Clin Dev. 20:298-311), mCherry expression was detected in soleus (SOL) and increased in diaphragm. While mCherry expression in the heart was also increased, importantly, expression was still undetectable in all non-muscle tissues (gut and liver shown).

FIGS. 24A-24K are a table illustrating the significant DEGs following targeting of dSaCas9-repressors to DUX4.

FIG. 25 is a table illustrating the comparison of DEGs following targeting of dSaCas9-repressors to DUX4.

FIGS. 26A-26B are a table illustrating changes in expression among developmental and myogenic DEGs following targeting of dSaCas9-repressors to DUX4.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In certain embodiments, the cancer is medullary thyroid carcinoma.

The term “cleavage” refers to the breakage of covalent bonds, such as in the backbone of a nucleic acid molecule. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides may be used for targeting cleaved double-stranded DNA.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

A “disease” is a state of health of an animal wherein the animal cannot maintain homeostasis, and wherein if the disease is not ameliorated then the animal’s health continues to deteriorate. In contrast, a “disorder” in an animal is a state of health in which the animal is able to maintain homeostasis, but in which the animal’s state of health is less favorable than it would be in the absence of the disorder. Left untreated, a disorder does not necessarily cause a further decrease in the animal’s state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein’s or peptide’s sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The term “epigenetic” as used herein refers to heritable influences on gene expression that do not involve alterations in DNA nucleotide sequence. Epigenetic regulation can enhance or inhibit expression of affected genes, and can involve chemical modifications of the deoxyribose backbone of the DNA or the association of DNA/histone protein complexes or both.

The term “epigenetic regulator” as used herein refers to factors, enzymes, compounds, or compositions that act to alter the epigenetic status of a specific DNA locus. Epigenetic regulators can induce or catalyze the modification of DNA-associated proteins or the chemical structure of the DNA itself.

The terms “epigenetic tag” or “epigenetic marker” or “epigenetic mark” as used interchangeably herein, describe the specific chemical modifications made to DNA and DNA-associated proteins that result in epigenetic regulation of gene expression. Examples of epigenetic marks or tags can include but are not limited to the addition or removal of methyl or acetyl groups from CpG dinucleotides and histone proteins. The number and density of epigenetic tags or marks may correlate with the degree of epigenetic regulation a particular DNA locus is subject to.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “transgene” refers to the genetic material that has been or is about to be artificially inserted into the genome of an animal, particularly a mammal and more particularly a mammalian cell of a living animal.

The term “transgenic animal” refers to a non-human animal, usually a mammal, having a non-endogenous (i.e., heterologous) nucleic acid sequence present as an extrachromosomal element in a portion of its cells or stably integrated into its germ line DNA (i.e., in the genomic sequence of most or all of its cells), for example a transgenic mouse. A heterologous nucleic acid is introduced into the germ line of such transgenic animals by genetic manipulation of, for example, embryos or embryonic stem cells of the host animal.

The term “knockout mouse” refers to a mouse that has had an existing gene inactivated (i.e. “knocked out”). In some embodiments, the gene is inactivated by homologous recombination. In some embodiments, the gene is inactivated by replacement or disruption with an artificial nucleic acid sequence.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The invention relates to methods and compositions useful for the treatment of facioscapulohumeral muscular dystrophy (FSHD). This disorder results from the incomplete epigenetic silencing of the DUX4 gene locus, leading to the inappropriate and pathogenic expression of the DUX4 gene in skeletal muscle. In some embodiments, expression of DUX4 can be inhibited via the use of epigenetic modulators that alter the chromatin structure of the DUX4 locus, resulting in repressed transcription. In some embodiments, a CRISPR inhibitory (CRISPRi) system is used to direct the epigenetic modulators to the DUX4 locus through the use of specific single guide RNAs (sgRNAs). In some embodiments of the invention, epigenetic modulator proteins are coupled to catalytically-dead Cas9 (dCas9) proteins, which when combined with sequence-specific sgRNAs and controlled by tissue-specific promoters, ensure expression and function of the epigenetic modulators only in skeletal muscle cells.

The present invention provides methods and compositions for the treatment of FSHD in a subject in need thereof. In some embodiments, the method involves administering to the subject a therapeutically effective amount of an epigenetic modulator coupled to a CRISPRi system that targets the DUX4 locus specifically in muscle cells. In some embodiments, the composition has been uniquely modified for size so that it can be packaged within an adeno-associated viral (AAV) vector as a single polynucleotide, thus allowing for in vivo use in a clinical setting.

### CRISPR/Cas9-Based Systems

“Clustered Regularly Interspaced Short Palindromic Repeats” and “CRISPR”, as used herein are terms that refer to a microbial nuclease system that evolved as a defense against invading phages and plasmids and provides a form of acquired immunity for prokaryotic cells. CRISPR loci are flanked by segments of “spacer DNA”, which are short sequences derived from viral genomic material. In the Type II CRISPR system, spacer DNA hybridizes to transactivating RNA (tracrRNA) and is processed into CRISPR-RNA (crRNA), which then associates with CRISPR-associated (Cas) nucleases to form complexes that recognize and degrade foreign DNA. In an embodiment of the invention, CRISPR system utilizes a Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, or other nucleases known in the art, and any combination thereof.

Examples of CRISPR nucleases include, but are not limited to, Cas9 dCas9, Cas6, Cpf1, Cas12a, Cas13a, CasX, CasY, and natural and synthetic variants thereof.

Three classes of CRISPR systems (Types I, II and III effector systems) are known. The Type II effector system carries out targeted DNA double-strand break in four sequential steps, using a single Cas nuclease, Cas9, to cleave dsDNA. Compared to the Type I and Type III effector systems, which require multiple distinct effectors acting as a complex, the relative simplicity of the Type II system enables it’s use in other cell types, such as eukaryotic cells.

CRISPR target recognition occurs upon detection of complementary pairing between a “protospacer” sequence in the target DNA and the spacer sequence in the crRNA. The Cas9 nuclease then cleaves the target DNA if a matched protospacer-adjacent motif (PAM) is also present at the 3′ end of the protospacer. Different Type II systems have differing PAM sequence requirements. In some embodiments, the S. pyogenes CRISPR system may have the PAM sequence for this Cas9 (SpCas9) as 5′-NRG-3′, where R is either A or G, and confers the specificity of this system to human cells. A unique capability of the CRISPR/Cas9 system is the straightforward ability to simultaneously target multiple distinct genomic loci by co-expressing a single Cas9 protein with two or more sgRNAs. For example, the Streptococcus pyogenes ( S. pyogenes) Type II system naturally prefers to use an “NGG” sequence, where “N” can be any nucleotide, but also accepts other PAM sequences, such as “NAG” in engineered systems (Hsu et al, (2013) Nature Biotechnology, 10:1038). Similarly, the Cas9 derived from Neisseria meningitidis (NmCas9) normally has a native PAM of NNNNGATT, but is able to recognize a variety of PAM sequences.

The guide RNA (sgRNA) can include, for example, a nucleotide sequence that comprises an at least 12-20 nucleotide sequence that is complementary to the target DNA sequence and can include a common scaffold RNA sequence at its 3′ end which resembles the tracrRNA sequence or any RNA sequences that function as a tracrRNA. A sgRNA sequence can be determined, for example, by identifying a sgRNA binding site by locating a PAM sequence in the target DNA, and then choosing about 12 to 20 or more nucleotides immediately upstream of the PAM site. The spacer sequence (gap size) between the two sgRNA binding sites on a target DNA can depend on the target DNA sequence and can be determined by those skilled in the art.

In one embodiment of the present invention, introducing the CRISPR system comprises introducing an inducible CRISPR system. The CRISPR system may be induced by exposing the cell comprising the CRISPR vector to an agent that activates an inducible promoter in the CRISPR system, such as the Cas expression vector. In such an embodiment, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. In another embodiment, the CRISPR system may be induced by a tissue-specific promoter. In this case, the promoter from a gene whose expression is largely limited to the cell or tissue type of interest is used to drive expression of the CRISPR vector. Thus, expression of the CRISPR system is restricted to only certain cell types. In one embodiment of the invention, the CRISPR system is under control of a regulatory cassette based on the Creatine Kinase, M-type (CKM) enhancer and promoter, which limits its expression to skeletal muscle cells.

### Inactivated dCas9 CRISPR Systems

CRISPR/Cas9-based systems used in the invention may include a Cas9 protein or a fragment thereof, a Cas9 fusion protein, a nucleic acid encoding a Cas9 protein or a fragment thereof, or a nucleic acid encoding a Cas9 fusion protein. Cas9 protein is an endonuclease that cleaves nucleic acid and is encoded by the CRISPR loci and is involved in the Type II CRISPR system. The Cas9 protein may be from any bacterial or archaea species, such as Streptococcus pyogenes. Cas9 sequences and structures from different species are known in the art, see, e.g., Ferretti et al., Proc Natl Acad Sci USA. (2001); 98(8): 4658-63; Deltcheva et al., Nature. 2011 Mar. 31; 471(7340):602-7; and Jinek et al., Science. (2012); 337(6096):816-21, incorporated herein by reference in their entirety.

S. pyogenes Cas9 is perhaps the most widely-used Cas9 molecule. Notably, S. pyogenes Cas9 is quite large (the gene itself is over 4.1 Kb), making it challenging to be packed into certain delivery vectors. For example, Adeno-associated virus (AAV) vector has a packaging limit of 4.5 or 4.75 Kb. This means that Cas9 as well as regulatory elements such as a promoter and a transcription terminator all have to fit into the same viral vector. Constructs larger than 4.5 or 4.75 Kb will lead to significantly reduced virus production. One possibility is to use a functional fragment of S. pyogenes Cas9. Another possibility is to split Cas9 into its sub-portions (e.g., the N-terminal lobe and the C-terminal lobe of Cas9). Each sub-portion is expressed by a separate vector, and these sub-portions associate to form a functional Cas9. See, e.g., Chew et al., Nat Methods. 2016; 13:868-74; Truong et al., Nucleic Acids Res. 2015; 43: 6450-6458; and Fine et al., Sci Rep. 2015; 5:10777, incorporated by reference herein in their entirety.

Alternatively, shorter Cas9 molecules from other species can be used in the compositions and methods disclosed herein, e.g., Cas9 molecules from Staphylococcus aureus, Campylobacter jejuni, Corynebacterium diphtheria, Eubacterium ventriosum, Streptococcus pasteurianus, Lactobacillus farciminis, Sphaerochaeta globus, Azospirillum (strain B510), Gluconacetobacter diazotrophicus, Neisseria cinerea, Roseburia intestinalis, Parvibaculum lavamentivorans, Nitratifractor salsuginis (strain DSM 16511), Campylobacter lari (strain CF89-12), or Streptococcus thermophilus (strain LMD-9).

In one embodiment of the present invention, the present disclosure is directed to a chimeric fusion protein including a DNA modifying domain fused to a catalytically inactive Cas protein. One who is skilled in the art would recognize that inactivated Cas nucleases are referred to interchangeably as “dead” Cas, iCas, or dCas proteins. In this way, the dCas9 protein lacks normal nuclease activity but retains the sgRNA-binding and DNA-targeting activity of the wildtype protein. dCas9 proteins derived from S. pyogenes (dSpCas9), paired with specific sgRNAs can be targeted to genes in bacteria, yeast, and human cells in order to silence gene expression either through steric hindrance or by fusion with other gene expression-modifying proteins. Such CRISPR systems that reduce or interfere with transcription of the target gene are known as CRISPR interference or CRISPRi or sgRNA/CRISPRi systems.

Suitable dCas molecules for the CRISPRi system of certain embodiments of the invention can be derived from a wild type Cas molecules, and can be from a type I, type II, or type III CRISPR-Cas systems. In some embodiments, suitable dCas molecules can be derived from a Cas1, Cas2, Cas3, Cas4, Cas5, Cash, Cas7, Cas8, Cas9, or Cas10 molecule. In some embodiments of the invention, the dCas molecule is derived from a Cas9 molecule. The dCas9 molecule can be obtained, for example, by introducing point mutations (e.g., substitutions, deletions, or additions) in the Cas9 molecule at the DNA-cleavage domain, e.g., the nuclease domain, e.g., the RuvC and/or HNH domain. See, e.g., Jinek et al., Science (2012) 337:816-21. Similar mutations can also apply to any other Cas9 proteins from any other nature sources and from any artificially mutated Cas9 proteins from any other species such as, for example, Streptococcus thermophiles, Streptococcus salivarius, Streptococcus pasteurianus, Streptococcus mutans, Streptococcus mitis, Streptococcus infantarius, Streptococcus intermedius, Streptococcus equ, Streptococcus agalactiae, Streptococcus anginosus, Bacillus thuringiensis. Finitimus, Streptococcus dysgalactiae, Streptococcus gallolyticus, Streptococcus macedonicus, Streptococcus gordonii, Streptococcus suis, Streptococcus iniae, Neisseria meningitides, Lactobacillus casei, Lactobacillus salivarius, Listeria innocua, Listeria monocytogenes, Lactobacillus buchneri, Lactobacillus paracasei, Lactobacillus sanfranciscensis, Lactobacillus fermentum, Listeria innocua serovar, Lactobacillus rhamnosus, Lactobacillus casei, Lactobacillus sanfranciscensis, Haemophilus sputorum, Geobacillus, Enterococcus hirae, Enterococcus faecalis, Bacillus cereus, Treponema socranskii, Finegoldia magna and others. Similar catalytically inactive mutations can also apply to any other Cas9 proteins from any other natural sources, from any artificially mutated Cas9 proteins, and/or from any artificially created protein fragments that comprise a dCas9 like sgRNA binding domain.

### dCas9 Fusion Proteins

In one embodiment of the invention, the CRISPR/dCas9-based system may include a fusion protein. The fusion protein may comprise a catalytically inactive Cas (dCas) protein conjugated to a second polypeptide via a short linker polypeptide sequence. In some embodiments of the invention, the second polypeptide comprises a DNA modifying domain derived from any DNA modification enzyme known to those skilled in the art. The DNA modifying domain of the fusion protein can be a full-length DNA modifying enzyme or a domain obtained from the full-length DNA modifying enzyme in which the domain retains the DNA modifying activity of the full-length DNA modifying enzyme.

In some embodiments of the invention, the second polypeptide is an enzyme or a functional domain from an enzyme having an activity selected from the group consisting of, but not limited to, transcription activation, transcription repression, transcription release factor activity, histone modification activity, epigenetic transcriptional repression activity, nuclease activity, nucleic acid association activity, methylase activity, and demethylase activity among others.

In one embodiment of the invention, the second polypeptide domain may have an epigenetic repressor activity. The epigenetic repressor activity can include a number of mechanisms affect transcriptional gene activity by inducing structural changes of the chromatin. Examples of such mechanisms include, but are not limited to, DNA methylation and demethylation as well as histone modifications including deacetylation, acetylation, methylation, and demethylation. In some embodiments of the invention, the dCas9 fusion protein comprises an epigenetic repressor derived from the SUV39H1 pre-SET, SET, and post-SET domains. SUV39H1 is a histone methyltransferase that trimethylates lysine 9 of histone H3, a repressive mark that recruits other repressive factors such as HP1 and results in transcriptional silencing. All three SET domains are necessary for methyltransferase activity. In some embodiments of the invention, the dCas9 protein is fused an epigenetic regulator derived from an HP1 family protein. HP1 or heterochromatin protein 1 proteins bind to methylated histone H3 and help form heterochromatin complexes that repress transcriptional activity. In some embodiments of the invention, the HP1 protein is HP1α, which normally localizes to heterochromatin. In some embodiments of the invention, the HP1 protein is HP1γ, which similarly localizes to heterochromatin and mediates transcriptional silencing. In some embodiments of the invention, the dCas9 protein is fused to the chromo shadow domain and C-terminal extension regions of HP1α or HP1γ. HP1γ is particularly enriched in the normal D4Z4 macrosatellite array that serves to silence the DUX4 gene in healthy skeletal muscle cells and HP1γ binding is lost in FSHD. In some embodiments of the invention, the dCas9 fusion protein comprises a transcriptional repressor domain (TRD) derived from MeCP2. This domain specifically binds repressive histone marks and forms co-repressor complexes with other regulatory proteins to enforce transcriptional silencing.

### Gene Transfer Systems and Adeno-Associated Virus (AAV)

Gene transfer systems, such as those described in the present invention, depend upon a vector or vector system to shuttle the genetic constructs into target cells. Methods of introducing a nucleic acid into the hematopoietic stem or progenitor cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about -20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., (1991) Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362. Currently, the most efficient and effective way to accomplish the transfer of genetic constructs into living cells is through the use of vector systems based on viruses that have been made replication-defective. Some of the most effective vectors known in the art are those based on adeno-associated viruses (AAVs). AAVs are small viruses of the parvoviridae family that make attractive vectors for gene transfer in that they are replication defective, not known to cause any human disease, cause only a very mild immune response, can infect both actively dividing and quiescent cells, and stably persist in an extrachromosomal state without integrating into the target cell’s genome. In certain embodiments, the present disclosure provides an AAV vector comprising the dCas9-based CRISPRi system of the invention.

Regardless of the method used to introduce the nucleic acid into the cell, a variety of assays may be performed to confirm the presence of the nucleic acid in the cell. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Pharmaceutical Compositions

Pharmaceutical compositions of the present invention may comprise as described herein, in combination with one or more pharmaceutically or physiologically acceptable carriers, diluents, adjuvants or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient’s disease, although appropriate dosages may be determined by clinical trials.

Pharmaceutical compositions of the present invention may be administered in solid or liquid form such as tablets, capsules, powders, solutions, suspensions, emulsions and the like. Pharmaceutical compositions of the present invention may be administered orally, parenterally, subcutaneously, intravenously, intramuscularly, intraperitoneally, by nasal instillation, by implantation, by intracavitary or intravesical instillation, intraocularly, intraarterially, intralesionally, transdermally, or by the application to mucous membranes. In some embodiments, the composition may be applied to the nose, throat or bronchial tubes, for example by inhalation.

Optionally, the methods of the invention provide for the administration of a composition of the invention to a suitable animal model to identify the dosage of the composition(s), concentration of components therein and timing of administering the composition(s), which elicit tissue repair, reduce cell death, or induce another desirable biological response. Such determinations do not require undue experimentation, but are routine and can be ascertained without undue experimentation.

The biologically active agents can be conveniently provided to a subject as sterile liquid preparations, e.g., isotonic aqueous solutions, suspensions, emulsions, dispersions, or viscous compositions, which may be buffered to a selected pH. Cells and agents of the invention may be provided as liquid or viscous formulations. For some applications, liquid formations are desirable because they are convenient to administer, especially by injection. Where prolonged contact with a tissue is desired, a viscous composition may be preferred. Such compositions are formulated within the appropriate viscosity range. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions are prepared by suspending talampanel and/or perampanel in the required amount of the appropriate solvent with various amounts of the other ingredients, as desired. Such compositions may be in admixture with a suitable carrier, diluent, or excipient, such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON’S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells or agents present in their conditioned media.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent, such as methylcellulose. Other suitable thickening agents include, for example, xanthan gum, carboxymethyl cellulose, hydroxypropyl cellulose, carbomer, and the like. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form). Those skilled in the art will recognize that the components of the compositions should be selected to be chemically inert.

It should be understood that the method and compositions that would be useful in the present invention are not limited to the particular formulations set forth in the examples. The following examples are put forth so as to provide those of ordinary skill in the art with a complete disclosure and description of how to make and use the vectors and therapeutic methods of the invention, and are not intended to limit the scope of what the inventor(s) regard as their invention.

### Methods of Treatment

FSHD is a genetic muscle disorder involving the progressive degeneration of skeletal muscle that is inherited in an autosomal dominant manner. As implied by the name, FSHD primarily affects muscles of the face, shoulder blades, and upper arms, though it can affect other muscle groups. FSHD is the third most common type of muscular dystrophy, behind Duchenne and Becker and myotonic dystrophies. The incidence of FSHD is estimated to be approximately 4 per 100,000 births. The etiology of the disorder results from the loss of epigenetic control of the D4Z4 macrosatellite repeat array at chromosome 4q35 leading to the aberrant expression of the DUX4 gene in skeletal muscle cells. DUX4 is a transcription factor normally expressed only during embryonic development and epigenetically silenced as a consequence of the large number of repeats in the D4Z4 array. While DUX4 resides in every D4Z4 repeat unit, the full-length mRNA (DUX4-fl) can be stably expressed only by the distalmost repeat due to the presence of a functional polyadenylation signal.

Clinically, FSHD presents as muscle weakness and gradual atrophy, primarily affecting muscles of the face, shoulder, and upper arms, however muscles of the pelvis, hips, and lower leg can also be affected. Symptoms of FSHD can occur soon after birth, known as the infantile form, but often do not appear until puberty or young adulthood between ages 10-26. Rarely, symptoms can arise much later in life or, in some cases, not at all. Signs and symptoms of FSHD most often start as muscle weakness in the face and include eyelid drooping, inability to whistle due to weakness of the cheek muscles, decreased facial expression accompanied by difficulty pronouncing words. Severity of the symptoms often progress to the arms, shoulder blades, and legs resulting in the inability to reach above shoulder level, scapular winging, and sloping shoulders. Chronic pain is associated with advanced stages of the disorder and is present in 50% to 80% of cases. Hearing loss and heart arrhythmias may occur, but are not common. In extreme cases, FSHD results in patients being confined to a wheelchair and/or requiring ventilator support.

Relatively few treatments for FHSD are currently available and none are specific for the causes of the disease. While no current therapy can halt or reverse the effects of FHSD, therapeutic strategies can alleviate many of the symptoms of the disorder. Advanced cases of upper arm weakness and scapular winging can be stabilized by surgically fixing the scapulae to the rib cage. While this procedure limits arm movement, it can improve function by providing a solid leverage point for arm muscles. Weakening muscles in the upper and lower back can be stabilized and compensated by the use of a number of orthotic devices in the form of back supports, corsets, and girdles. Similarly, lower leg braces and ankle-foot orthotic devices can help preserve balance and mobility.

Mechanistically, FSHD can be broadly classified into two forms. FSHD1, the most common form of the disease, is caused by genetic shortening of the D4Z4 macrosatellite array, resulting in relaxation of chromatin that is normally repressed. FSHD2 is caused by mutations in proteins that maintain epigenetic silencing. In both cases, the resulting expression of DUX4-fl protein activates a host of genes normally expressed in early development, which causes the pathology when ectopically expressed in adult skeletal muscle.

Some aspects of the current invention relate to methods of treating FSHD in a subject in need thereof. In some embodiments, the method comprises administering to the subject an effective amount of a repressor of DUX4 gene expression, wherein the repressor decreases DUX4 gene expression in the skeletal muscle cells of the subject. In some embodiments, the DUX4 repressor is in the form of a CRISPRi platform comprising a sgRNA and a fusion protein further comprising a dCas9 protein fused to an epigenetic repressor. In some embodiments, the sgRNA directs the epigenetic repressor to the D4Z4 locus. In some embodiments, the localization of the repressor to the D4Z4 locus leads to epigenetic modifications to the chromatin of the locus, resulting in repression of DUX4 expression, thereby reducing or reversing the severity of the FSHD condition.

In some embodiments, the epigenetic repressor is a chromatin modifier that chemically alters the structure of the DNA backbone or post-translationally modifies histone proteins. Examples of epigenetic chromatin modifiers include, but are not limited to histone demethylases, histone methyltransferases, histone deacetylases, histone acetyltransferases, certain bromodomain-containing proteins, kinases that act to phosphorylate histones, and actin-dependent chromatin regulators. In some embodiments the chemical alteration of the DNA includes methylation of the C5 position of cytosine residues in CpG dinucleotide sequences. In some embodiments, the resulting modification of locus chromatin increases the number and density of epigenetic marks or tags associated with the DNA, which in turn induce a more “closed” or “tight” structure that inhibits transcription of the genes of the locus. In some embodiments, the binding of the dCas9 fusion protein to the D4Z4 locus further results in decreased gene expression through the physical blockade of enhancer and promoter proteins access to their DNA binding sites. These mechanisms of inhibition serve to at least partially restore the epigenetic silencing of the D4Z4 locus. In some embodiments, examples of epigenetic repressors that are used in the invention include, but are not limited to HP1 family proteins, including the chromo shadow domain and C-terminal extension regions of HP1α and HP1γ. In some embodiments, the epigenetic repressor comprises the transcription repression domain (TRD) of the methyl-CpG-binding protein MeCP2. In some embodiments, the epigenetic repressor comprises the SET domain of the histone-lysine N-methyltransferase protein SUV39H1. In some embodiments, the epigenetic repressor further comprises the pre- and post-SET domains of SUV39H1 in addition to the enzymatically active SET domain.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods are now described.

Antibodies. The ChIP-grade antibodies used in this study, α-KAP1 (ab3831), α-HP1α (ab77256), α-RNA Pol II CTD repeat (phospho S2) (ab5095), and α-histone H3 (ab1791) were purchased from Abcam (Cambridge, MA).

Plasmids. dSaCas9 constructs were designed with a muscle-specific regulatory cassette consisting of three modified CKM enhancers in tandem, upstream of a modified CKM promoter (Himeda, et al. (2021) Mol Ther, In press). Enhancer modifications are as follows: 1) Left E-box mutated to Right E-box (Nguyen, et al. (2003) J Biol Chem. 278: 46494-505); 2) enhancer CArG & AP2 sites removed; 3) 63 bp between Right E-box & MEF2 site removed (Salva, et al. (2007) Mol Ther. 15: 320-9); 4) sequence between TF binding motifs minimized; 5) +1 to +50 promoter sequence used (Salva, et al. (2007) Mol Ther. 15: 320-9); and 6) consensus Inr site added. This regulatory cassette was designed upstream of the SV40 bipartite nuclear localization signal flanking dSaCas9, which was fused in-frame to one of four epigenetic repressors (the SUV39H1 pre-SET, SET, and post-SET domains, the MeCP2 TRD, HP1α, or HP1γ) and HA tag, followed by the SV40 late pA signal. sgRNA constructs were designed with the U6 promoter followed by sgRNA, an SaCas9-optimized scaffold, and cPPT/CTS. All-in-one plasmid constructs contained the muscle regulatory cassette, the dSa-Cas9 fusion, and the U6-sgRNA on the same plasmid. Constructs were synthesized by GenScript in pUC57 and cloned into a pRRLSIN lentiviral (LV) vector for infection of primary FSHD myocytes or into pAAV-CA for AAV infection of mice. pRRLSIN.cPPT.PGK-GFP.WPRE was a gift from Didier Trono (Addgene plasmid # 12252 ; http://n2t.net/addgene:12252 ; RRID:Addgene_12252). pAAV-CA was a gift from Naoshige Uchida (Addgene plasmid # 69616; https://www.addgene.org/69616/ ; RRID: Addgene_69616).

sgRNA design and plasmid construction. The publically available sgRNA design tool from the Broad Institute (https://portals.broadinstitute.org/gpp/public/analysis-tools/sgma-design) was used to design dSaCas9-compatible sgRNAs targeting across the DUX4 locus. sgRNAs were cloned individually into BfuAI sites in the parent construct and sequence-verified or synthesized directly into the all-in-one plasmid and sequence-verified. The publically available Cas-OFFinder tool (http://www.rgenome.net/cas-offinder/) was then used to search for the closest-matching OT sequences in genes expressed in skeletal muscle. Refer to Table 1 for additional details.

Cell culture, transient transfections, LV infections, and AAV infections. Myogenic cells derived from biceps muscle of an FSHD1 patient (17Abic) were obtained from the Wellstone FSHD cell repository housed at the University of Massachusetts Medical School and grown as described (Himeda, et al. (2016)Mol Ther. 24: 527-35). 293T packaging cells were grown and transfected as described (Himeda, et al. (2016) Mol Ther. 24: 527-35). At ~70-80% confluency, 17Abic myoblasts were subjected to four serial infections as described (Himeda, et al. (2016) Mol Ther. 24: 527-35). Cells were harvested ~72 h following the last round of infection. pAAV-CA plasmids were used to generate infectious AAV9 viral particles (Vector Biolabs).

Quantitative reverse transcriptase PCR (qRT-PCR). Total RNAs were extracted using TRIzol (Invitrogen) and purified using the RNeasy Mini kit (Qiagen) after on-column DNase I digestion. Total RNA (2 µg) was used for cDNA synthesis using Superscript III Reverse Transcriptase (Invitrogen), and 200 ng of cDNA were used for qPCR analysis as described (Jones, et al. (2015) Clin Epigenetics. 7: 37). Oligonucleotide primer sequences are provided in Table 2.

Chromatin immunoprecipitation (ChIP). ChIP assays were performed with LV-infected 17Abic differentiated myocytes using the Fast ChIP method as described (Himeda, et al. (2016) Mol Ther. 24: 527-35). Chromatin was immunoprecipitated using 2 µg of specific antibodies. SYBR green quantitative PCR assays were performed as described (Himeda, et al. (2016) Mol Ther. 24: 527-35). Oligonucleotide primer sequences are provided in Table 2.

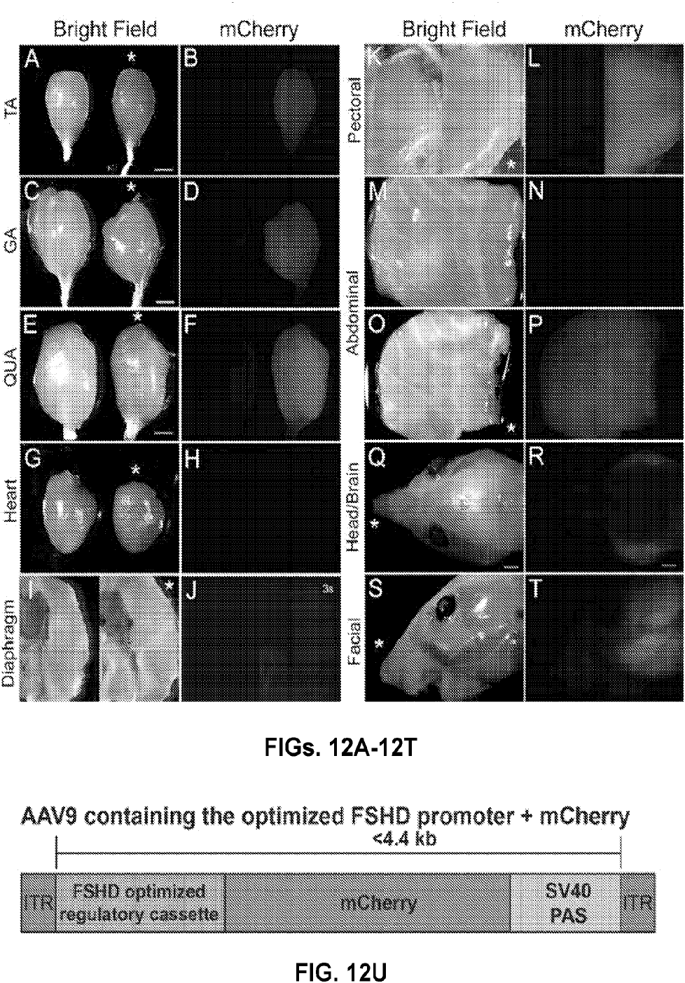

AAV injection and visualization. The FSHD optimized gene expression cassette regulating mCherry (FIG. 12U) was cloned between the AAV2 ITRs (using MluI and RsrII) of the pAAV-CA plasmid, a gift from Naoshige Uchida (Addgene plasmid # 69616 ; http://n2t.net/addgene:69616 ; RRID:Addgene_69616) and the construct was sent to Vector Biolabs (Malvern, PA) for AAV9 production. The AAV9-FSHD-mCherry vector (100 µl of 3.2 × 10¹³ GC/ml) was injected into the ophthalmic venous sinus of wild-type C57BL/6J mice at 3.5 weeks of age. The average AAV dose per body weight was 2.8 × 10¹¹ GC/kg. At 12 weeks post AAV injection, blood was removed by transcardial perfusion of PBS, and tissues were sampled for imaging and molecular analysis. The mCherry signals in all tissues were captured with Leica MZ9.5 / DFC-7000T fluorescent imaging system and Leica LAS X software using the same exposure unless indicated. Images were assembled with Adobe Photoshop CS6, and exposures were adjusted equally. In addition, genomic DNA was isolated from individual tissues and viral genomes were quantified by qPCR (50 ng genomic DNA) using bGH primers and normalized to the endogenous single copy Rosa26 gene. Oligonucleotide primer sequences are provided in Table 2.

RNA-seq. FSHD myocytes (17Abic) were subjected to four serial co-infections with LV supernatants expressing dSaCas9 fused to either: 1) the SUV39H1 pre-SET, SET, and post-SET domains (SET), 2) the MeCP2 TRD, 3) HP1γ, 4) HP1α, or 5) the KRAB TRD, each in combination with LV expressing an sgRNA targeting DUX4. Cells were harvested ~72 h following the last round of infection. For all treatments, five separate experiments were performed, and reduction of DUX4-fl and DUX4-FL targets was confirmed by qRT-PCR prior to submitting samples for sequencing. RNA-seq analysis was performed by GeneWiz, LLC using the Illumina HiSeq 2 × 100 bp platform, which is ideal for identifying gene expression levels, splice variant expression, and the de novo transcriptome assembly (including un-annotated sequences). The rRNA depletion, library construction, sequencing, and initial analysis (mapping all sequence reads to the human genome, reading hit count measurements, and differential gene expression comparisons) were performed by GeneWiz. Sequence reads were trimmed to remove possible adapter sequences and nucleotides with poor quality using Trimmomatic v.0.36. The trimmed reads were mapped to the Homo sapiens GRCh38 reference genome available on ENSEMBL using the STAR aligner v.2.5.2b. The STAR aligner is a splice aligner that detects splice junctions and incorporates them to help align the entire read sequences. Unique gene hit counts were calculated by using featureCounts from the Subread package v. 1.5.2. Only unique reads that fell within exon regions were counted. Since a strand-specific library preparation was performed, the reads were strand-specifically counted. Comparisons of gene expression between groups of samples were performed using DESeq2, described below. A gene ontology analysis was performed on the statistically significant set of genes by implementing the software GeneSCF v.1.1-p2. The goa_human GO list was used to cluster the set of genes based on their biological processes and determine their statistical significance. To estimate the expression levels of alternatively spliced transcripts, the splice variant hit counts were extracted from the RNA-seq reads mapped to the genome. Differentially spliced genes were identified for groups with more than one sample by testing for significant differences in read counts on exons (and junctions) of the genes using DEXSeq. Volcano plots of differentially expressed genes were generated using Prism 7 (Graphpad).

AAV transduction of dSaCas9-TRD or -KRAB in ACTA1-MCM; FLExD bi-transgenic mice. Tibialis anterior (TA) muscles of 4 week-old male ACTA1-MCM; FLExD bi-transgenic animals were injected with various ratios of AAV9-dSaCas9-TRD or -KRAB and AAV9-sgRNAs. AAV-dSaCas9-TRD or -KRAB was injected at 5×10⁵ GC/TA for all experiments. At 3.5 weeks post-AAV injection, mice were subjected to intraperitoneal injections of 5 mg/kg of tamoxifen (TMX) to induce DUX4-fl expression in skeletal muscles. TA muscles were sampled at 14 days post-TMX injection for gene expression analysis.

Statistical Analysis. Experiments in primary cells were performed using at least four biological replicates (for qRT-PCR analysis) and at least three biological replicates (for ChIP analysis), and data were analyzed using an unpaired, two-tailed Student’s t-test (p values: *p<0.05, **p<0.01, ***p<0.001). RNA-seq analysis was performed by GeneWiz using five biological replicates, and comparisons of gene expression between groups of samples were performed using DESeq2. The Wald test was used to generate p-values and log2 fold changes. Genes with a p-value <0.05 and absolute log2 fold change >1 were called as DEGs for each comparison. Enrichment of GO terms was tested using Fisher exact test (GeneSCF v1.1-p2). Significantly enriched GO terms had an adjusted P-value <0.05 in the differentially expressed gene sets. For AAV transductions in mice, gene expression was analyzed using an unpaired, two-tailed Student’s t-test.

TABLE 1

Specificity of SaCas9-compatible sgRNAs targeting the FSHD locus

| SEQ ID NO: | sgRNA | Target | 21-nt sequence + PAM (NNGRRT) | Mismatches | Wobbles |
|---|---|---|---|---|---|
| 5. | 1 | DUX4 prom | CGGCCCCAGGCCTCGACGCCCTGGGGT | | |
| 6. | OT | LAAT1* | aGGCCCCAGG-CTCGcCGCCCCAGGAT | 2 | 1 |
| 7. | 5 | DUX4 exon 1 | CTGTGCAGCGCGGCCCCCGGCGGGGGT | | |
| 8. | OT | RRS1** | CTGT--AGCtCGGCCtCCGGCGTGGGT | 2 | 2 |
| 9. | OT | GNAI1** | C-TGCgGCGCGGCCaCCGGCGGGAGT | 2 | 2 |

Two dSaCas9-compatible sgRNAs used in this study had potential off-target (OT) matches (http://www.rgenome.net/cas-offinder/) in or near genes expressed in skeletal muscle, as indicated. *Intron 1 of Lysosomal amino acid transporter 1 homolog (LAAT1) contains a potential OT match to sgRNA #1. **The single exon of Ribosome biogenesis regulatory protein homolog (RRS1) and the downstream flanking sequence of Guanine nucleotide-binding protein G(i) subunit alpha-1 isoform 1 (GNAI1) contain potential OT matches to sgRNA #5.

TABLE 2

Oligonucleotide primers to human genes (5′ ➔ 3′)

| SEQ ID NO: | Type | Name | Sequence |
|---|---|---|---|
| 10. | qRT-PCR | DUX4-fl-F | GCTCTGCTGGAGGAGCTTTAGGA |
| 11. | qRT-PCR | DUX4-fl-R | CGCACTGCTCGCAGGTCTGCWGGT |
| 12. | qRT-PCR | DUX4-fl-nested-F | AGCTTTAGGACGCGGGGTTGGGAC |
| 13. | qRT-PCR | DUX4-fl-nested-R | GCAGGTCTGCWGGTACCTGG |
| 14. | qRT-PCR | TRIM43-F | ACCCATCACTGGACTGGTGT |
| 15. | qRT-PCR | TRIM43-R: | CACATCCTCAAAGAGCCTGA |
| 16. | qRT-PCR | MBD3L2-F: | GCGTTCACCTCTTTTCCAAG |
| 17. | qRT-PCR | MBD3L2-R: | GCCATGTGGATTTCTCGTTT |
| 18. | qRT-PCR | MYH1-F: | ACAGAAGCGCAATGTTGAAG |
| 19. | qRT-PCR | MYH1-R: | CACCTTTGCTTGCAGTTTGT |
| 20. | qRT-PCR | FRG1-F: | TCTACAGAGACGTAGGCTGTCA |
| 21. | qRT-PCR | FRG1-R: | CTTGAGCACGAGCTTGGTAG |
| 22. | qRT-PCR | FRG2-F: | GGGAAAACTGCAGGAAAA |
| 23. | qRT-PCR | FRG2-R: | CTGGACAGTTCCCTGCTGTGT |
| 24. | qRT-PCR | LAAT1-F: | TCTGCTTTGCTGCATCTACC |
| 25. | qRT-PCR | LAAT1-R: | AGTACAGCGTCAGCATCACC |
| 26. | qRT-PCR | RRS1-F: | CACAACCGAGACTTTGGAGA |
| 27. | qRT-PCR | RRS1-R: | TCCCGCTCTGATACACAAAC |
| 28. | qRT-PCR | GNAI1-F: | CATCCCGACTCAACAAGATG |
| 29. | qRT-PCR | GNAI1-R: | TGCATTCGGTTCATTTCTTC |
| 30. | ChIP | DUX4 prom-F: | CCTGTTGCTCACGTCTCTCC |
| 31. | ChIP | DUX4 prom-R: | GTGGGGAGTCTGCAGTGTG |
| 32. | ChIP | DUX4 TSS-F: | GACACCCTCGGACAGCAC |
| 33. | ChIP | DUX4 TSS-R: | GTACGGGTTCCGCTCAAAG |
| 34. | ChIP | DUX4 exon3-F | CTGACGTGCAAGGGAGCT |
| 35. | ChIP | DUX4 exon3-R | CAGGTTTGCCTAGACAGCG |
| 36. | ChIP | 4-spec D4Z4-F | TCTGCTGGAGGAGCTTTAG |
| 37. | ChIP | 4-spec D4Z4-R | GAATGGCAGTTCTCCGCG* |
| 38. | CRISPRi** | sgRNA-1 | CGGCCCCAGGCCTCGACGCCC |
| 39. | CRISPRi** | sgRNA-2 | TCGACGCCCTGGGGTCCCTTC |
| 40. | CRISPRi** | sgRNA-3 | TCCGCGGGGAGGGTGCTGTCC |
| 41. | CRISPRi** | sgRNA-4 | GCCAGCTGAGGCAGCACCGGC |
| 42. | CRISPRi** | sgRNA-5 | CTGTGCAGCGCGGCCCCCGGC |
| 43. | CRISPRi** | sgRNA-6 | TCATCCAGCAGCAGGCCGCAG |
| 44. | AAV qPCR | bGH-F | TCTAGTTGCCAGCCATCTGTTGT |
| 45. | AAV qPCR | bGH-R | TGGGAGTGGCACCTTCCA |
| 46. | AAV qPCR | Rosa26-F | CAATACCTTTCTGGGAGTTCTCTGCTGC |
| 47. | NAV qPCR | Rosa26-R | TGCAGGACAACGCCCACACACC |

*G at this position is specific to chromosome 4 (G) vs chromosome 10 (T)
**Each sgRNA is a 21-bp sequence preceded by a G for most effective targeting

TABLE 3

dSaCas9-fusion proteins

| SEQ ID NO: | Name: | Type: | Amino Acid Sequence: |
|---|---|---|---|
| 1. | SUV39H1 SET | dSaCas9 Fusion Protein | MAPKKKRKVEASKRNYILGLAIGITSVGYGIIDYETRDV IDAGVRLFKEANVENNEGRRSKRGARRLKRRRRHRIQR VKKLLFDYNLLTDHSELSGINPYEARVKGLSQKLSEEEF SAALLHLAKRRGVHNVNEVEEDTGNELSTKEQISRNSK ALEEKYVAELQLERLKKDGEVRGSINRFKTSDYVKEA KQLLKVQKAYHQLDQSFIDTYIDLLETRRTYYEGPGEG SPFGWKDIKEWYEMLMGHCTYFPEELRSVKYAYNADL YNALNDLNNLVITRDENEKLEYYEKFQIIENVFKQKKK PTLKQIAKEILVNEEDIKGYRVTSTGKPEFTNLKVYHDI KDITARKEIIENAELLDQIAKILTIYQSSEDIQEELTNLNS ELTQEEIEQISNLKGYTGTHNLSLKAINLILDELWHTND NQIAIFNRLKLVPKKVDLSQQKEIPTTLVDDFILSPVVK RSFIQSIKVINAIIKKYGLPNDIIIELAREKNSKDAQKMIN EMQKRNRQTNERIEEIIRTTGKENAKYLIEKIKLHDMQE GKCLYSLEAIPLEDLLNNPFNYEVDHIIPRSVSFDNSFNN KVLVKQEEASKKGNRTPFQ LSSSDSKISYETFKKHILN LAKGKGRISKTKKEYLLEERDINRFSVQKDFINRNLVDT RYATRGLMNLLRSYFRVNNLDVKVKSINGGFTSFLRRK WKFKKERNKGYKHHAEDALIIANADFIFKEWKKLDKA KKVMENQMFEEKQAESMPEIETEQEYKEIFITPHQIKHI KDFKDYKYSHRVDKKPNRELINDTLYSTRKDDKGNTLI VNNLNGLYDKDNDKLKKLINKSPEKLLMYHHDPQTYQ KLKLIMEQYGDEKNPLYKYYEETGNYLTKYSKKDNGP VIKKIKYYGNKLNAHLDITDDYPNSRNKVVKLSLKPYR FDVYLDNGVYKFVTVKNLDVIKKENYYEVNSKCYEEA KKLKKISNQAEFIASFYNNDLIKINGELYRVIGVNNDLL NRIEVNMIDITYREYLENMNDKRPPRIIKTIASKTQ SIKK YSTDILGNLYEVKSKKHPQIIKKGGTGGPKKKRKVGRA YDLCIFRTDDGRGWGVRTLEKIRKNSFVMEYVGEIITSE EAERRGQIYDRQGATYLFDLDYVEDVYTVDAAYYGNI SHFVNHSCDPNLQVYNVFIDNLDERLPRYPYDVPDYA |
| 2. | MeCP2 TRD | dSaCas9 Fusion Protein | MAPKKKRKVEASKRNYILGLAIGITSVGYGIIDYETRDV IDAGVRLFKEANVENNEGRRSKRGARRLKRRRRHRIQR VKKLLFDYNLLTDHSELSGINPYEARVKGLSQKLSEEEF SAALLHLAKRRGVHNVNEVEEDTGNELSTKEQISRNSK ALEEKYVAELQLERLKKDGEVRGSINRFKTSDYVKEA KQLLKVQKAYHQLDQSFIDTYIDLLETRRTYYEGPGEG SPFGWKDIKEWYEMLMGHCTYFPEELRSVKYAYNADL YNALNDLNNLVITRDENEKLEYYEKFQIIENVFKQKKK PTLKQIAKEILVNEEDIKGYRVTSTGKPEFTNLKVYHDI |
| | | | KDITARKEIIENAELLDQIAKILTIYQSSEDIQEELTNLNS ELTQEEIEQISNLKGYTGTHNLSLKAINLILDELWHTND NQIAIFNRLKLVPKKVDLSQQKEIPTTLVDDFILSPVVK RSFIQSIKVINAIIKKYGLPNDIIIELAREKNSKDAQKMIN EMQKRNRQTNERIEEIIRTTGKENAKYLIEKIKLHDMQE GKCLYSLEAIPLEDLLNNPFNYEVDHIIPRSVSFDNSFNN KVLVKQEEASKKGNRTPFQ LSSSDSKISYETFKKHILN LAKGKGRISKTKKEYLLEERDINRFSVQKDFINRNLVDT RYATRGLMNLLRSYFRVNNLDVKVKSINGGFTSFLRRK WKFKKERNKGYKHHAEDALIIANADFIFKEWKKLDKA KKVMENQMFEEKQAESMPEIETEQEYKEIFITPHQIKHI KDFKDYKYSHRVDKKPNRELINDTLYSTRKDDKGNTLI VNNLNGLYDKDNDKLKKLINKSPEKLLMYHHDPQTYQ KLKLIMEQYGDEKNPLYKYYEETGNYLTKYSKKDNGP VIKKIKYYGNKLNAHLDITDDYPNSRNKVVKLSLKPYR FDVYLDNGVYKFVTVKNLDVIKKENYYEVNSKCYEEA KKLKKISNQAEFIASFYNNDLIKINGELYRVIGVNNDLL NRIEVNMIDITYREYLENMNDKRPPRIIKTIASKTQ SIKK YSTDILGNLYEVKSKKHPQIIKKGGTGGPKKKRKVGRA GRKPGSVVAAAAAEAKKKAVKESSIRSVQETVLPIKKR KTRYPYDVPDYA |
| 3. | HP1α chromo shadow and CTE | dSaCas9 Fusion Protein | MAPKKKRKVEASKRNYILGLAIGITSVGYGIIDYETRDV IDAGVRLFKEANVENNEGRRSKRGARRLKRRRRHRIQR VKKLLFDYNLLTDHSELSGINPYEARVKGLSQKLSEEEF SAALLHLAKRRGVHNVNEVEEDTGNELSTKEQISRNSK ALEEKYVAELQLERLKKDGEVRGSINRFKTSDYVKEA KQLLKVQKAYHQLDQSFIDTYIDLLETRRTYYEGPGEG SPFGWKDIKEWYEMLMGHCTYFPEELRSVKYAYNADL YNALNDLNNLVITRDENEKLEYYEKFQIIENVFKQKKK PTLKQIAKEILVNEEDIKGYRVTSTGKPEFTNLKVYHDI KDITARKEIIENAELLDQIAKILTIYQSSEDIQEELTNLNS ELTQEEIEQISNLKGYTGTHNLSLKAINLILDELWHTND NQIAIFNRLKLVPKKVDLSQQKEIPTTLVDDFILSPVVK RSFIQSIKVINAIIKKYGLPNDIIIELAREKNSKDAQKMIN EMQKRNRQTNERIEEIIRTTGKENAKYLIEKIKLHDMQE GKCLYSLEAIPLEDLLNNPFNYEVDHIIPRSVSFDNSFNN KVLVKQEEASKKGNRTPFQ LSSSDSKISYETFKKHILN LAKGKGRISKTKKEYLLEERDINRFSVQKDFINRNLVDT RYATRGLMNLLRSYFRVNNLDVKVKSINGGFTSFLRRK WKFKKERNKGYKHHAEDALIIANADFIFKEWKKLDKA KKVMENQMFEEKQAESMPEIETEQEYKEIFITPHQIKHI KDFKDYKYSHRVDKKPNRELINDTLYSTRKDDKGNTLI VNNLNGLYDKDNDKLKKLINKSPEKLLMYHHDPQTYQ KLKLIMEQYGDEKNPLYKYYEETGNYLTKYSKKDNGP VIKKIKYYGNKLNAHLDITDDYPNSRNKVVKLSLKPYR FDVYLDNGVYKFVTVKNLDVIKKENYYEVNSKCYEEAKKLKKISNQAEFIASFYNNDLIKINGELYRVIGVNNDLL NRIEVNMIDITYREYLENMNDKRPPRIIKTIASKTQ SIKK YSTDILGNLYEVKSKKHPQIIKKGGTGGPKKKRKVGRA LEPEKIIGATDSCGDLMFLMKWKDTDEADLVLAKEAN VKCPQIVIAFYEERLTWHAYPEDAENKEKETAKSYPYD VPDYA |
| 4. | HP1γ chromo shadow and CTE | dSaCas9 Fusion Protein | MAPKKKRKVEASKRNYILGLAIGITSVGYGIIDYETRDV IDAGVRLFKEANVENNEGRRSKRGARRLKRRRRHRIQR VKKLLFDYNLLTDHSELSGINPYEARVKGLSQKLSEEEF SAALLHLAKRRGVHNVNEVEEDTGNELSTKEQISRNSK ALEEKYVAELQLERLKKDGEVRGSINRFKTSDYVKEA KQLLKVQKAYHQLDQSFIDTYIDLLETRRTYYEGPGEG SPFGWKDIKEWYEMLMGHCTYFPEELRSVKYAYNADL YNALNDLNNLVITRDENEKLEYYEKFQIIENVFKQKKK PTLKQIAKEILVNEEDIKGYRVTSTGKPEFTNLKVYHDI KDITARKEIIENAELLDQIAKILTIYQSSEDIQEELTNLNS ELTQEEIEQISNLKGYTGTHNLSLKAINLILDELWHTND NQIAIFNRLKLVPKKVDLSQQKEIPTTLVDDFILSPVVK RSFIQSIKVINAIIKKYGLPNDIIIELAREKNSKDAQKMIN EMQKRNRQTNERIEEIIRTTGKENAKYLIEKIKLHDMQE GKCLYSLEAIPLEDLLNNPFNYEVDHIIPRSVSFDNSFNN KVLVKQEEASKKGNRTPFQ LSSSDSKISYETFKKHILN LAKGKGRISKTKKEYLLEERDINRFSVQKDFINRNLVDT RYATRGLMNLLRSYFRVNNLDVKVKSINGGFTSFLRRK WKFKKERNKGYKHHAEDALIIANADFIFKEWKKLDKA KKVMENQMFEEKQAESMPEIETEQEYKEIFITPHQIKHI KDFKDYKYSHRVDKKPNRELINDTLYSTRKDDKGNTLI VNNLNGLYDKDNDKLKKLINKSPEKLLMYHHDPQTYQ KLKLIMEQYGDEKNPLYKYYEETGNYLTKYSKKDNGP VIKKIKYYGNKLNAHLDITDDYPNSRNKVVKLSLKPYR FDVYLDNGVYKFVTVKNLDVIKKENYYEVNSKCYEEA KKLKKISNQAEFIASFYNNDLIKINGELYRVIGVNNDLL NRIEVNMIDITYREYLENMNDKRPPRIIKTIASKTQ SIKK YSTDILGNLYEVKSKKHPQIIKKGGTGGPKKKRKVGRA LDPERIIGATDSSGELMFLMKWKDSDEADLVLAKEAN MKCPQIVIAFYEERLTWHSCPEDEAQYPYDVPDYA |

TABLE 4

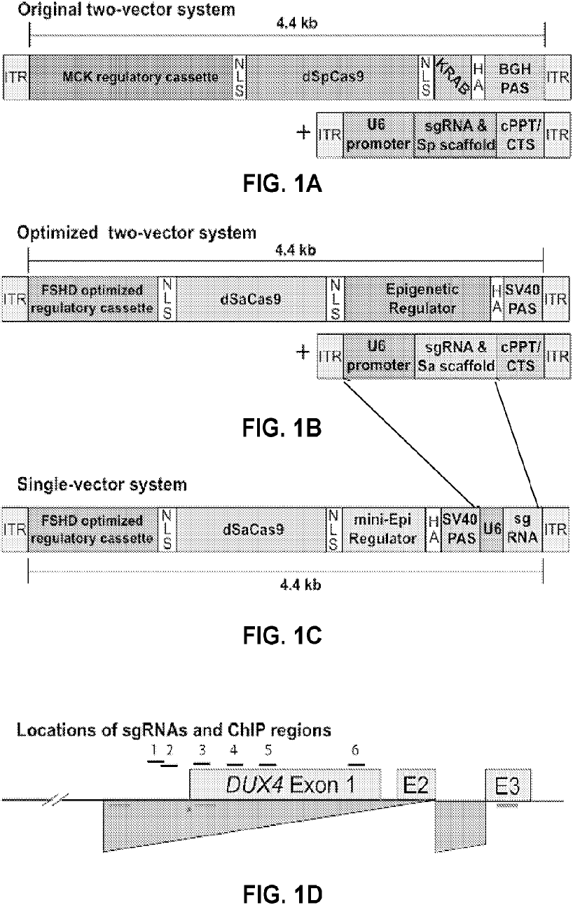

Gene expression regulatory cassettes (FIG. 1C seqs for the single vector system)

| SEQ ID NO: | Name: | Type: | DNA Sequence: |
|---|---|---|---|
| 48. | All-in-one CLH 17v2 (mouseCKM-TRD) | AAV Skeletal Muscle Vector | acgcgtgatatcggacacccgagatgcctggttataattaacccAGACATGTGG CTGccccccccaacacctgctgcgagctctaaaaataaccctgggacacccgagatg cctggttataattaacccAGACATGTGGCTGccccccccaacacctgctgcga gctctaaaaataaccctgggacacccgagatgcctggttataattaacccAGACATGTGGCTGccccccccaacacctgctgcgagctctaaaaataacccctccctgggg acagcccctcctggctagtcacaccctgtaggctcctctatataacccaggggcacaggg gctgcccTCATTCTACCACCACCTCCACAGCACAGACAGA CACTCAGGAGCCAGCCAGCGCCACCATGGCCCCCAAG AAGAAGAGGAAGGTGGAGGCCAGCAAGCGGAACTAC ATCCTGGGCCTGGCCATCGGCATCACCAGCGTGGGCT ACGGCATCATCGACTACGAGACcCGGGACGTGATCGA cGCCGGCGTGCGGCTGTTCAAgGAGGCCAACGTGGAgA ACAACGAGGGCAGGCGGAGCAAGAGAGGCGCCAGAA GGCTGAAGCGGCGGAGGCGGCAcAGAATCCAGAGAGT GAAGAAGCTGCTGTTCGACTACAACCTGCTGACCGAC CACAGCGAGCTGAGCGGCATCAACCCCTACGAGGCCA GAGTGAAGGGCCTGAGCCAGAAGCTGAGCGAGGAgG AGTTCagcGCCGCCCTGCTGCACCTGGCCAAGAGAAGA GGCGTGCACAACGTGAACGAGGTGGAgGAGGACACCG GCAACGAGCTGTCCACCAAgGAGCAGATCAGCCGGAA CAGCAAGGCCCTGGAgGAGAAgTACGTGGCCGAgCTG CAGCTGGAgCGGCTGAAGAAgGACGGCGAgGTGCGGG GCAGCATCAACAGATTCAAGACCAGCGACTACGTGAA gGAgGCCAAgCAGCTGCTGAAGGTGCAGAAGGCCTAC CACCAGCTGGACCAGAGCTTCATCGACACCTACATCG ACCTGCTGGAgACCCGGCGGACCTACTAcGAGGGcCCc GGCGAGGGCAGCCCCTTCGGCTGGAAGGACATCAAgG AgTGGTACGAGATGCTGATGGGCCACTGCACCTACTTC CCCGAGGAgCTGCGGAGCGTGAAGTACGCCTACAACG CCGACCTGTACAACGCCCTGAACGACCTGAACAAcCTg GTGATCACCAGGGACGAGAACGAGAAGCTGGAgTAcT ACGAGAAGTTCCAGATCATCGAGAACGTGTTCAAGCA GAAGAAGAAGCCCACCCTGAAGCAGATCGCCAAgGAg ATCCTgGTGAACGAgGAGGAcATcAAGGGCTACAGAGT GACCAGCACCGGCAAGCCCGAGTTCACCAACCTGAAG GTGTACCACGACATCAAGGACATcACCGCCCGGAAgG AGATcATcGAGAACGCCGAGCTGCTGGAcCAGATcGCC AAGATCCTGACCATCTACCAGAGCAGCGAGGACATCC AGGAgGAgCTGACCAAcCTGAACTCCGAGCTGACCCAG GAgGAGATCGAGCAGATCagcAAcCTGAAGGGCTAcAC CGGCACCCACAACCTGAGCCTGAAGGCCATCAACCTG ATCCTGGACGAGCTGTGGCACACCAACGACAACCAGA TCGCcATCTTCAACCGGCTGAAGCTGGTGCCCAAGAA GGTGGACCTGTCCCAGCAGAAgGAGATCCCCACCACC CTGGTGGACGACTTCATCCTGAGCCCCGTgGTGAAGA GAAGCTTCATCCAGAGCATCAAgGTGATCAACGCCAT CATCAAGAAGTACGGCCTGCCCAACGACATCATcATC GAGCTGGCCCGgGAGAAGAACTCCAAGGACGCCCAGA AgATGATCAACGAGATGCAGAAGCGGAACCGGCAGAC CAACGAGCGGATCGAGGAgATCATCCGGACCACCGGCAAgGAGAACGCCAAGTACCTGATCGAGAAGATCAAGC TGCACGACATGCAGGAgGGCAAGTGCCTGTACAGCCT GGAgGCCATCCCcCTGGAgGAcCTGCTGAACAACCCCT TCAACTAcGAGGTGGACCACATCATCCCCAGAAGCGT GTCCTTCGACAACAGCTTCAACAACAAGGTGCTgGTG AAGCAGGAgGAgGCCAGCAAGAAGGGCAACCGGACCC CcTTCCAGTACCTGAGCAGCAGCGACAGCAAGATCAG CTACGAgACCTTCAAGAAGCACATCCTGAAcCTGGCCA AGGGCAAGGGCAGAATCAGCAAGACCAAGAAgGAGT AcCTGCTGGAgGAgCGGGACATCAACAGGTTCTCCGTG CAGAAgGACTTCATCAACCGGAACCTGGTGGAcACCA GATACGCCACCAGAGGCCTGATGAACCTGCTGCGGAG CTACTTCAGAGTGAACAACCTGGACGTGAAgGTGAAG TCCATCAAcGGCGGCTTCACCAGCTTcCTGCGGCGGAA GTGGAAGTTcAAGAAgGAGCGGAACAAGGGcTACAAG CACCACGCCGAGGACGCCCTGATCATcGCCAACGCCG AcTTCATCTTCAAgGAGTGGAAGAAgCTGGACAAGGCC AAgAAgGTGATGGAgAACCAGATGTTCGAGGAgAAGC AGGCCGAGAGCATGCCCGAGATCGAgACCGAGCAGGA GTACAAgGAGATCTTCATCACCCCCCACCAGATCAAG CACATcAAGGACTTCAAGGACTACAAGTACAGCCACC GGGTGGACAAGAAGCCcAAcAGAGAGCTGATcAACGA CACCCTGTACTCCACCCGGAAGGACGACAAGGGCAAC ACCCTGATCGTGAACAAcCTGAACGGCCTGTACGACA AGGACAAcGACAAGCTGAAgAAGCTGATCAACAAGAG CCCCGAgAAGCTGCTGATGTACCACCACGACCCCCAG ACCTACCAGAAgCTGAAGCTGATcATGGAgCAGTACGG CGACGAGAAGAAcCCCCTGTACAAGTACTACGAGGAg ACCGGcAACTACCTGACCAAGTACTCCAAgAAGGACA ACGGCCCCGTGATCAAGAAGATcAAGTAcTACGGCAA CAAgCTGAACGCCCAcCTGGACATCACCGACGACTACC CCAACAGCAGAAACAAGGTgGTGAAGCTGTCCCTGAA GCCCTACAGATTCGACGTGTACCTGGACAAcGGCGTGT ACAAGTTCGTGACCGTGAAGAAcCTGGAcGTGATCAAg AAgGAgAACTACTACGAgGTGAAcAGCAAGTGCTAcGA GGAgGCcAAGAAGCTGAAGAAGATCAGCAACCAGGCC GAGTTcATCGCCTCCTTCTACAACAACGAcCTGATCAA GATCAACGGCGAGCTGTAcAGAGTGATCGGCGTGAAC AACGACCTGCTGAACCGGATCGAgGTGAACATGATCG ACATCACCTACCGCGAGTACCTGGAgAACATGAACGA CAAGAGGCCCCCCAGGATCATcAAGACcATCGCCTCCA AGACCCAGAGCATcAAGAAGTACAGCACcGACATcCTG GGCAACCTGTAcGAgGTGAAgTCcAAGAAGCACCCcCA GATCATCAAgAAGGGCGGCACCGGCGGCCCCAAGAAG AAGAGGAAGGTGGGCCGGGCCGGCCGGAAGCCCGGC AGCGTGGTGGCCGCCGCCGCCGCCGAGGCCAAGAAGAAGGCCGTGAAGGAGAGCAGCATCCGGAGCGTGCAG GAGACCGTGCTGCCCATCAAGAAGCGGAAGACCAGAT ACCCCTACGACGTGCCCGACTACGCCTGATATTTGTGA AATTTGTGATGCTATTGCTTTATTTGTAACCATCTAGC TTTATTTGTGAAATTTGTGATGCTATTGCTTTATTTGTA ACCATTTTATTTGTGAAATTTGTGATGCTATTGCTTTAT TTGTAACCATTATAAGCTGCAATAAACAAGTTAACAA CAACAATTGCATTCATTTTATGTTTCAGGTTCAGGGGG AGATGTGGGAGGTTTTTTAAAGCGGGAGGGCCTATTT CCCATGATTCCTTCATATTTGCATATACGATACAAGGC TGTTAGAGAGATAATTAGAATTAATTTGACTGTAAAC ACAAAGATATTAGTACAAAATACGTGACGTAGAAAGT AATAATTTCTTGGGTAGTTTGCAGTTTTAAAATTATGT TTTAAAATGGACTATCATATGCTTACCGTAACTTGAAA GTATTTCGATTTCTTGGCTTTATATATCTTGTGGAAAG GACGaaacaccgNNNNNNNNNNNNNNNNNNNNNGTTTAA GTACTCTGTGCTGGAAACAGCACAGAATCTACTTAAA CAAGGCAAAATGCCGTGTTTATCTCGTCAACTTGTTGG CGAGATTTTTTTtaccacc |
| 49. | All-in-one CLH 17v2 (human CKM-TRD) | AAV Skeletal Muscle Vector | acgcgtgatatcggacacccgagaCgccCggttataattaaccAGGACACGT GGCGAccccccccaacacctgcCcgaCctctaaaaataacTcCTggacacccg agaCgccCggttataattaaccAGGACACGTGGCGAccccccccaacacc tgcCcgaCctctaaaaataacTcCTggacacccgagaCgccCggttataattaacc AGGACACGTGGCGAccccccccaacacctgcCcgaCctctaaaaataacc cctccctggggacaAcccctccCAgcCaAtAGcacAGCCtaggTCccCcta tataaGGccaCggCTGCTGGCCCTTCCTCATTCTCAGTGTCA CCTCCAGGATACAGACAGCCCCCCTTCAGCCCAGCCC GCCACCATGGCCCCCAAGAAGAAGAGGAAGGTGGAG GCCAGCAAGCGGAACTACATCCTGGGCCTGGCCATCG GCATCACCAGCGTGGGCTACGGCATCATCGACTACGA GACcCGGGACGTGATCGAcGCCGGCGTGCGGCTGTTCA AgGAGGCCAACGTGGAgAACAACGAGGGCAGGCGGA GCAAGAGAGGCGCCAGAAGGCTGAAGCGGCGGAGGC GGCAcAGAATCCAGAGAGTGAAGAAGCTGCTGTTCGA CTACAACCTGCTGACCGACCACAGCGAGCTGAGCGGC ATCAACCCCTACGAGGCCAGAGTGAAGGGCCTGAGCC AGAAGCTGAGCGAGGAgGAGTTCagcGCCGCCCTGCTG CACCTGGCCAAGAGAAGAGGCGTGCACAACGTGAAC GAGGTGGAgGAGGACACCGGCAACGAGCTGTCCACCA AgGAGCAGATCAGCCGGAACAGCAAGGCCCTGGAgGA GAAgTACGTGGCCGAgCTGCAGCTGGAgCGGCTGAAG AAgGACGGCGAgGTGCGGGGCAGCATCAACAGATTCA AGACCAGCGACTACGTGAAgGAgGCCAAgCAGCTGCT GAAGGTGCAGAAGGCCTACCACCAGCTGGACCAGAG CTTCATCGACACCTACATCGACCTGCTGGAgACCCGGC GGACCTACTAcGAGGGcCCcGGCGAGGGCAGCCCCTTCGGCTGGAAGGACATCAAgGAgTGGTACGAGATGCTGA TGGGCCACTGCACCTACTTCCCCGAGGAgCTGCGGAG CGTGAAGTACGCCTACAACGCCGACCTGTACAACGCC CTGAACGACCTGAACAAcCTgGTGATCACCAGGGACG AGAACGAGAAGCTGGAgTAcTACGAGAAGTTCCAGAT CATCGAGAACGTGTTCAAGCAGAAGAAGAAGCCCACC CTGAAGCAGATCGCCAAgGAgATCCTgGTGAACGAgGA GGAcATcAAGGGCTACAGAGTGACCAGCACCGGCAAG CCCGAGTTCACCAACCTGAAGGTGTACCACGACATCA AGGACATcACCGCCCGGAAgGAGATcATcGAGAACGCC GAGCTGCTGGAcCAGATcGCCAAGATCCTGACCATCTA CCAGAGCAGCGAGGACATCCAGGAgGAgCTGACCAAc CTGAACTCCGAGCTGACCCAGGAgGAGATCGAGCAGA TCagcAAcCTGAAGGGCTAcACCGGCACCCACAACCTGA GCCTGAAGGCCATCAACCTGATCCTGGACGAGCTGTG GCACACCAACGACAACCAGATCGCcATCTTCAACCGG CTGAAGCTGGTGCCCAAGAAGGTGGACCTGTCCCAGC AGAAgGAGATCCCCACCACCCTGGTGGACGACTTCAT CCTGAGCCCCGTgGTGAAGAGAAGCTTCATCCAGAGC ATCAAgGTGATCAACGCCATCATCAAGAAGTACGGCC TGCCCAACGACATCATcATCGAGCTGGCCCGgGAGAA GAACTCCAAGGACGCCCAGAAgATGATCAACGAGATG CAGAAGCGGAACCGGCAGACCAACGAGCGGATCGAG GAgATCATCCGGACCACCGGCAAgGAGAACGCCAAGT ACCTGATCGAGAAGATCAAGCTGCACGACATGCAGGA gGGCAAGTGCCTGTACAGCCTGGAgGCCATCCCcCTGG AgGAcCTGCTGAACAACCCCTTCAACTAcGAGGTGGAC CACATCATCCCCAGAAGCGTGTCCTTCGACAACAGCT TCAACAACAAGGTGCTgGTGAAGCAGGAgGAgGCCAG CAAGAAGGGCAACCGGACCCCcTTCCAGTACCTGAGC AGCAGCGACAGCAAGATCAGCTACGAgACCTTCAAGA AGCACATCCTGAAcCTGGCCAAGGGCAAGGGCAGAAT CAGCAAGACCAAGAAgGAGTAcCTGCTGGAgGAgCGG GACATCAACAGGTTCTCCGTGCAGAAgGACTTCATCA ACCGGAACCTGGTGGAcACCAGATACGCCACCAGAGG CCTGATGAACCTGCTGCGGAGCTACTTCAGAGTGAAC AACCTGGACGTGAAgGTGAAGTCCATCAAcGGCGGCTT CACCAGCTTcCTGCGGCGGAAGTGGAAGTTcAAGAAgG AGCGGAACAAGGGcTACAAGCACCACGCCGAGGACGC CCTGATCATcGCCAACGCCGAcTTCATCTTCAAgGAGT GGAAGAAgCTGGACAAGGCCAAgAAgGTGATGGAgAA CCAGATGTTCGAGGAgAAGCAGGCCGAGAGCATGCCC GAGATCGAgACCGAGCAGGAGTACAAgGAGATCTTCA TCACCCCCCACCAGATCAAGCACATcAAGGACTTCAA GGACTACAAGTACAGCCACCGGGTGGACAAGAAGCCc AAcAGAGAGCTGATcAACGACACCCTGTACTCCACCCGGAAGGACGACAAGGGCAACACCCTGATCGTGAACAAc CTGAACGGCCTGTACGACAAGGACAAcGACAAGCTGA AgAAGCTGATCAACAAGAGCCCCGAgAAGCTGCTGAT GTACCACCACGACCCCCAGACCTACCAGAAgCTGAAG CTGATcATGGAgCAGTACGGCGACGAGAAGAAcCCCCT GTACAAGTACTACGAGGAgACCGGcAACTACCTGACC AAGTACTCCAAgAAGGACAACGGCCCCGTGATCAAGA AGATcAAGTAcTACGGCAACAAgCTGAACGCCCAcCTG GACATCACCGACGACTACCCCAACAGCAGAAACAAG GTgGTGAAGCTGTCCCTGAAGCCCTACAGATTCGACGT GTACCTGGACAAcGGCGTGTACAAGTTCGTGACCGTG AAGAAcCTGGAcGTGATCAAgAAgGAgAACTACTACGA gGTGAAcAGCAAGTGCTAcGAGGAgGCcAAGAAGCTGA AGAAGATCAGCAACCAGGCCGAGTTcATCGCCTCCTTC TACAACAACGAcCTGATCAAGATCAACGGCGAGCTGT AcAGAGTGATCGGCGTGAACAACGACCTGCTGAACCG GATCGAgGTGAACATGATCGACATCACCTACCGCGAG TACCTGGAgAACATGAACGACAAGAGGCCCCCCAGGA TCATcAAGACcATCGCCTCCAAGACCCAGAGCATcAAG AAGTACAGCACcGACATcCTGGGCAACCTGTAcGAgGT GAAgTCcAAGAAGCACCCcCAGATCATCAAgAAGGGCG GCACCGGCGGCCCCAAGAAGAAGAGGAAGGTGGGCC GGGCCGGCCGGAAGCCCGGCAGCGTGGTGGCCGCCG CCGCCGCCGAGGCCAAGAAGAAGGCCGTGAAGGAGA GCAGCATCCGGAGCGTGCAGGAGACCGTGCTGCCCAT CAAGAAGCGGAAGACCAGATACCCCTACGACGTGCCC GACTACGCCTGATATTTGTGAAATTTGTGATGCTATTG CTTTATTTGTAACCATCTAGCTTTATTTGTGAAATTTGT GATGCTATTGCTTTATTTGTAACCATTTTATTTGTGAA ATTTGTGATGCTATTGCTTTATTTGTAACCATTATAAG CTGCAATAAACAAGTTAACAACAACAATTGCATTCAT TTTATGTTTCAGGTTCAGGGGGAGATGTGGGAGGTTTT TTAAAGCGGGAGGGCCTATTTCCCATGATTCCTTCATA TTTGCATATACGATACAAGGCTGTTAGAGAGATAATT AGAATTAATTTGACTGTAAACACAAAGATATTAGTAC AAAATACGTGACGTAGAAAGTAATAATTTCTTGGGTA GTTTGCAGTTTTAAAATTATGTTTTAAAATGGACTATC ATATGCTTACCGTAACTTGAAAGTATTTCGATTTCTTG GCTTTATATATCTTGTGGAAAGGACGaaacaccgNNNNNN NNNNNNNNNNNNNNNGTTTAAGTACTCTGTGCTGGAA ACAGCACAGAATCTACTTAAACAAGGCAAAATGCCGT GTTTATCTCGTCAACTTGTTGGCGAGATTTTTTTggtaccg gaccg |
| 50. | All-in-one CLH-22 (mouseCKM-HP1α) | AAV SkeletalMuscle Vector | acgcgtgatatcggacacccgagatgcctggttataattaacccAGACATGTGG CTGccccccccaacacctgctgcgagctctaaaaataaccctgggacacccgagatg cctggttataattaacccAGACATGTGGCTGccccccccaacacctgctgcgagctctaaaaataaccctgggacacccgagatgcctggttataattaacccAGACAT GTGGCTGccccccccaacacctgctgcgagctctaaaaataacccctccctgggg acagcccctcctggctagtcacaccctgtaggctcctctatataacccaggggcacaggg gctgcccTCATTCTACCACCACCTCCACAGCACAGACAGA CACTCAGGAGCCAGCCAGCGCCACCATGGCCCCCAAG AAGAAGAGGAAGGTGGAGGCCAGCAAGCGGAACTAC ATCCTGGGCCTGGCCATCGGCATCACCAGCGTGGGCT ACGGCATCATCGACTACGAGACcCGGGACGTGATCGA cGCCGGCGTGCGGCTGTTCAAgGAGGCCAACGTGGAgA ACAACGAGGGCAGGCGGAGCAAGAGAGGCGCCAGAA GGCTGAAGCGGCGGAGGCGGCAcAGAATCCAGAGAGT GAAGAAGCTGCTGTTCGACTACAACCTGCTGACCGAC CACAGCGAGCTGAGCGGCATCAACCCCTACGAGGCCA GAGTGAAGGGCCTGAGCCAGAAGCTGAGCGAGGAgG AGTTCagcGCCGCCCTGCTGCACCTGGCCAAGAGAAGA GGCGTGCACAACGTGAACGAGGTGGAgGAGGACACCG GCAACGAGCTGTCCACCAAgGAGCAGATCAGCCGGAA CAGCAAGGCCCTGGAgGAGAAgTACGTGGCCGAgCTG CAGCTGGAgCGGCTGAAGAAgGACGGCGAgGTGCGGG GCAGCATCAACAGATTCAAGACCAGCGACTACGTGAA gGAgGCCAAgCAGCTGCTGAAGGTGCAGAAGGCCTAC CACCAGCTGGACCAGAGCTTCATCGACACCTACATCG ACCTGCTGGAgACCCGGCGGACCTACTAcGAGGGcCCc GGCGAGGGCAGCCCCTTCGGCTGGAAGGACATCAAgG AgTGGTACGAGATGCTGATGGGCCACTGCACCTACTTC CCCGAGGAgCTGCGGAGCGTGAAGTACGCCTACAACG CCGACCTGTACAACGCCCTGAACGACCTGAACAAcCTg GTGATCACCAGGGACGAGAACGAGAAGCTGGAgTAcT ACGAGAAGTTCCAGATCATCGAGAACGTGTTCAAGCA GAAGAAGAAGCCCACCCTGAAGCAGATCGCCAAgGAg ATCCTgGTGAACGAgGAGGAcATcAAGGGCTACAGAGT GACCAGCACCGGCAAGCCCGAGTTCACCAACCTGAAG GTGTACCACGACATCAAGGACATcACCGCCCGGAAgG AGATcATcGAGAACGCCGAGCTGCTGGAcCAGATcGCC AAGATCCTGACCATCTACCAGAGCAGCGAGGACATCC AGGAgGAgCTGACCAAcCTGAACTCCGAGCTGACCCAG GAgGAGATCGAGCAGATCagcAAcCTGAAGGGCTAcAC CGGCACCCACAACCTGAGCCTGAAGGCCATCAACCTG ATCCTGGACGAGCTGTGGCACACCAACGACAACCAGA TCGCcATCTTCAACCGGCTGAAGCTGGTGCCCAAGAA GGTGGACCTGTCCCAGCAGAAgGAGATCCCCACCACC CTGGTGGACGACTTCATCCTGAGCCCCGTgGTGAAGA GAAGCTTCATCCAGAGCATCAAgGTGATCAACGCCAT CATCAAGAAGTACGGCCTGCCCAACGACATCATcATC GAGCTGGCCCGgGAGAAGAACTCCAAGGACGCCCAGA AgATGATCAACGAGATGCAGAAGCGGAACCGGCAGACCAACGAGCGGATCGAGGAgATCATCCGGACCACCGGC AAgGAGAACGCCAAGTACCTGATCGAGAAGATCAAGC TGCACGACATGCAGGAgGGCAAGTGCCTGTACAGCCT GGAgGCCATCCCcCTGGAgGAcCTGCTGAACAACCCCT TCAACTAcGAGGTGGACCACATCATCCCCAGAAGCGT GTCCTTCGACAACAGCTTCAACAACAAGGTGCTgGTG AAGCAGGAgGAgGCCAGCAAGAAGGGCAACCGGACCC CcTTCCAGTACCTGAGCAGCAGCGACAGCAAGATCAG CTACGAgACCTTCAAGAAGCACATCCTGAAcCTGGCCA AGGGCAAGGGCAGAATCAGCAAGACCAAGAAgGAGT AcCTGCTGGAgGAgCGGGACATCAACAGGTTCTCCGTG CAGAAgGACTTCATCAACCGGAACCTGGTGGAcACCA GATACGCCACCAGAGGCCTGATGAACCTGCTGCGGAG CTACTTCAGAGTGAACAACCTGGACGTGAAgGTGAAG TCCATCAAcGGCGGCTTCACCAGCTTcCTGCGGCGGAA GTGGAAGTTcAAGAAgGAGCGGAACAAGGGcTACAAG CACCACGCCGAGGACGCCCTGATCATcGCCAACGCCG AcTTCATCTTCAAgGAGTGGAAGAAgCTGGACAAGGCC AAgAAgGTGATGGAgAACCAGATGTTCGAGGAgAAGC AGGCCGAGAGCATGCCCGAGATCGAgACCGAGCAGGA GTACAAgGAGATCTTCATCACCCCCCACCAGATCAAG CACATcAAGGACTTCAAGGACTACAAGTACAGCCACC GGGTGGACAAGAAGCCcAAcAGAGAGCTGATcAACGA CACCCTGTACTCCACCCGGAAGGACGACAAGGGCAAC ACCCTGATCGTGAACAAcCTGAACGGCCTGTACGACA AGGACAAcGACAAGCTGAAgAAGCTGATCAACAAGAG CCCCGAgAAGCTGCTGATGTACCACCACGACCCCCAG ACCTACCAGAAgCTGAAGCTGATcATGGAgCAGTACGG CGACGAGAAGAAcCCCCTGTACAAGTACTACGAGGAg ACCGGcAACTACCTGACCAAGTACTCCAAgAAGGACA ACGGCCCCGTGATCAAGAAGATcAAGTAcTACGGCAA CAAgCTGAACGCCCAcCTGGACATCACCGACGACTACC CCAACAGCAGAAACAAGGTgGTGAAGCTGTCCCTGAA GCCCTACAGATTCGACGTGTACCTGGACAAcGGCGTGT ACAAGTTCGTGACCGTGAAGAAcCTGGAcGTGATCAAg AAgGAgAACTACTACGAgGTGAAcAGCAAGTGCTAcGA GGAgGCcAAGAAGCTGAAGAAGATCAGCAACCAGGCC GAGTTcATCGCCTCCTTCTACAACAACGAcCTGATCAA GATCAACGGCGAGCTGTAcAGAGTGATCGGCGTGAAC AACGACCTGCTGAACCGGATCGAgGTGAACATGATCG ACATCACCTACCGCGAGTACCTGGAgAACATGAACGA CAAGAGGCCCCCCAGGATCATcAAGACcATCGCCTCCA AGACCCAGAGCATcAAGAAGTACAGCACcGACATcCTG GGCAACCTGTAcGAgGTGAAgTCcAAGAAGCACCCcCA GATCATCAAgAAGGGCGGCACCGGCGGCCCCAAGAAG AAGAGGAAGGTGGGCCGGGCCCTGGAGCCCGAGAAGATCATCGGCGCCACCGACTCCTGCGGCGACCTGATGT TCCTGATGAAGTGGAAGGACACCGACGAGGCCGACCT GGTGCTGGCCAAGGAGGCCAACGTGAAGTGCCCCCAG ATCGTGATCGCCTTCTACGAGGAGCGGCTGACCTGGC ACGCCTACCCcGAGGAcGCcGAgAACAAgGAGAAgGAg ACcGCcAAGAGCTACCCCTACGACGTGCCCGACTACGC CTGATATTTGTGAAATTTGTGATGCTATTGCTTTATTT GTAACCATCTAGCTTTATTTGTGAAATTTGTGATGCTA TTGCTTTATTTGTAACCATTTTATTTGTGAAATTTGTGA TGCTATTGCTTTATTTGTAACCATTATAAGCTGCAATA AACAAGTTAACAACAACAATTGCATTCATTTTATGTTT CAGGTTCAGGGGGAGATGTGGGAGGTTTTTTAAAGCG GGAGGGCCTATTTCCCATGATTCCTTCATATTTGCATA TACGATACAAGGCTGTTAGAGAGATAATTAGAATTAA TTTGACTGTAAACACAAAGATATTAGTACAAAATACG TGACGTAGAAAGTAATAATTTCTTGGGTAGTTTGCAGT TTTAAAATTATGTTTTAAAATGGACTATCATATGCTTA CCGTAACTTGAAAGTATTTCGATTTCTTGGCTTTATAT ATCTTGTGGAAAGGACGaaacaccgNNNNNNNNNNNNNN NNNNNNNGTTTAAGTACTCTGTGCTGGAAACAGCACA GAATCTACTTAAACAAGGCAAAATGCCGTGTTTATCT CGTCAACTTGTTGGCGAGATTTTTTTggtaccggaccg |
| 51. | All-in-one CLH-22 (human CKM-HP1α) | AAV Skeletal Muscle Vector | acgcgtgatatcggacacccgagaCgccCggttataattaaccAGGACACGT GGCGAccccccccaacacctgcCcgaCctctaaaaataacTcCTggacacccg agaCgccCggttataattaaccAGGACACGTGGCGAccccccccaacacc tgcCcgaCctctaaaaataacTcCTggacacccgagaCgccCggttataattaacc AGGACACGTGGCGAccccccccaacacctgcCcgaCctctaaaaataacc cctccctggggacaAcccctccCAgcCaAtAGcacAGCCtaggTCccCcta tataaGGccaCggCTGCTGGCCCTTCCTCATTCTCAGTGTCA CCTCCAGGATACAGACAGCCCCCCTTCAGCCCAGCCC GCCACCATGGCCCCCAAGAAGAAGAGGAAGGTGGAG GCCAGCAAGCGGAACTACATCCTGGGCCTGGCCATCG GCATCACCAGCGTGGGCTACGGCATCATCGACTACGA GACcCGGGACGTGATCGAcGCCGGCGTGCGGCTGTTCA AgGAGGCCAACGTGGAgAACAACGAGGGCAGGCGGA GCAAGAGAGGCGCCAGAAGGCTGAAGCGGCGGAGGC GGCAcAGAATCCAGAGAGTGAAGAAGCTGCTGTTCGA CTACAACCTGCTGACCGACCACAGCGAGCTGAGCGGC ATCAACCCCTACGAGGCCAGAGTGAAGGGCCTGAGCC AGAAGCTGAGCGAGGAgGAGTTCagcGCCGCCCTGCTG CACCTGGCCAAGAGAAGAGGCGTGCACAACGTGAAC GAGGTGGAgGAGGACACCGGCAACGAGCTGTCCACCA AgGAGCAGATCAGCCGGAACAGCAAGGCCCTGGAgGA GAAgTACGTGGCCGAgCTGCAGCTGGAgCGGCTGAAG AAgGACGGCGAgGTGCGGGGCAGCATCAACAGATTCA AGACCAGCGACTACGTGAAgGAgGCCAAgCAGCTGCTGAAGGTGCAGAAGGCCTACCACCAGCTGGACCAGAG CTTCATCGACACCTACATCGACCTGCTGGAgACCCGGC GGACCTACTAcGAGGGcCCcGGCGAGGGCAGCCCCTTC GGCTGGAAGGACATCAAgGAgTGGTACGAGATGCTGA TGGGCCACTGCACCTACTTCCCCGAGGAgCTGCGGAG CGTGAAGTACGCCTACAACGCCGACCTGTACAACGCC CTGAACGACCTGAACAAcCTgGTGATCACCAGGGACG AGAACGAGAAGCTGGAgTAcTACGAGAAGTTCCAGAT CATCGAGAACGTGTTCAAGCAGAAGAAGAAGCCCACC CTGAAGCAGATCGCCAAgGAgATCCTgGTGAACGAgGA GGAcATcAAGGGCTACAGAGTGACCAGCACCGGCAAG CCCGAGTTCACCAACCTGAAGGTGTACCACGACATCA AGGACATcACCGCCCGGAAgGAGATcATcGAGAACGCC GAGCTGCTGGAcCAGATcGCCAAGATCCTGACCATCTA CCAGAGCAGCGAGGACATCCAGGAgGAgCTGACCAAc CTGAACTCCGAGCTGACCCAGGAgGAGATCGAGCAGA TCagcAAcCTGAAGGGCTAcACCGGCACCCACAACCTGA GCCTGAAGGCCATCAACCTGATCCTGGACGAGCTGTG GCACACCAACGACAACCAGATCGCcATCTTCAACCGG CTGAAGCTGGTGCCCAAGAAGGTGGACCTGTCCCAGC AGAAgGAGATCCCCACCACCCTGGTGGACGACTTCAT CCTGAGCCCCGTgGTGAAGAGAAGCTTCATCCAGAGC ATCAAgGTGATCAACGCCATCATCAAGAAGTACGGCC TGCCCAACGACATCATcATCGAGCTGGCCCGgGAGAA GAACTCCAAGGACGCCCAGAAgATGATCAACGAGATG CAGAAGCGGAACCGGCAGACCAACGAGCGGATCGAG GAgATCATCCGGACCACCGGCAAgGAGAACGCCAAGT ACCTGATCGAGAAGATCAAGCTGCACGACATGCAGGA gGGCAAGTGCCTGTACAGCCTGGAgGCCATCCCcCTGG AgGAcCTGCTGAACAACCCCTTCAACTAcGAGGTGGAC CACATCATCCCCAGAAGCGTGTCCTTCGACAACAGCT TCAACAACAAGGTGCTgGTGAAGCAGGAgGAgGCCAG CAAGAAGGGCAACCGGACCCCcTTCCAGTACCTGAGC AGCAGCGACAGCAAGATCAGCTACGAgACCTTCAAGA AGCACATCCTGAAcCTGGCCAAGGGCAAGGGCAGAAT CAGCAAGACCAAGAAgGAGTAcCTGCTGGAgGAgCGG GACATCAACAGGTTCTCCGTGCAGAAgGACTTCATCA ACCGGAACCTGGTGGAcACCAGATACGCCACCAGAGG CCTGATGAACCTGCTGCGGAGCTACTTCAGAGTGAAC AACCTGGACGTGAAgGTGAAGTCCATCAAcGGCGGCTT CACCAGCTTcCTGCGGCGGAAGTGGAAGTTcAAGAAgG AGCGGAACAAGGGcTACAAGCACCACGCCGAGGACGC CCTGATCATcGCCAACGCCGAcTTCATCTTCAAgGAGT GGAAGAAgCTGGACAAGGCCAAgAAgGTGATGGAgAA CCAGATGTTCGAGGAgAAGCAGGCCGAGAGCATGCCC GAGATCGAgACCGAGCAGGAGTACAAgGAGATCTTCATCACCCCCCACCAGATCAAGCACATcAAGGACTTCAA GGACTACAAGTACAGCCACCGGGTGGACAAGAAGCCc AAcAGAGAGCTGATcAACGACACCCTGTACTCCACCCG GAAGGACGACAAGGGCAACACCCTGATCGTGAACAAc CTGAACGGCCTGTACGACAAGGACAAcGACAAGCTGA AgAAGCTGATCAACAAGAGCCCCGAgAAGCTGCTGAT GTACCACCACGACCCCCAGACCTACCAGAAgCTGAAG CTGATcATGGAgCAGTACGGCGACGAGAAGAAcCCCCT GTACAAGTACTACGAGGAgACCGGcAACTACCTGACC AAGTACTCCAAgAAGGACAACGGCCCCGTGATCAAGA AGATcAAGTAcTACGGCAACAAgCTGAACGCCCAcCTG GACATCACCGACGACTACCCCAACAGCAGAAACAAG GTgGTGAAGCTGTCCCTGAAGCCCTACAGATTCGACGT GTACCTGGACAAcGGCGTGTACAAGTTCGTGACCGTG AAGAAcCTGGAcGTGATCAAgAAgGAgAACTACTACGA gGTGAAcAGCAAGTGCTAcGAGGAgGCcAAGAAGCTGA AGAAGATCAGCAACCAGGCCGAGTTcATCGCCTCCTTC TACAACAACGAcCTGATCAAGATCAACGGCGAGCTGT AcAGAGTGATCGGCGTGAACAACGACCTGCTGAACCG GATCGAgGTGAACATGATCGACATCACCTACCGCGAG TACCTGGAgAACATGAACGACAAGAGGCCCCCCAGGA TCATcAAGACcATCGCCTCCAAGACCCAGAGCATcAAG AAGTACAGCACcGACATcCTGGGCAACCTGTAcGAgGT GAAgTCcAAGAAGCACCCcCAGATCATCAAgAAGGGCG GCACCGGCGGCCCCAAGAAGAAGAGGAAGGTGGGCC GGGCCCTGGAGCCCGAGAAGATCATCGGCGCCACCGA CTCCTGCGGCGACCTGATGTTCCTGATGAAGTGGAAG GACACCGACGAGGCCGACCTGGTGCTGGCCAAGGAG GCCAACGTGAAGTGCCCCCAGATCGTGATCGCCTTCT ACGAGGAGCGGCTGACCTGGCACGCCTACCCcGAGGA cGCcGAgAACAAgGAGAAgGAgACcGCcAAGAGCTACCC CTACGACGTGCCCGACTACGCCTGATATTTGTGAAATT TGTGATGCTATTGCTTTATTTGTAACCATCTAGCTTTAT TTGTGAAATTTGTGATGCTATTGCTTTATTTGTAACCA TTTTATTTGTGAAATTTGTGATGCTATTGCTTTATTTGT AACCATTATAAGCTGCAATAAACAAGTTAACAACAAC AATTGCATTCATTTTATGTTTCAGGTTCAGGGGGAGAT GTGGGAGGTTTTTTAAAGCGGGAGGGCCTATTTCCCA TGATTCCTTCATATTTGCATATACGATACAAGGCTGTT AGAGAGATAATTAGAATTAATTTGACTGTAAACACAA AGATATTAGTACAAAATACGTGACGTAGAAAGTAATA ATTTCTTGGGTAGTTTGCAGTTTTAAAATTATGTTTTA AAATGGACTATCATATGCTTACCGTAACTTGAAAGTA TTTCGATTTCTTGGCTTTATATATCTTGTGGAAAGGAC GaaacaccgNNNNNNNNNNNNNNNNNNNNNGTTTAAGTA CTCTGTGCTGGAAACAGCACAGAATCTACTTAAACAAGGCAAAATGCCGTGTTTATCTCGTCAACTTGTTGGCGA GATTTTTTTggtaccggaccg |
| 52. | All-in-one CLH-23 (mouse CKM-HP1γ) | AAV Skeletal Muscle Vector | acgcgtgatatcggacacccgagatgcctggttataattaacccAGACATGTGG CTGccccccccaacacctgctgcgagctctaaaaataaccctgggacacccgagatg cctggttataattaacccAGACATGTGGCTGccccccccaacacctgctgcga gctctaaaaataaccctgggacacccgagatgcctggttataattaacccAGACAT GTGGCTGccccccccaacacctgctgcgagctctaaaaataacccctccctgggg acagcccctcctggctagtcacaccctgtaggctcctctatataacccaggggcacaggg gctgcccTCATTCTACCACCACCTCCACAGCACAGACAGA CACTCAGGAGCCAGCCAGCGCCACCATGGCCCCCAAG AAGAAGAGGAAGGTGGAGGCCAGCAAGCGGAACTAC ATCCTGGGCCTGGCCATCGGCATCACCAGCGTGGGCT ACGGCATCATCGACTACGAGACcCGGGACGTGATCGA cGCCGGCGTGCGGCTGTTCAAgGAGGCCAACGTGGAgA ACAACGAGGGCAGGCGGAGCAAGAGAGGCGCCAGAA GGCTGAAGCGGCGGAGGCGGCAcAGAATCCAGAGAGT GAAGAAGCTGCTGTTCGACTACAACCTGCTGACCGAC CACAGCGAGCTGAGCGGCATCAACCCCTACGAGGCCA GAGTGAAGGGCCTGAGCCAGAAGCTGAGCGAGGAgG AGTTCagcGCCGCCCTGCTGCACCTGGCCAAGAGAAGA GGCGTGCACAACGTGAACGAGGTGGAgGAGGACACCG GCAACGAGCTGTCCACCAAgGAGCAGATCAGCCGGAA CAGCAAGGCCCTGGAgGAGAAgTACGTGGCCGAgCTG CAGCTGGAgCGGCTGAAGAAgGACGGCGAgGTGCGGG GCAGCATCAACAGATTCAAGACCAGCGACTACGTGAA gGAgGCCAAgCAGCTGCTGAAGGTGCAGAAGGCCTAC CACCAGCTGGACCAGAGCTTCATCGACACCTACATCG ACCTGCTGGAgACCCGGCGGACCTACTAcGAGGGcCCc GGCGAGGGCAGCCCCTTCGGCTGGAAGGACATCAAgG AgTGGTACGAGATGCTGATGGGCCACTGCACCTACTTC CCCGAGGAgCTGCGGAGCGTGAAGTACGCCTACAACG CCGACCTGTACAACGCCCTGAACGACCTGAACAAcCTg GTGATCACCAGGGACGAGAACGAGAAGCTGGAgTAcT ACGAGAAGTTCCAGATCATCGAGAACGTGTTCAAGCA GAAGAAGAAGCCCACCCTGAAGCAGATCGCCAAgGAg ATCCTgGTGAACGAgGAGGAcATcAAGGGCTACAGAGT GACCAGCACCGGCAAGCCCGAGTTCACCAACCTGAAG GTGTACCACGACATCAAGGACATcACCGCCCGGAAgG AGATcATcGAGAACGCCGAGCTGCTGGAcCAGATcGCC AAGATCCTGACCATCTACCAGAGCAGCGAGGACATCC AGGAgGAgCTGACCAAcCTGAACTCCGAGCTGACCCAG GAgGAGATCGAGCAGATCagcAAcCTGAAGGGCTAcAC CGGCACCCACAACCTGAGCCTGAAGGCCATCAACCTG ATCCTGGACGAGCTGTGGCACACCAACGACAACCAGA TCGCcATCTTCAACCGGCTGAAGCTGGTGCCCAAGAA GGTGGACCTGTCCCAGCAGAAgGAGATCCCCACCACCCTGGTGGACGACTTCATCCTGAGCCCCGTgGTGAAGA GAAGCTTCATCCAGAGCATCAAgGTGATCAACGCCAT CATCAAGAAGTACGGCCTGCCCAACGACATCATcATC GAGCTGGCCCGgGAGAAGAACTCCAAGGACGCCCAGA AgATGATCAACGAGATGCAGAAGCGGAACCGGCAGAC CAACGAGCGGATCGAGGAgATCATCCGGACCACCGGC AAgGAGAACGCCAAGTACCTGATCGAGAAGATCAAGC TGCACGACATGCAGGAgGGCAAGTGCCTGTACAGCCT GGAgGCCATCCCcCTGGAgGAcCTGCTGAACAACCCCT TCAACTAcGAGGTGGACCACATCATCCCCAGAAGCGT GTCCTTCGACAACAGCTTCAACAACAAGGTGCTgGTG AAGCAGGAgGAgGCCAGCAAGAAGGGCAACCGGACCC CcTTCCAGTACCTGAGCAGCAGCGACAGCAAGATCAG CTACGAgACCTTCAAGAAGCACATCCTGAAcCTGGCCA AGGGCAAGGGCAGAATCAGCAAGACCAAGAAgGAGT AcCTGCTGGAgGAgCGGGACATCAACAGGTTCTCCGTG CAGAAgGACTTCATCAACCGGAACCTGGTGGAcACCA GATACGCCACCAGAGGCCTGATGAACCTGCTGCGGAG CTACTTCAGAGTGAACAACCTGGACGTGAAgGTGAAG TCCATCAAcGGCGGCTTCACCAGCTTcCTGCGGCGGAA GTGGAAGTTcAAGAAgGAGCGGAACAAGGGcTACAAG CACCACGCCGAGGACGCCCTGATCATcGCCAACGCCG AcTTCATCTTCAAgGAGTGGAAGAAgCTGGACAAGGCC AAgAAgGTGATGGAgAACCAGATGTTCGAGGAgAAGC AGGCCGAGAGCATGCCCGAGATCGAgACCGAGCAGGA GTACAAgGAGATCTTCATCACCCCCCACCAGATCAAG CACATcAAGGACTTCAAGGACTACAAGTACAGCCACC GGGTGGACAAGAAGCCcAAcAGAGAGCTGATcAACGA CACCCTGTACTCCACCCGGAAGGACGACAAGGGCAAC ACCCTGATCGTGAACAAcCTGAACGGCCTGTACGACA AGGACAAcGACAAGCTGAAgAAGCTGATCAACAAGAG CCCCGAgAAGCTGCTGATGTACCACCACGACCCCCAG ACCTACCAGAAgCTGAAGCTGATcATGGAgCAGTACGG CGACGAGAAGAAcCCCCTGTACAAGTACTACGAGGAg ACCGGcAACTACCTGACCAAGTACTCCAAgAAGGACA ACGGCCCCGTGATCAAGAAGATcAAGTAcTACGGCAA CAAgCTGAACGCCCAcCTGGACATCACCGACGACTACC CCAACAGCAGAAACAAGGTgGTGAAGCTGTCCCTGAA GCCCTACAGATTCGACGTGTACCTGGACAAcGGCGTGT ACAAGTTCGTGACCGTGAAGAAcCTGGAcGTGATCAAg AAgGAgAACTACTACGAgGTGAAcAGCAAGTGCTAcGA GGAgGCcAAGAAGCTGAAGAAGATCAGCAACCAGGCC GAGTTcATCGCCTCCTTCTACAACAACGAcCTGATCAA GATCAACGGCGAGCTGTAcAGAGTGATCGGCGTGAAC AACGACCTGCTGAACCGGATCGAgGTGAACATGATCG ACATCACCTACCGCGAGTACCTGGAgAACATGAACGACAAGAGGCCCCCCAGGATCATcAAGACcATCGCCTCCA AGACCCAGAGCATcAAGAAGTACAGCACcGACATcCTG GGCAACCTGTAcGAgGTGAAgTCcAAGAAGCACCCcCA GATCATCAAgAAGGGCGGCACCGGCGGCCCCAAGAAG AAGAGGAAGGTGGGCCGGGCCctGgaCccCgaGCgGatCat CggCgccacCgacagcagCggCgaGCtgatgttCctGatgaaGtggaaGgaC AGCgaCgaggcCgacCtggtgctggcCaaGgaggcCaaCatgaagtgCccC caGatCgtGatCgcCttCtaCgaGgagCgGctGacCtggcaCAGCtgCcc CgaGgaCgaGgcCcaGTACCCCTACGACGTGCCCGACTAC GCCTGATATTTGTGAAATTTGTGATGCTATTGCTTTAT TTGTAACCATCTAGCTTTATTTGTGAAATTTGTGATGC TATTGCTTTATTTGTAACCATTTTATTTGTGAAATTTGT GATGCTATTGCTTTATTTGTAACCATTATAAGCTGCAA TAAACAAGTTAACAACAACAATTGCATTCATTTTATGT TTCAGGTTCAGGGGGAGATGTGGGAGGTTTTTTAAAG CGGGAGGGCCTATTTCCCATGATTCCTTCATATTTGCA TATACGATACAAGGCTGTTAGAGAGATAATTAGAATT AATTTGACTGTAAACACAAAGATATTAGTACAAAATA CGTGACGTAGAAAGTAATAATTTCTTGGGTAGTTTGC AGTTTTAAAATTATGTTTTAAAATGGACTATCATATGC TTACCGTAACTTGAAAGTATTTCGATTTCTTGGCTTTA TATATCTTGTGGAAAGGACGaaacaccgNNNNNNNNNNN NNNNNNNNNNGTTTAAGTACTCTGTGCTGGAAACAGC ACAGAATCTACTTAAACAAGGCAAAATGCCGTGTTTA TCTCGTCAACTTGTTGGCGAGATTTTTTTggtaccggaccg |
| 53. | All-in-one CLH-23 (human CKM-HP1γ) | AAV Skeletal Muscle Vector | acgcgtgatatcggacacccgagaCgccCggttataattaaccAGGACACGT GGCGAccccccccaacacctgcCcgaCctctaaaaataacTcCTggacacccg agaCgccCggttataattaaccAGGACACGTGGCGAccccccccaacacc tgcCcgaCctctaaaaataacTcCTggacacccgagaCgccCggttataattaacc AGGACACGTGGCGAccccccccaacacctgcCcgaCctctaaaaataacc cctccctggggacaAcccctccCAgcCaAtAGcacAGCCtaggTCccCcta tataaGGccaCggCTGCTGGCCCTTCCTCATTCTCAGTGTCA CCTCCAGGATACAGACAGCCCCCCTTCAGCCCAGCCC GCCACCATGGCCCCCAAGAAGAAGAGGAAGGTGGAG GCCAGCAAGCGGAACTACATCCTGGGCCTGGCCATCG GCATCACCAGCGTGGGCTACGGCATCATCGACTACGA GACcCGGGACGTGATCGAcGCCGGCGTGCGGCTGTTCA AgGAGGCCAACGTGGAgAACAACGAGGGCAGGCGGA GCAAGAGAGGCGCCAGAAGGCTGAAGCGGCGGAGGC GGCAcAGAATCCAGAGAGTGAAGAAGCTGCTGTTCGA CTACAACCTGCTGACCGACCACAGCGAGCTGAGCGGC ATCAACCCCTACGAGGCCAGAGTGAAGGGCCTGAGCC AGAAGCTGAGCGAGGAgGAGTTCagcGCCGCCCTGCTG CACCTGGCCAAGAGAAGAGGCGTGCACAACGTGAAC GAGGTGGAgGAGGACACCGGCAACGAGCTGTCCACCA AgGAGCAGATCAGCCGGAACAGCAAGGCCCTGGAgGAGAAgTACGTGGCCGAgCTGCAGCTGGAgCGGCTGAAG AAgGACGGCGAgGTGCGGGGCAGCATCAACAGATTCA AGACCAGCGACTACGTGAAgGAgGCCAAgCAGCTGCT GAAGGTGCAGAAGGCCTACCACCAGCTGGACCAGAG CTTCATCGACACCTACATCGACCTGCTGGAgACCCGGC GGACCTACTAcGAGGGcCCcGGCGAGGGCAGCCCCTTC GGCTGGAAGGACATCAAgGAgTGGTACGAGATGCTGA TGGGCCACTGCACCTACTTCCCCGAGGAgCTGCGGAG CGTGAAGTACGCCTACAACGCCGACCTGTACAACGCC CTGAACGACCTGAACAAcCTgGTGATCACCAGGGACG AGAACGAGAAGCTGGAgTAcTACGAGAAGTTCCAGAT CATCGAGAACGTGTTCAAGCAGAAGAAGAAGCCCACC CTGAAGCAGATCGCCAAgGAgATCCTgGTGAACGAgGA GGAcATcAAGGGCTACAGAGTGACCAGCACCGGCAAG CCCGAGTTCACCAACCTGAAGGTGTACCACGACATCA AGGACATcACCGCCCGGAAgGAGATcATcGAGAACGCC GAGCTGCTGGAcCAGATcGCCAAGATCCTGACCATCTA CCAGAGCAGCGAGGACATCCAGGAgGAgCTGACCAAc CTGAACTCCGAGCTGACCCAGGAgGAGATCGAGCAGA TCagcAAcCTGAAGGGCTAcACCGGCACCCACAACCTGA GCCTGAAGGCCATCAACCTGATCCTGGACGAGCTGTG GCACACCAACGACAACCAGATCGCcATCTTCAACCGG CTGAAGCTGGTGCCCAAGAAGGTGGACCTGTCCCAGC AGAAgGAGATCCCCACCACCCTGGTGGACGACTTCAT CCTGAGCCCCGTgGTGAAGAGAAGCTTCATCCAGAGC ATCAAgGTGATCAACGCCATCATCAAGAAGTACGGCC TGCCCAACGACATCATcATCGAGCTGGCCCGgGAGAA GAACTCCAAGGACGCCCAGAAgATGATCAACGAGATG CAGAAGCGGAACCGGCAGACCAACGAGCGGATCGAG GAgATCATCCGGACCACCGGCAAgGAGAACGCCAAGT ACCTGATCGAGAAGATCAAGCTGCACGACATGCAGGA gGGCAAGTGCCTGTACAGCCTGGAgGCCATCCCcCTGG AgGAcCTGCTGAACAACCCCTTCAACTAcGAGGTGGAC CACATCATCCCCAGAAGCGTGTCCTTCGACAACAGCT TCAACAACAAGGTGCTgGTGAAGCAGGAgGAgGCCAG CAAGAAGGGCAACCGGACCCCcTTCCAGTACCTGAGC AGCAGCGACAGCAAGATCAGCTACGAgACCTTCAAGA AGCACATCCTGAAcCTGGCCAAGGGCAAGGGCAGAAT CAGCAAGACCAAGAAgGAGTAcCTGCTGGAgGAgCGG GACATCAACAGGTTCTCCGTGCAGAAgGACTTCATCA ACCGGAACCTGGTGGAcACCAGATACGCCACCAGAGG CCTGATGAACCTGCTGCGGAGCTACTTCAGAGTGAAC AACCTGGACGTGAAgGTGAAGTCCATCAAcGGCGGCTT CACCAGCTTcCTGCGGCGGAAGTGGAAGTTcAAGAAgG AGCGGAACAAGGGcTACAAGCACCACGCCGAGGACGC CCTGATCATcGCCAACGCCGAcTTCATCTTCAAgGAGTGGAAGAAgCTGGACAAGGCCAAgAAgGTGATGGAgAA CCAGATGTTCGAGGAgAAGCAGGCCGAGAGCATGCCC GAGATCGAgACCGAGCAGGAGTACAAgGAGATCTTCA TCACCCCCCACCAGATCAAGCACATcAAGGACTTCAA GGACTACAAGTACAGCCACCGGGTGGACAAGAAGCCc AAcAGAGAGCTGATcAACGACACCCTGTACTCCACCCG GAAGGACGACAAGGGCAACACCCTGATCGTGAACAAc CTGAACGGCCTGTACGACAAGGACAAcGACAAGCTGA AgAAGCTGATCAACAAGAGCCCCGAgAAGCTGCTGAT GTACCACCACGACCCCCAGACCTACCAGAAgCTGAAG CTGATcATGGAgCAGTACGGCGACGAGAAGAAcCCCCT GTACAAGTACTACGAGGAgACCGGcAACTACCTGACC AAGTACTCCAAgAAGGACAACGGCCCCGTGATCAAGA AGATcAAGTAcTACGGCAACAAgCTGAACGCCCAcCTG GACATCACCGACGACTACCCCAACAGCAGAAACAAG GTgGTGAAGCTGTCCCTGAAGCCCTACAGATTCGACGT GTACCTGGACAAcGGCGTGTACAAGTTCGTGACCGTG AAGAAcCTGGAcGTGATCAAgAAgGAgAACTACTACGA gGTGAAcAGCAAGTGCTAcGAGGAgGCcAAGAAGCTGA AGAAGATCAGCAACCAGGCCGAGTTcATCGCCTCCTTC TACAACAACGAcCTGATCAAGATCAACGGCGAGCTGT AcAGAGTGATCGGCGTGAACAACGACCTGCTGAACCG GATCGAgGTGAACATGATCGACATCACCTACCGCGAG TACCTGGAgAACATGAACGACAAGAGGCCCCCCAGGA TCATcAAGACcATCGCCTCCAAGACCCAGAGCATcAAG AAGTACAGCACcGACATcCTGGGCAACCTGTAcGAgGT GAAgTCcAAGAAGCACCCcCAGATCATCAAgAAGGGCG GCACCGGCGGCCCCAAGAAGAAGAGGAAGGTGGGCC GGGCCctGgaCccCgaGCgGatCatCggCgccacCgacagcagCggCg aGCtgatgttCctGatgaaGtggaaGgaCAGCgaCgaggcCgacCtggtgct ggcCaaGgaggcCaaCatgaagtgCccCcaGatCgtGatCgcCttCtaCgaG gagCgGctGacCtggcaCAGCtgCccCgaGgaCgaGgcCcaGTACC CCTACGACGTGCCCGACTACGCCTGATATTTGTGAAAT TTGTGATGCTATTGCTTTATTTGTAACCATCTAGCTTTA TTTGTGAAATTTGTGATGCTATTGCTTTATTTGTAACC ATTTTATTTGTGAAATTTGTGATGCTATTGCTTTATTTG TAACCATTATAAGCTGCAATAAACAAGTTAACAACAA CAATTGCATTCATTTTATGTTTCAGGTTCAGGGGGAGA TGTGGGAGGTTTTTTAAAGCGGGAGGGCCTATTTCCC ATGATTCCTTCATATTTGCATATACGATACAAGGCTGT TAGAGAGATAATTAGAATTAATTTGACTGTAAACACA AAGATATTAGTACAAAATACGTGACGTAGAAAGTAAT AATTTCTTGGGTAGTTTGCAGTTTTAAAATTATGTTTT AAAATGGACTATCATATGCTTACCGTAACTTGAAAGT ATTTCGATTTCTTGGCTTTATATATCTTGTGGAAAGGA CGaaacaccgNNNNNNNNNNNNNNNNNNNNNGTTTAAGTACTCTGTGCTGGAAACAGCACAGAATCTACTTAAACA AGGCAAAATGCCGTGTTTATCTCGTCAACTTGTTGGCG AGATTTTTTTtaccaccg |
| 54. | All-in-one CLH-26 (mouse CKM-SET) | AAV Skeletal Muscle Vector | acgcgtgatatcggacacccgagatgcctggttataattaacccAGACATGTGG CTGccccccccaacacctgctgcgagctctaaaaataaccctgggacacccgagatg cctggttataattaacccAGACATGTGGCTGccccccccaacacctgctgcga gctctaaaaataaccctgggacacccgagatgcctggttataattaacccAGACAT GTGGCTGccccccccaacacctgctgcgagctctaaaaataacccctccctgggg acagcccctcctggctagtcacaccctgtaggctcctctatataacccaggggcacaggg gctgcccTCATTCTACCACCACCTCCACAGCACAGACAGA CACTCAGGAGCCAGCCAGCGCCACCATGGCCCCCAAG AAGAAGAGGAAGGTGGAGGCCAGCAAGCGGAACTAC ATCCTGGGCCTGGCCATCGGCATCACCAGCGTGGGCT ACGGCATCATCGACTACGAGACcCGGGACGTGATCGA cGCCGGCGTGCGGCTGTTCAAgGAGGCCAACGTGGAgA ACAACGAGGGCAGGCGGAGCAAGAGAGGCGCCAGAA GGCTGAAGCGGCGGAGGCGGCAcAGAATCCAGAGAGT GAAGAAGCTGCTGTTCGACTACAACCTGCTGACCGAC CACAGCGAGCTGAGCGGCATCAACCCCTACGAGGCCA GAGTGAAGGGCCTGAGCCAGAAGCTGAGCGAGGAgG AGTTCagcGCCGCCCTGCTGCACCTGGCCAAGAGAAGA GGCGTGCACAACGTGAACGAGGTGGAgGAGGACACCG GCAACGAGCTGTCCACCAAgGAGCAGATCAGCCGGAA CAGCAAGGCCCTGGAgGAGAAgTACGTGGCCGAgCTG CAGCTGGAgCGGCTGAAGAAgGACGGCGAgGTGCGGG GCAGCATCAACAGATTCAAGACCAGCGACTACGTGAA gGAgGCCAAgCAGCTGCTGAAGGTGCAGAAGGCCTAC CACCAGCTGGACCAGAGCTTCATCGACACCTACATCG ACCTGCTGGAgACCCGGCGGACCTACTAcGAGGGcCCc GGCGAGGGCAGCCCCTTCGGCTGGAAGGACATCAAgG AgTGGTACGAGATGCTGATGGGCCACTGCACCTACTTC CCCGAGGAgCTGCGGAGCGTGAAGTACGCCTACAACG CCGACCTGTACAACGCCCTGAACGACCTGAACAAcCTg GTGATCACCAGGGACGAGAACGAGAAGCTGGAgTAcT ACGAGAAGTTCCAGATCATCGAGAACGTGTTCAAGCA GAAGAAGAAGCCCACCCTGAAGCAGATCGCCAAgGAg ATCCTgGTGAACGAgGAGGAcATcAAGGGCTACAGAGT GACCAGCACCGGCAAGCCCGAGTTCACCAACCTGAAG GTGTACCACGACATCAAGGACATcACCGCCCGGAAgG AGATcATcGAGAACGCCGAGCTGCTGGAcCAGATcGCC AAGATCCTGACCATCTACCAGAGCAGCGAGGACATCC AGGAgGAgCTGACCAAcCTGAACTCCGAGCTGACCCAG GAgGAGATCGAGCAGATCagcAAcCTGAAGGGCTAcAC CGGCACCCACAACCTGAGCCTGAAGGCCATCAACCTG ATCCTGGACGAGCTGTGGCACACCAACGACAACCAGA TCGCcATCTTCAACCGGCTGAAGCTGGTGCCCAAGAAGGTGGACCTGTCCCAGCAGAAgGAGATCCCCACCACC CTGGTGGACGACTTCATCCTGAGCCCCGTgGTGAAGA GAAGCTTCATCCAGAGCATCAAgGTGATCAACGCCAT CATCAAGAAGTACGGCCTGCCCAACGACATCATcATC GAGCTGGCCCGgGAGAAGAACTCCAAGGACGCCCAGA AgATGATCAACGAGATGCAGAAGCGGAACCGGCAGAC CAACGAGCGGATCGAGGAgATCATCCGGACCACCGGC AAgGAGAACGCCAAGTACCTGATCGAGAAGATCAAGC TGCACGACATGCAGGAgGGCAAGTGCCTGTACAGCCT GGAgGCCATCCCcCTGGAgGAcCTGCTGAACAACCCCT TCAACTAcGAGGTGGACCACATCATCCCCAGAAGCGT GTCCTTCGACAACAGCTTCAACAACAAGGTGCTgGTG AAGCAGGAgGAgGCCAGCAAGAAGGGCAACCGGACCC CcTTCCAGTACCTGAGCAGCAGCGACAGCAAGATCAG CTACGAgACCTTCAAGAAGCACATCCTGAAcCTGGCCA AGGGCAAGGGCAGAATCAGCAAGACCAAGAAgGAGT AcCTGCTGGAgGAgCGGGACATCAACAGGTTCTCCGTG CAGAAgGACTTCATCAACCGGAACCTGGTGGAcACCA GATACGCCACCAGAGGCCTGATGAACCTGCTGCGGAG CTACTTCAGAGTGAACAACCTGGACGTGAAgGTGAAG TCCATCAAcGGCGGCTTCACCAGCTTcCTGCGGCGGAA GTGGAAGTTcAAGAAgGAGCGGAACAAGGGcTACAAG CACCACGCCGAGGACGCCCTGATCATcGCCAACGCCG AcTTCATCTTCAAgGAGTGGAAGAAgCTGGACAAGGCC AAgAAgGTGATGGAgAACCAGATGTTCGAGGAgAAGC AGGCCGAGAGCATGCCCGAGATCGAgACCGAGCAGGA GTACAAgGAGATCTTCATCACCCCCCACCAGATCAAG CACATcAAGGACTTCAAGGACTACAAGTACAGCCACC GGGTGGACAAGAAGCCcAAcAGAGAGCTGATcAACGA CACCCTGTACTCCACCCGGAAGGACGACAAGGGCAAC ACCCTGATCGTGAACAAcCTGAACGGCCTGTACGACA AGGACAAcGACAAGCTGAAgAAGCTGATCAACAAGAG CCCCGAgAAGCTGCTGATGTACCACCACGACCCCCAG ACCTACCAGAAgCTGAAGCTGATcATGGAgCAGTACGG CGACGAGAAGAAcCCCCTGTACAAGTACTACGAGGAg ACCGGcAACTACCTGACCAAGTACTCCAAgAAGGACA ACGGCCCCGTGATCAAGAAGATcAAGTAcTACGGCAA CAAgCTGAACGCCCAcCTGGACATCACCGACGACTACC CCAACAGCAGAAACAAGGTgGTGAAGCTGTCCCTGAA GCCCTACAGATTCGACGTGTACCTGGACAAcGGCGTGT ACAAGTTCGTGACCGTGAAGAAcCTGGAcGTGATCAAg AAgGAgAACTACTACGAgGTGAAcAGCAAGTGCTAcGA GGAgGCcAAGAAGCTGAAGAAGATCAGCAACCAGGCC GAGTTcATCGCCTCCTTCTACAACAACGAcCTGATCAA GATCAACGGCGAGCTGTAcAGAGTGATCGGCGTGAAC AACGACCTGCTGAACCGGATCGAgGTGAACATGATCGACATCACCTACCGCGAGTACCTGGAgAACATGAACGA CAAGAGGCCCCCCAGGATCATcAAGACcATCGCCTCCA AGACCCAGAGCATcAAGAAGTACAGCACcGACATcCTG GGCAACCTGTAcGAgGTGAAgTCcAAGAAGCACCCcCA GATCATCAAgAAGGGCGGCACCGGCGGCCCCAAGAAG AAGAGGAAGGTGGGCCGGGCCTAcGACCTgTGCATCTT CaGgACaGAcGAcGGcCGgGGCTGGGGCGTgCGgACCCT GGAGAAGATcCGgAAGAACAGCTTCGTgATGGAGTAC GTGGGcGAGATCATcACCagcGAGGAGGCcGAGCGGCG GGGCCAGATCTACGACCGgCAGGGCGCCACCTACCTgT TcGACCTGGACTACGTGGAGGACGTGTACACCGTGGAc GCCGCCTACTAcGGCAACATCagCCACTTcGTgAACCAC AGcTGcGACCCCAACCTGCAGGTGTACAACGTgTTCATc GACAACCTgGACGAGCGGCTGCCCCGcTACCCCTACGA CGTGCCCGACTACGCCTGATATTTGTGAAATTTGTGAT GCTATTGCTTTATTTGTAACCATCTAGCTTTATTTGTGA AATTTGTGATGCTATTGCTTTATTTGTAACCATTATAA GCTGCAATAAACAAGTTAACAACAACAATTGCATTCA TTTTATGTTTCAGGTTCAGGGGGAGATGTGGGAGGTTT TTTAAAGCGGGAGGGCCTATTTCCCATGATTCCTTCAT ATTTGCATATACGATACAAGGCTGTTAGAGAGATAAT TAGAATTAATTTGACTGTAAACACAAAGATATTAGTA CAAAATACGTGACGTAGAAAGTAATAATTTCTTGGGT AGTTTGCAGTTTTAAAATTATGTTTTAAAATGGACTAT CATATGCTTACCGTAACTTGAAAGTATTTCGATTTCTT GGCTTTATATATCTTGTGGAAAGGACGaaacaccgNNNNN NNNNNNNNNNNNNNNNGTTTAAGTACTCTGTGCTGGA AACAGCACAGAATCTACTTAAACAAGGCAAAATGCCG TGTTTATCTCGTCAACTTGTTGGCGAGATTTTTTTggtacc ggaccg |
| 55. | All-in-one CLH-26 (human CKM-SET) | AAV Skeletal Muscle Vector | acgcgtgatatcggacacccgagaCgccCggttataattaaccAGGACACGT GGCGAccccccccaacacctgcCcgaCctctaaaaataacTcCTggacacccg agaCgccCggttataattaaccAGGACACGTGGCGAccccccccaacacc tgcCcgaCctctaaaaataacTcCTggacacccgagaCgccCggttataattaacc AGGACACGTGGCGAccccccccaacacctgcCcgaCctctaaaaataacc cctccctggggacaAcccctccCAgcCaAtAGcacAGCCtaggTCccCcta tataaGGccaCggCTGCTGGCCCTTCCTCATTCTCAGTGTCA CCTCCAGGATACAGACAGCCCCCCTTCAGCCCAGCCC GCCACCATGGCCCCCAAGAAGAAGAGGAAGGTGGAG GCCAGCAAGCGGAACTACATCCTGGGCCTGGCCATCG GCATCACCAGCGTGGGCTACGGCATCATCGACTACGA GACcCGGGACGTGATCGAcGCCGGCGTGCGGCTGTTCA AgGAGGCCAACGTGGAgAACAACGAGGGCAGGCGGA GCAAGAGAGGCGCCAGAAGGCTGAAGCGGCGGAGGC GGCAcAGAATCCAGAGAGTGAAGAAGCTGCTGTTCGA CTACAACCTGCTGACCGACCACAGCGAGCTGAGCGGCATCAACCCCTACGAGGCCAGAGTGAAGGGCCTGAGCC AGAAGCTGAGCGAGGAgGAGTTCagcGCCGCCCTGCTG CACCTGGCCAAGAGAAGAGGCGTGCACAACGTGAAC GAGGTGGAgGAGGACACCGGCAACGAGCTGTCCACCA AgGAGCAGATCAGCCGGAACAGCAAGGCCCTGGAgGA GAAgTACGTGGCCGAgCTGCAGCTGGAgCGGCTGAAG AAgGACGGCGAgGTGCGGGGCAGCATCAACAGATTCA AGACCAGCGACTACGTGAAgGAgGCCAAgCAGCTGCT GAAGGTGCAGAAGGCCTACCACCAGCTGGACCAGAG CTTCATCGACACCTACATCGACCTGCTGGAgACCCGGC GGACCTACTAcGAGGGcCCcGGCGAGGGCAGCCCCTTC GGCTGGAAGGACATCAAgGAgTGGTACGAGATGCTGA TGGGCCACTGCACCTACTTCCCCGAGGAgCTGCGGAG CGTGAAGTACGCCTACAACGCCGACCTGTACAACGCC CTGAACGACCTGAACAAcCTgGTGATCACCAGGGACG AGAACGAGAAGCTGGAgTAcTACGAGAAGTTCCAGAT CATCGAGAACGTGTTCAAGCAGAAGAAGAAGCCCACC CTGAAGCAGATCGCCAAgGAgATCCTgGTGAACGAgGA GGAcATcAAGGGCTACAGAGTGACCAGCACCGGCAAG CCCGAGTTCACCAACCTGAAGGTGTACCACGACATCA AGGACATcACCGCCCGGAAgGAGATcATcGAGAACGCC GAGCTGCTGGAcCAGATcGCCAAGATCCTGACCATCTA CCAGAGCAGCGAGGACATCCAGGAgGAgCTGACCAAc CTGAACTCCGAGCTGACCCAGGAgGAGATCGAGCAGA TCagcAAcCTGAAGGGCTAcACCGGCACCCACAACCTGA GCCTGAAGGCCATCAACCTGATCCTGGACGAGCTGTG GCACACCAACGACAACCAGATCGCcATCTTCAACCGG CTGAAGCTGGTGCCCAAGAAGGTGGACCTGTCCCAGC AGAAgGAGATCCCCACCACCCTGGTGGACGACTTCAT CCTGAGCCCCGTgGTGAAGAGAAGCTTCATCCAGAGC ATCAAgGTGATCAACGCCATCATCAAGAAGTACGGCC TGCCCAACGACATCATcATCGAGCTGGCCCGgGAGAA GAACTCCAAGGACGCCCAGAAgATGATCAACGAGATG CAGAAGCGGAACCGGCAGACCAACGAGCGGATCGAG GAgATCATCCGGACCACCGGCAAgGAGAACGCCAAGT ACCTGATCGAGAAGATCAAGCTGCACGACATGCAGGA gGGCAAGTGCCTGTACAGCCTGGAgGCCATCCCcCTGG AgGAcCTGCTGAACAACCCCTTCAACTAcGAGGTGGAC CACATCATCCCCAGAAGCGTGTCCTTCGACAACAGCT TCAACAACAAGGTGCTgGTGAAGCAGGAgGAgGCCAG CAAGAAGGGCAACCGGACCCCcTTCCAGTACCTGAGC AGCAGCGACAGCAAGATCAGCTACGAgACCTTCAAGA AGCACATCCTGAAcCTGGCCAAGGGCAAGGGCAGAAT CAGCAAGACCAAGAAgGAGTAcCTGCTGGAgGAgCGG GACATCAACAGGTTCTCCGTGCAGAAgGACTTCATCA ACCGGAACCTGGTGGAcACCAGATACGCCACCAGAGGCCTGATGAACCTGCTGCGGAGCTACTTCAGAGTGAAC AACCTGGACGTGAAgGTGAAGTCCATCAAcGGCGGCTT CACCAGCTTcCTGCGGCGGAAGTGGAAGTTcAAGAAgG AGCGGAACAAGGGcTACAAGCACCACGCCGAGGACGC CCTGATCATcGCCAACGCCGAcTTCATCTTCAAgGAGT GGAAGAAgCTGGACAAGGCCAAgAAgGTGATGGAgAA CCAGATGTTCGAGGAgAAGCAGGCCGAGAGCATGCCC GAGATCGAgACCGAGCAGGAGTACAAgGAGATCTTCA TCACCCCCCACCAGATCAAGCACATcAAGGACTTCAA GGACTACAAGTACAGCCACCGGGTGGACAAGAAGCCc AAcAGAGAGCTGATcAACGACACCCTGTACTCCACCCG GAAGGACGACAAGGGCAACACCCTGATCGTGAACAAc CTGAACGGCCTGTACGACAAGGACAAcGACAAGCTGA AgAAGCTGATCAACAAGAGCCCCGAgAAGCTGCTGAT GTACCACCACGACCCCCAGACCTACCAGAAgCTGAAG CTGATcATGGAgCAGTACGGCGACGAGAAGAAcCCCCT GTACAAGTACTACGAGGAgACCGGcAACTACCTGACC AAGTACTCCAAgAAGGACAACGGCCCCGTGATCAAGA AGATcAAGTAcTACGGCAACAAgCTGAACGCCCAcCTG GACATCACCGACGACTACCCCAACAGCAGAAACAAG GTgGTGAAGCTGTCCCTGAAGCCCTACAGATTCGACGT GTACCTGGACAAcGGCGTGTACAAGTTCGTGACCGTG AAGAAcCTGGAcGTGATCAAgAAgGAgAACTACTACGA gGTGAAcAGCAAGTGCTAcGAGGAgGCcAAGAAGCTGA AGAAGATCAGCAACCAGGCCGAGTTcATCGCCTCCTTC TACAACAACGAcCTGATCAAGATCAACGGCGAGCTGT AcAGAGTGATCGGCGTGAACAACGACCTGCTGAACCG GATCGAgGTGAACATGATCGACATCACCTACCGCGAG TACCTGGAgAACATGAACGACAAGAGGCCCCCCAGGA TCATcAAGACcATCGCCTCCAAGACCCAGAGCATcAAG AAGTACAGCACcGACATcCTGGGCAACCTGTAcGAgGT GAAgTCcAAGAAGCACCCcCAGATCATCAAgAAGGGCG GCACCGGCGGCCCCAAGAAGAAGAGGAAGGTGGGCC GGGCCTAcGACCTgTGCATCTTCaGgACaGAcGAcGGcCG gGGCTGGGGCGTgCGgACCCTGGAGAAGATcCGgAAGA ACAGCTTCGTgATGGAGTACGTGGGcGAGATCATcACC agcGAGGAGGCcGAGCGGCGGGGCCAGATCTACGACCG gCAGGGCGCCACCTACCTgTTcGACCTGGACTACGTGG AGGACGTGTACACCGTGGAcGCCGCCTACTAcGGCAAC ATCagCCACTTcGTgAACCACAGcTGcGACCCCAACCTG CAGGTGTACAACGTgTTCATcGACAACCTgGACGAGCG GCTGCCCCGcTACCCCTACGACGTGCCCGACTACGCCT GATATTTGTGAAATTTGTGATGCTATTGCTTTATTTGT AACCATCTAGCTTTATTTGTGAAATTTGTGATGCTATT GCTTTATTTGTAACCATTATAAGCTGCAATAAACAAGT TAACAACAACAATTGCATTCATTTTATGTTTCAGGTTCAGGGGGAGATGTGGGAGGTTTTTTAAAGCGGGAGGGC CTATTTCCCATGATTCCTTCATATTTGCATATACGATA CAAGGCTGTTAGAGAGATAATTAGAATTAATTTGACT GTAAACACAAAGATATTAGTACAAAATACGTGACGTA GAAAGTAATAATTTCTTGGGTAGTTTGCAGTTTTAAAA TTATGTTTTAAAATGGACTATCATATGCTTACCGTAAC TTGAAAGTATTTCGATTTCTTGGCTTTATATATCTTGTG GAAAGGACGaaacaccgNNNNNNNNNNNNNNNNNNNNN GTTTAAGTACTCTGTGCTGGAAACAGCACAGAATCTA CTTAAACAAGGCAAAATGCCGTGTTTATCTCGTCAACT TGTTGGCGAGATTTTTTTggtaccggaccg |
| 56 | MeCP2 | AAV Vector | MAPKKKRKVEASKRNYILGLAIGITSVGYGIIDYETRDVI DAGVRLFKEANVENNEGRRSKRGARRLKRRRRHRIQRV KKLLFDYNLLTDHSELSGINPYEARVKGLSQKLSEEEFSA ALLHLAKRRGVHNVNEVEEDTGNELSTKEQISRNSKALE EKYVAELQLERLKKDGEVRGSINRFKTSDYVKEAKQLL KVQKAYHQLDQSFIDTYIDLLETRRTYYEGPGEGSPFGW KDIKEWYEMLMGHCTYFPEELRSVKYAYNADLYNALN DLNNLVITRDENEKLEYYEKFQIIENVFKQKKKPTLKQIA KEILVNEEDIKGYRVTSTGKPEFTNLKVYHDIKDITARKE IIENAELLDQIAKILTIYQSSEDIQEELTNLNSELTQEEIEQI SNLKGYTGTHNLSLKAINLILDELWHTNDNQIAIFNRLKL VPKKVDLSQQKEIPTTLVDDFILSPVVKRSFIQSIKVINAII KKYGLPNDIIIELAREKNSKDAQKMINEMQKRNRQTNER IEEIIRTTGKENAKYLIEKIKLHDMQEGKCLYSLEAIPLED LLNNPFNYEVDHIIPRSVSFDNSFNNKVLVKQEEASKKG NRTPFQYLSSSDSKISYETFKKHILNLAKGKGRISKTKKE YLLEERDINRFSVQKDFINRNLVDTRYATRGLMNLLRSY FRVNNLDVKVKSINGGFTSFLRRKWKFKKERNKGYKHH AEDALIIANADFIFKEWKKLDKAKKVMENQMFEEKQAE SMPEIETEQEYKEIFITPHQIKHIKDFKDYKYSHRVDKKP NRELINDTLYSTRKDDKGNTLIVNNLNGLYDKDNDKLK KLINKSPEKLLMYHHDPQTYQKLKLIMEQYGDEKNPLY KYYEETGNYLTKYSKKDNGPVIKKIKYYGNKLNAHLDI TDDYPNSRNKVVKLSLKPYRFDVYLDNGVYKFVTVKNL DVIKKENYYEVNSKCYEEAKKLKKISNQAEFIASFYNND LIKINGELYRVIGVNNDLLNRIEVNMIDITYREYLENMND KRPPRIIKTIASKTQSIKKYSTDILGNLYEVKSKKHPQIIKK GGTGGPKKKRKVGRAGRKPGSVVAAAAAEAKKKAVKE SSIRSVQETVLPIKKRKTRA |
| 57 | HP 1 alpha | AAV Vector | MAPKKKRKVEASKRNYILGLAIGITSVGYGIIDYETRDVI DAGVRLFKEANVENNEGRRSKRGARRLKRRRRHRIQRV KKLLFDYNLLTDHSELSGINPYEARVKGLSQKLSEEEFSA ALLHLAKRRGVHNVNEVEEDTGNELSTKEQISRNSKALE EKYVAELQLERLKKDGEVRGSINRFKTSDYVKEAKQLL KVQKAYHQLDQSFIDTYIDLLETRRTYYEGPGEGSPFGW KDIKEWYEMLMGHCTYFPEELRSVKYAYNADLYNALN DLNNLVITRDENEKLEYYEKFQIIENVFKQKKKPTLKQIA KEILVNEEDIKGYRVTSTGKPEFTNLKVYHDIKDITARKE IIENAELLDQIAKILTIYQSSEDIQEELTNLNSELTQEEIEQI SNLKGYTGTHNLSLKAINLILDELWHTNDNQIAIFNRLKL VPKKVDLSQQKEIPTTLVDDFILSPVVKRSFIQSIKVINAII KKYGLPNDIIIELAREKNSKDAQKMINEMQKRNRQTNER IEEIIRTTGKENAKYLIEKIKLHDMQEGKCLYSLEAIPLED LLNNPFNYEVDHIIPRSVSFDNSFNNKVLVKQEEASKKG NRTPFQYLSSSDSKISYETFKKHILNLAKGKGRISKTKKE YLLEERDINRFSVQKDFINRNLVDTRYATRGLMNLLRSY FRVNNLDVKVKSINGGFTSFLRRKWKFKKERNKGYKHH AEDALIIANADFIFKEWKKLDKAKKVMENQMFEEKQAE SMPEIETEQEYKEIFITPHQIKHIKDFKDYKYSHRVDKKP NRELINDTLYSTRKDDKGNTLIVNNLNGLYDKDNDKLK KLINKSPEKLLMYHHDPQTYQKLKLIMEQYGDEKNPLY KYYEETGNYLTKYSKKDNGPVIKKIKYYGNKLNAHLDI TDDYPNSRNKVVKLSLKPYRFDVYLDNGVYKFVTVKNL DVIKKENYYEVNSKCYEEAKKLKKISNQAEFIASFYNND LIKINGELYRVIGVNNDLLNRIEVNMIDITYREYLENMND KRPPRIIKTIASKTQSIKKYSTDILGNLYEVKSKKHPQIIKK GGTGGPKKKRKVGRAEPEKIIGATDSCGDLMFLMKWKD TDEADLVLAKEANVKCPQIVIAFYEERLTWHAYPEDAE NKEKETAKSA |
| 58 | HP 1 gamm a | AAV Vector | MAPKKKRKVEASKRNYILGLAIGITSVGYGIIDYETRDVI DAGVRLFKEANVENNEGRRSKRGARRLKRRRRHRIQRV KKLLFDYNLLTDHSELSGINPYEARVKGLSQKLSEEEFSA ALLHLAKRRGVHNVNEVEEDTGNELSTKEQISRNSKALE EKYVAELQLERLKKDGEVRGSINRFKTSDYVKEAKQLL KVQKAYHQLDQSFIDTYIDLLETRRTYYEGPGEGSPFGW KDIKEWYEMLMGHCTYFPEELRSVKYAYNADLYNALN DLNNLVITRDENEKLEYYEKFQIIENVFKQKKKPTLKQIA KEILVNEEDIKGYRVTSTGKPEFTNLKVYHDIKDITARKE IIENAELLDQIAKILTIYQSSEDIQEELTNLNSELTQEEIEQI SNLKGYTGTHNLSLKAINLILDELWHTNDNQIAIFNRLKL VPKKVDLSQQKEIPTTLVDDFILSPVVKRSFIQSIKVINAII KKYGLPNDIIIELAREKNSKDAQKMINEMQKRNRQTNER IEEIIRTTGKENAKYLIEKIKLHDMQEGKCLYSLEAIPLED LLNNPFNYEVDHIIPRSVSFDNSFNNKVLVKQEEASKKG NRTPFQYLSSSDSKISYETFKKHILNLAKGKGRISKTKKE YLLEERDINRFSVQKDFINRNLVDTRYATRGLMNLLRSY FRVNNLDVKVKSINGGFTSFLRRKWKFKKERNKGYKHH AEDALIIANADFIFKEWKKLDKAKKVMENQMFEEKQAE SMPEIETEQEYKEIFITPHQIKHIKDFKDYKYSHRVDKKP NRELINDTLYSTRKDDKGNTLIVNNLNGLYDKDNDKLK KLINKSPEKLLMYHHDPQTYQKLKLIMEQYGDEKNPLY KYYEETGNYLTKYSKKDNGPVIKKIKYYGNKLNAHLDI TDDYPNSRNKVVKLSLKPYRFDVYLDNGVYKFVTVKNL DVIKKENYYEVNSKCYEEAKKLKKISNQAEFIASFYNND LIKINGELYRVIGVNNDLLNRIEVNMIDITYREYLENMND KRPPRIIKTIASKTQSIKKYSTDILGNLYEVKSKKHPQIIKK GGTGGPKKKRKVGRALDPERIIGATDSSGELMFLMKWK DSDEADLVLAKEANMKCPQIVIAFYEERLTWHSCPEDEA QA |

*Sequences marked in bold are locations of the sgRNAs (see Table 2)

The experimental results are now described.

Example 1: dSaCas9-mediated recruitment of epigenetic repressors to the DUX4 promoter or exon 1 represses DUX4-fl and DUX4-FL targets in FSHD myocytes. An effective CRISPR-based FSHD therapy will require both efficient delivery of therapeutic components to skeletal muscles and long-term repression of the disease locus. To address these needs, an existing CRISPRi platform was re-engineered. Previous studies have used a dSpCas9 fused to the KRAB domain (FIG. 1A), which was sufficient for short-term inhibition in cultured cells, but not ideal for long-term silencing. Stable silencing might be directly accomplished by targeting DNA methyltransferases (DNMTs) to DUX4; however, the catalytic domains of these enzymes are much too large to fit the packaging constraints (~4.4 kb) of current AAV vectors needed for in vivo delivery. Thus, smaller epigenetic regulators and repressive domains were selected that can also effect stable silencing.

While encompassing a range of diverse functions, HP1 proteins are key mediators of heterochromatin formation. HP1α predominantly localizes to heterochromatin and HP1γ is enriched at the D4Z4 macrosatellite array in healthy myocytes and lost in FSHD. SUV39H1 is a histone methyltransferase that establishes constitutive heterochromatin at pericentric and telomeric regions. The SET domain of SUV39H1 participates in stable binding to heterochromatin and mediates H3K9 trimethylation, a repressive mark that recruits HP1. Although the SET domain contains the active site of enzymatic activity, both pre-SET and post-SET domains are required for methyltransferase activity. The methyl-CpG-binding protein MeCP2 also plays diverse roles in chromatin regulation, but its TRD binds repressive histone marks and co-repressor complexes.

To accommodate dCas9 fused to even these relatively small repressors and repressive domains in AAV vectors required minimizing current regulatory cassettes. Building on key work from the Hauschka lab (Salva, et al. (2007) Mol Ther. 15: 320-9; Himeda, et al. (2011) Methods Mol Biol. 34:1942-55), a minimized skeletal muscle regulatory cassette was designed to allow larger therapeutic components to be delivered in vivo. Starting with a CKM-based cassette, which is a modified version of three CKM enhancers upstream of the CKM promoter (Himeda, et al. (2011) Methods Mol Biol. 34:1942-55), additional space was removed between elements and the CarG and AP2 sites were deleted, which are dispensable for expression in skeletal muscle (Amacher, et al. (1993) Mol Cell Biol. 13:2753-64; Donoviel, et al. (1996) Mol Cell Biol. 16: 1649-58). This reduced the size of the regulatory cassette to 378 bp, allowing the creation of constructs containing the smaller dSaCas9 ortholog fused to epigenetic repressors that were previously too large to fit into AAV vectors. Thus, the new CRISPRi platform consists of: 1) dSaCas9 fused to one of four epigenetic repressors (HP1α, HP1γ, the MeCP2 TRD, or the SUV39H1 pre-SET, SET, and post-SET domains) under control of an FSHD-optimized regulatory cassette, and 2) an sgRNA targeting the DUX4 locus under control of the U6 promoter (FIG. 1B). While these components were initially expressed in lentiviral (LV) vectors for infection of cultured myocytes, each therapeutic cassette can be efficiently packaged in AAV vectors for in vivo use.

Single guide RNAs (sgRNAs) were designed which are compatible with the Sa protospacer adjacent motif (PAM) (NNGRRT) targeting across the DUX4 locus (Materials and Methods and FIG. 1D). For all experiments, four serial coinfections of FSHD myogenic cultures were performed as described (Himeda, et al. (2016) Mol Ther. 24:527-35). Cells were infected with various combinations of LV supernatants expressing dSaCas9 fused to each epigenetic regulator or individual sgRNAs. Cells were harvested 3 d following the final round of infection for analysis of gene expression by qRT-PCR.

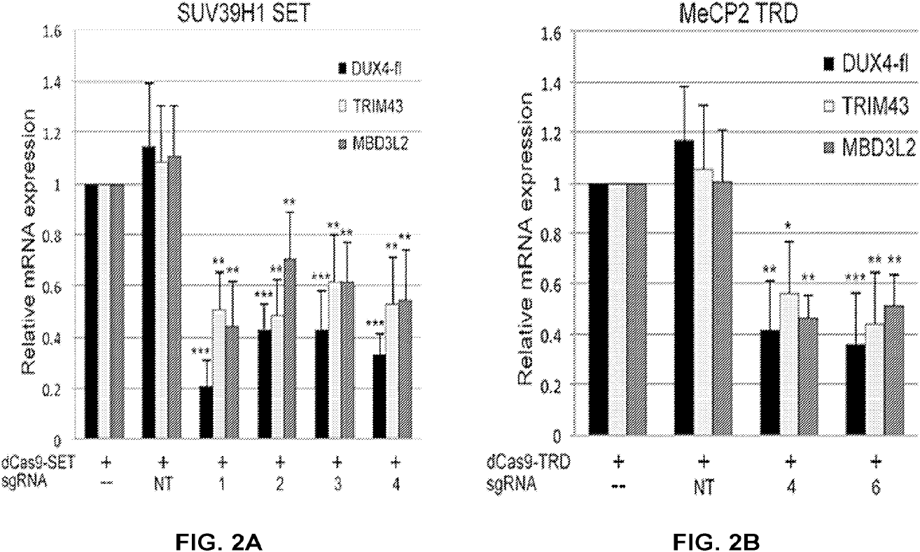

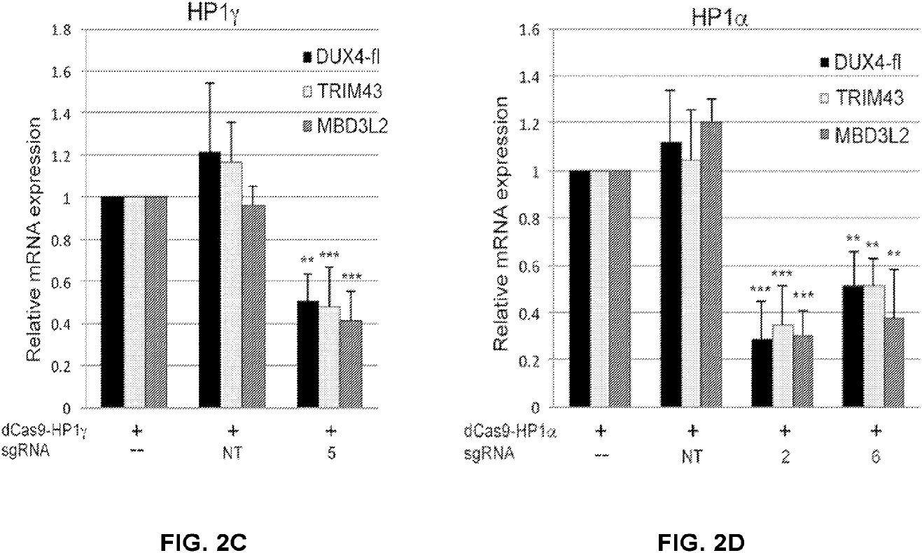

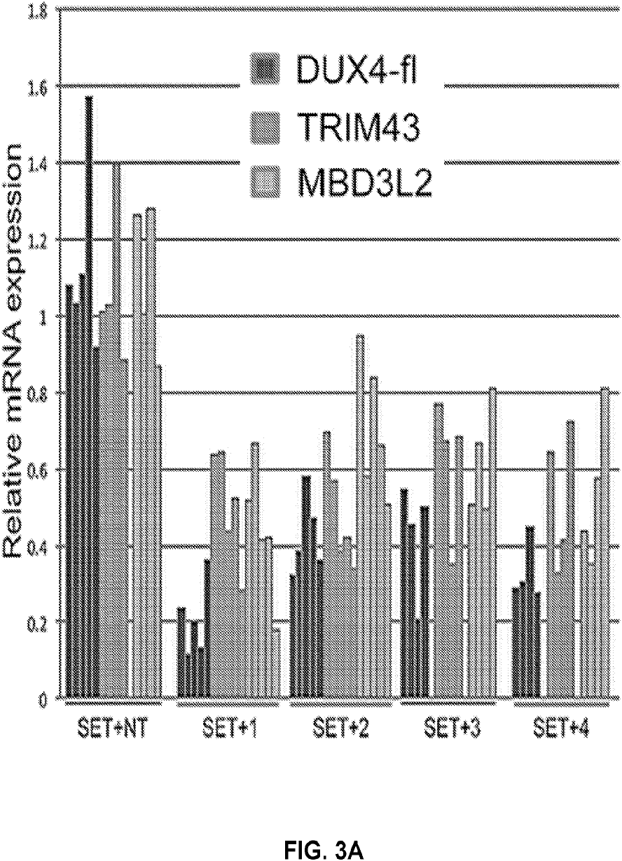

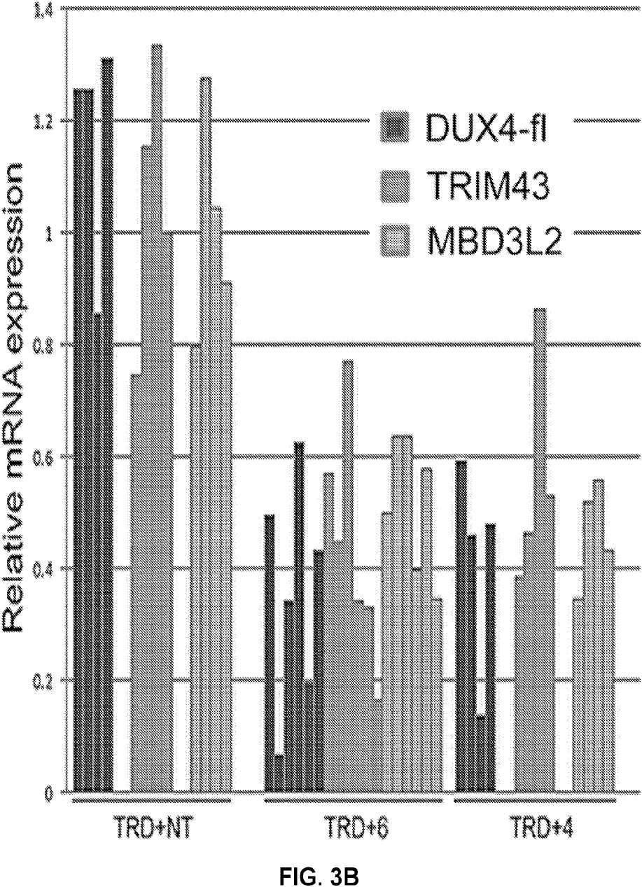

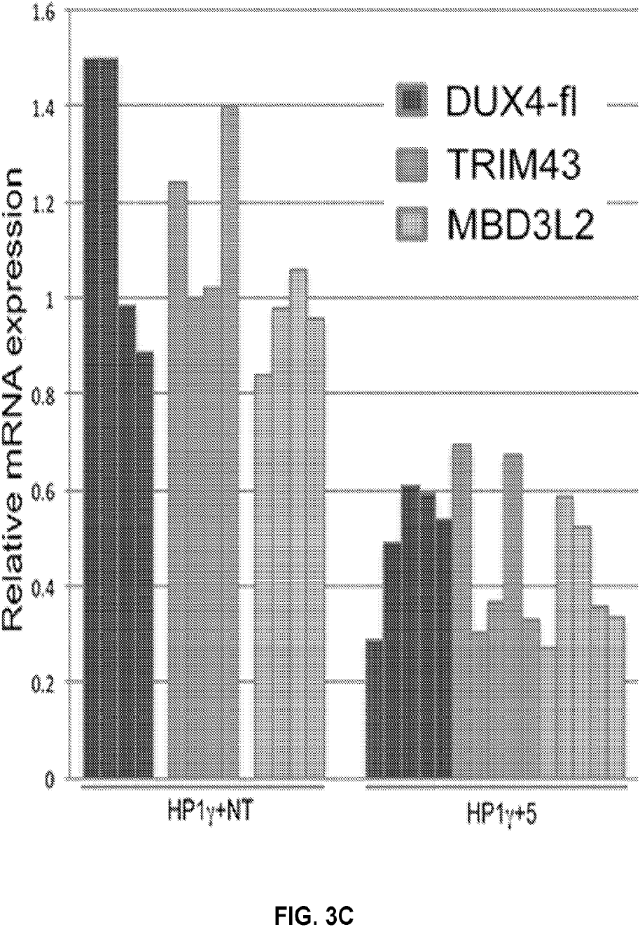

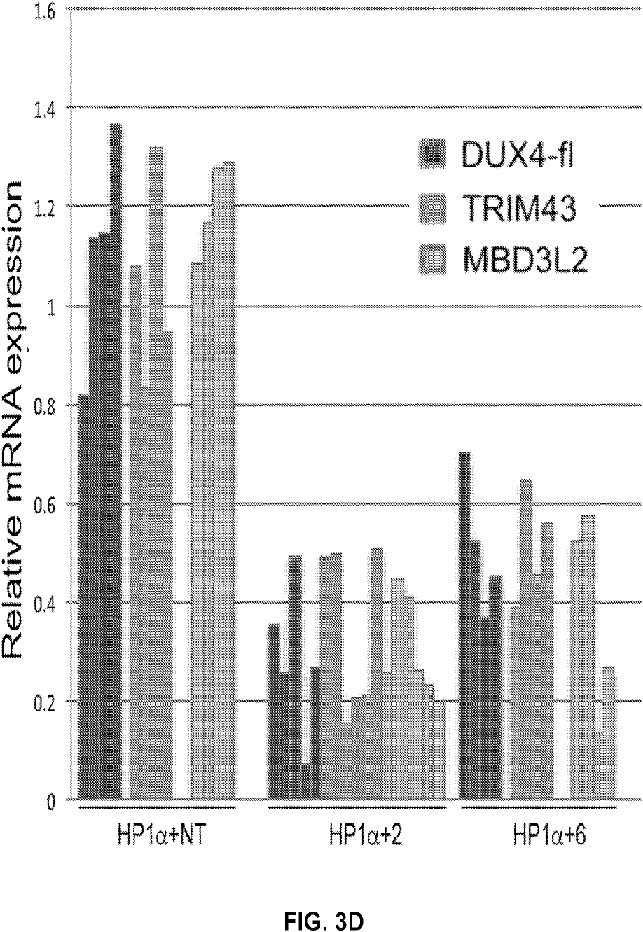

While targeting DUX4 exon 3 or the D4Z4 upstream enhancers had no effect, targeting each dSaCas9-epigenetic regulator to the DUX4 promoter or exon 1 significantly reduced levels of DUX4-fl mRNA to -30-50% of endogenous levels (FIGS. 2 and 3). As levels of DUX4-FL protein are low and difficult to assess in FSHD myocytes, DUX4-FL target gene expression was routinely assessed as a more reliable assay and relevant functional readout of DUX4 activity. Importantly, expression levels of DUX4-FL targets thought to have pathogenic consequences are significantly decreased in parallel with the reduction in DUX4-fl mRNA (FIGS. 2, 3).

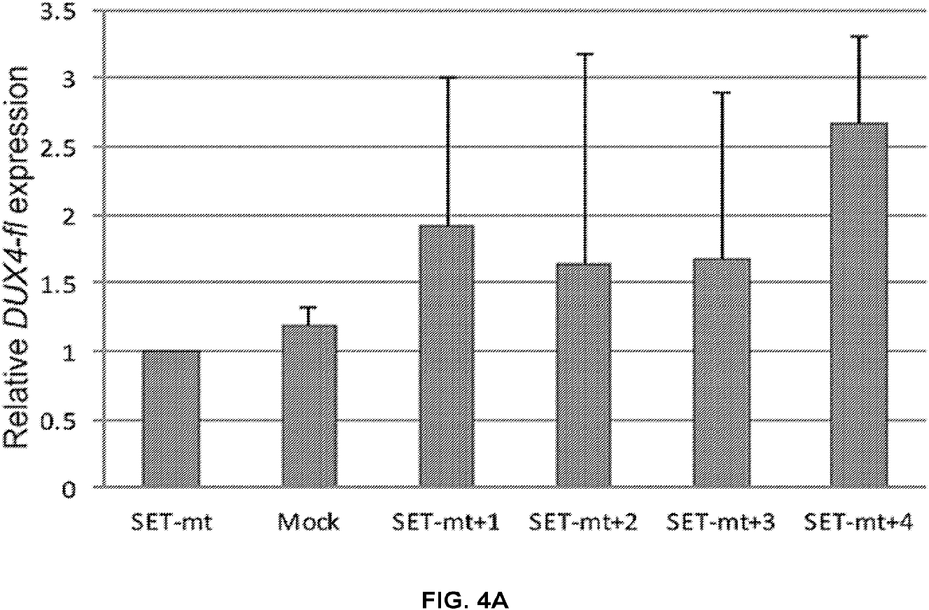

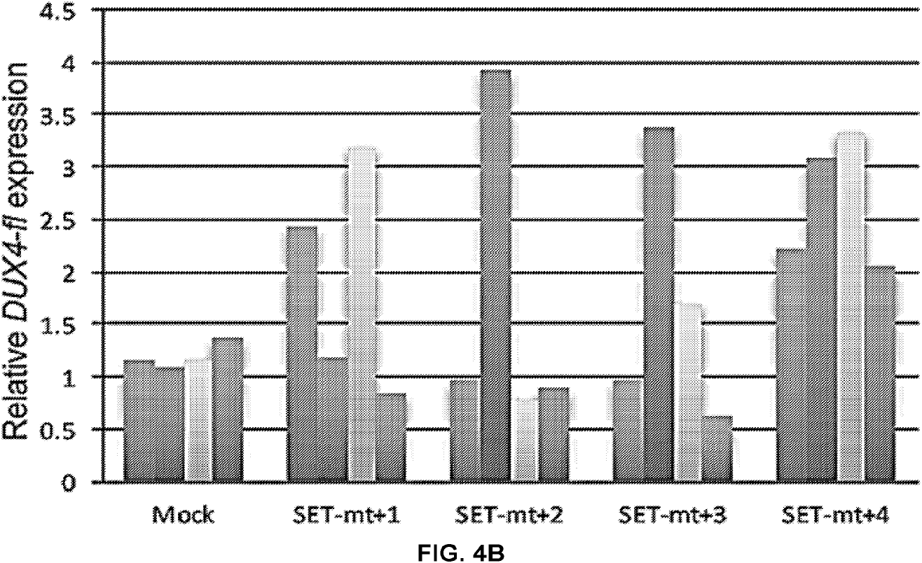

To verify that enzymatic activity of the SET domain is required for the effect on DUX4-fl, a dSaCas9-SET containing a mutation (C326A) within the SET domain was created that abolishes enzymatic activity to the DUX4 promoter/exon 1. Although the effect was highly variable, the inactive SET domain did not significantly affect levels of DUX4-fl (FIG. 4), indicating that enzymatic activity of this region is required for DUX4-fl repression.

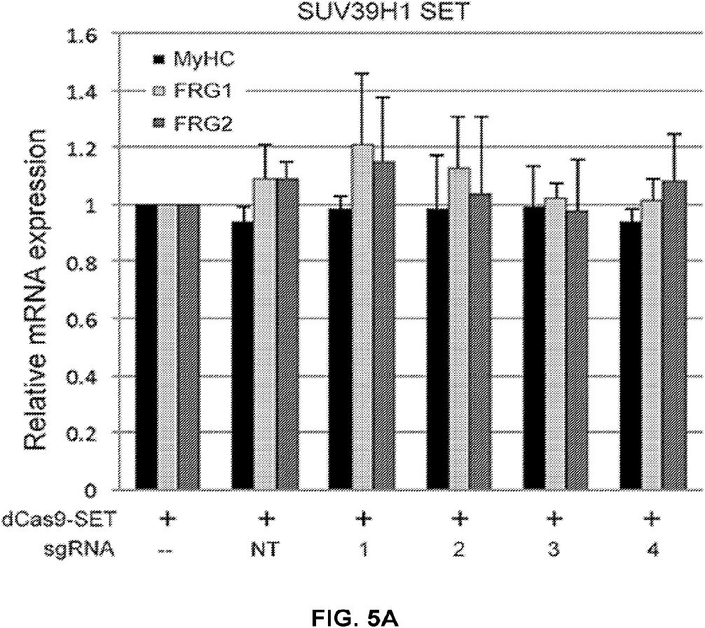

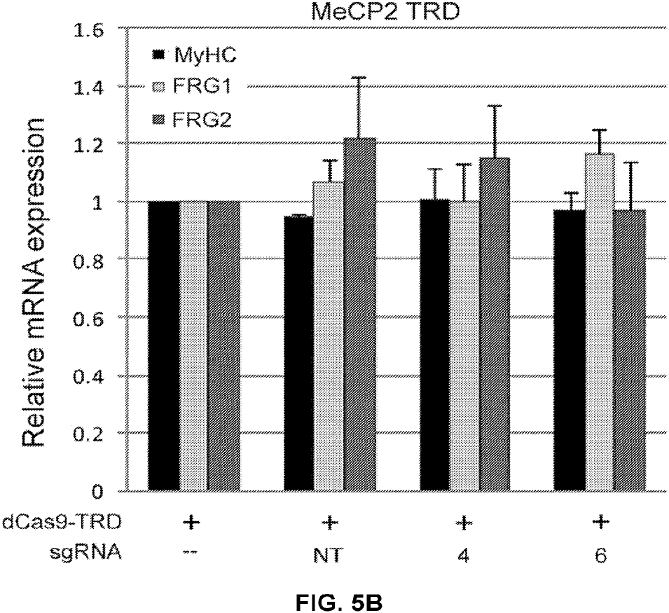

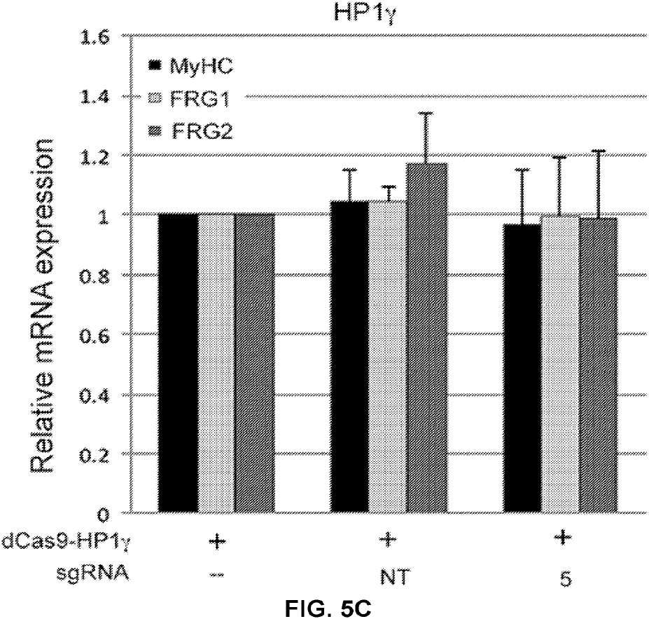

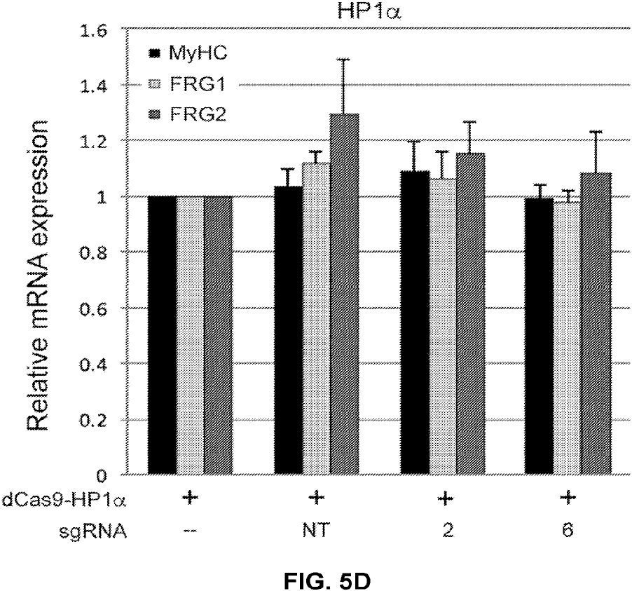

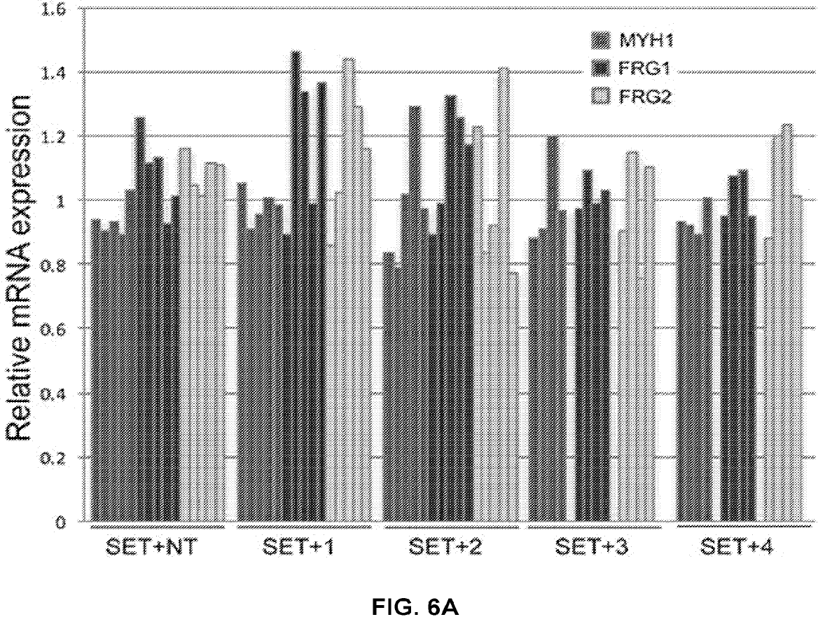

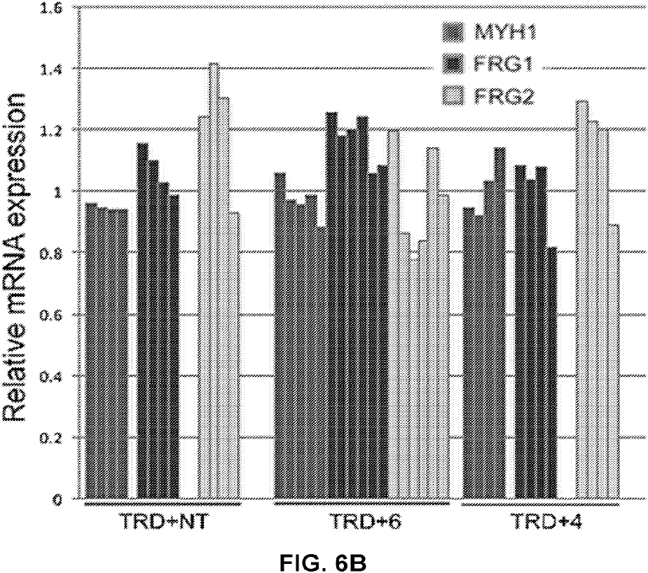

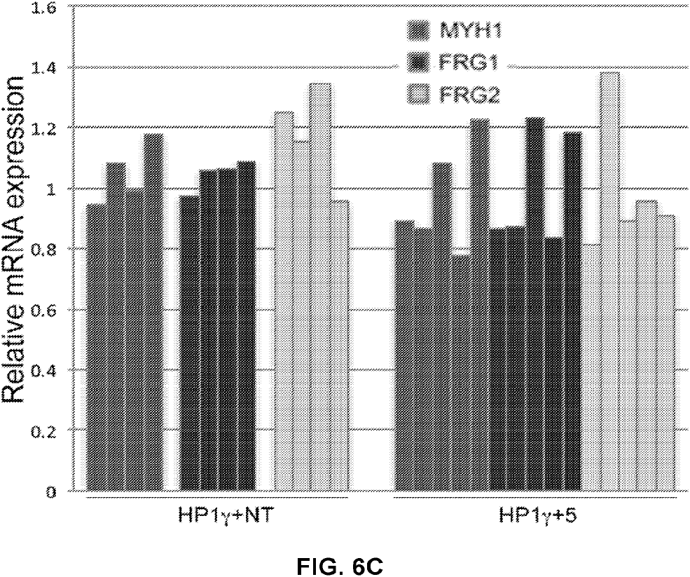

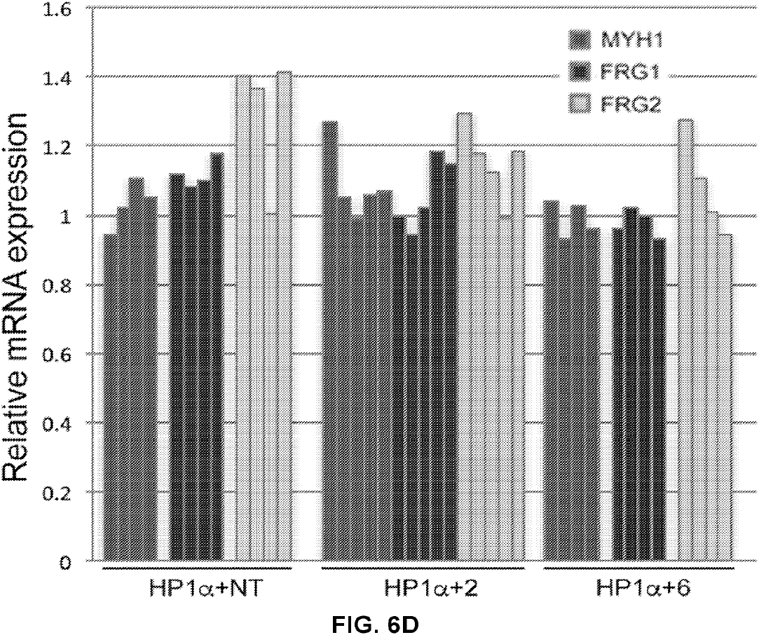

Example 2: Targeting dSaCas9-epigenetic repressors to DUX4 has no effect on MYH1, D4Z4 proximal genes, or closest-match OT genes expressed in skeletal muscle. To rule out a nonspecific effect of dSaCas9-epigenetic repressors on muscle differentiation, levels of Myosin heavy chain 1 (MYH1), a marker of terminal muscle differentiation were assessed by qRT-PCR in the cells described above. Importantly, MYH1 levels were equivalent in all cultures (FIGS. 5-6), indicating that lower levels of DUX4-fl are not due to impairment of differentiation. Expression levels of FRG1 and FRG2 were also measured. These two other FSHD candidate genes lie proximal to the D4Z4 macrosatellite. Recruitment of each dSaCas9-repressor to the DUX4 promoter/exon 1 did not reduce expression of these D4Z4 proximal genes (FIGS. 5-6).

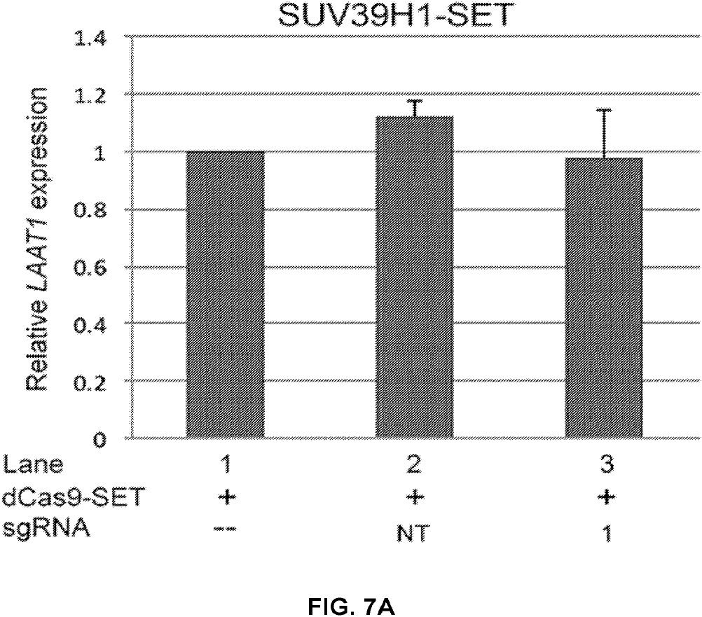

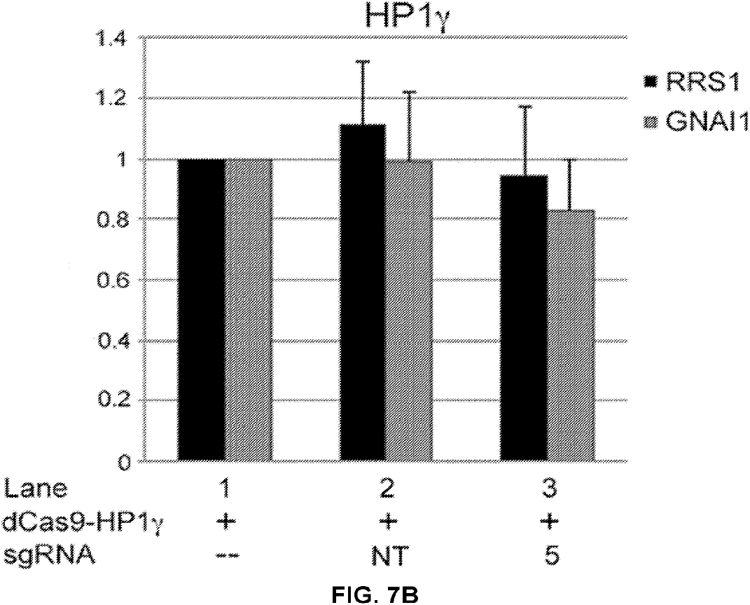

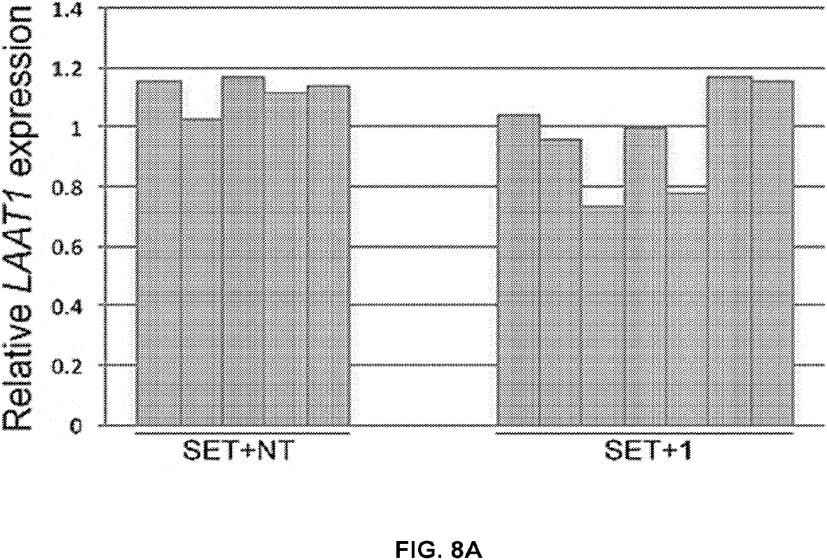

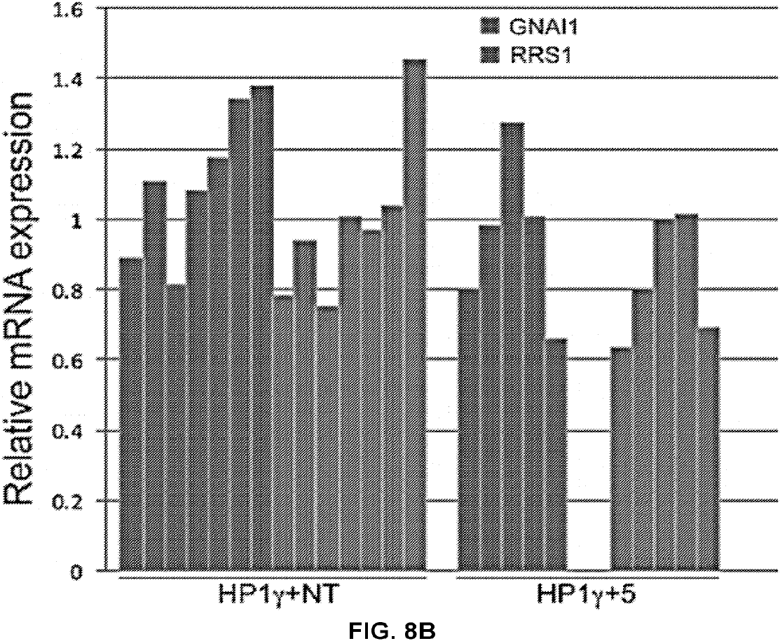

For the sgRNAs that worked best in combination with each dSaCas9-repressor, the publically available Cas-OFFinder tool (http://www.rgenome.net/cas-offinder/) was used to search for the closest-matching OT sequences in the human genome. Only sgRNAs #1 and #5 had close-matching OTs in or near genes expressed in skeletal muscle (Table 1). Intron 1 of Lysosomal amino acid transporter 1 homolog (LAAT1) contains a potential OT match to sgRNA #1. The single exon of Ribosome biogenesis regulatory protein homolog (RRS1) and the sequence 283 bp downstream of Guanine nucleotide-binding protein G(i) subunit alpha-1 isoform 1 (GNAI1) contain potential OT matches to sgRNA #5. However, in contrast to the striking reduction in DUX4-fl, targeting dSaCas9-SET with sgRNA #1 had no effect on LAAT1 expression (FIGS. 7A and 8A). Similarly, targeting dSaCas9-HP1γ with sgRNA #5 had no effect on levels of RRS1 or GNAI1 (FIGS. 7B and 8B).

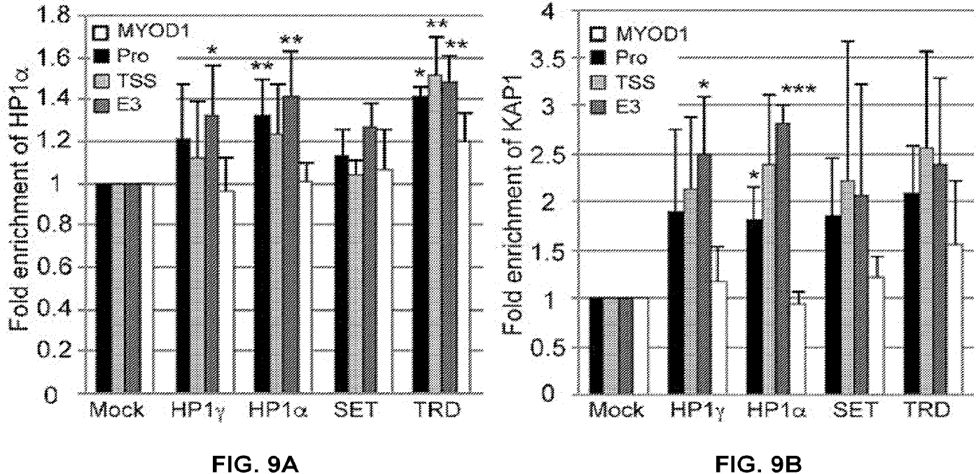

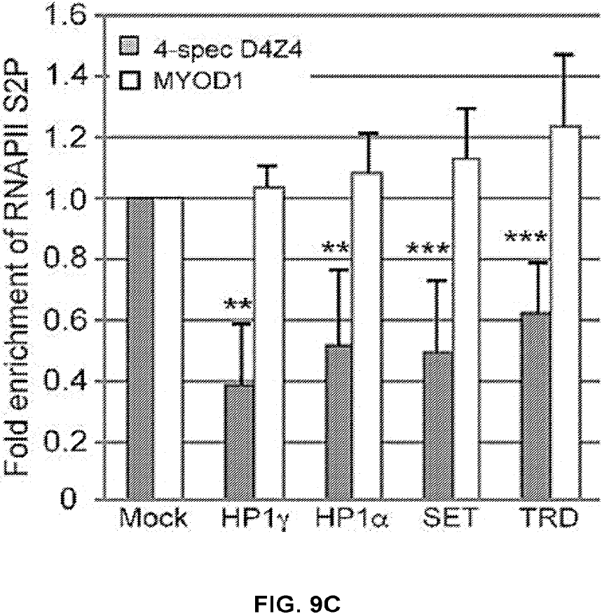

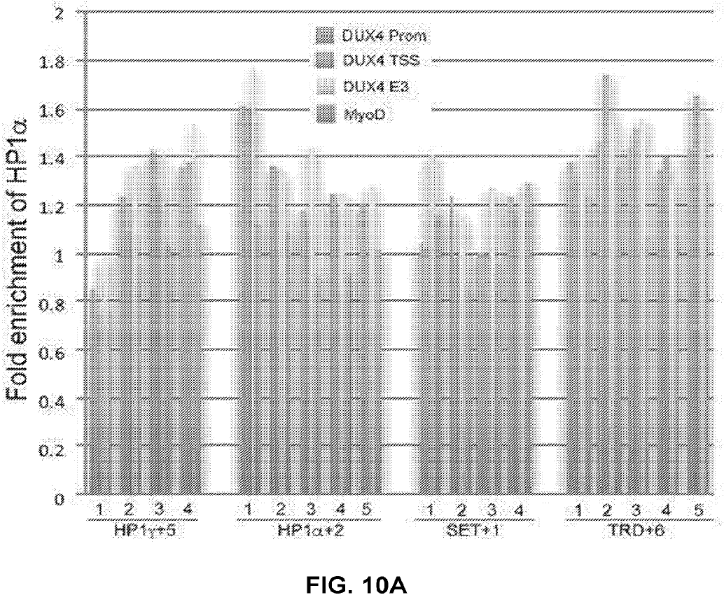

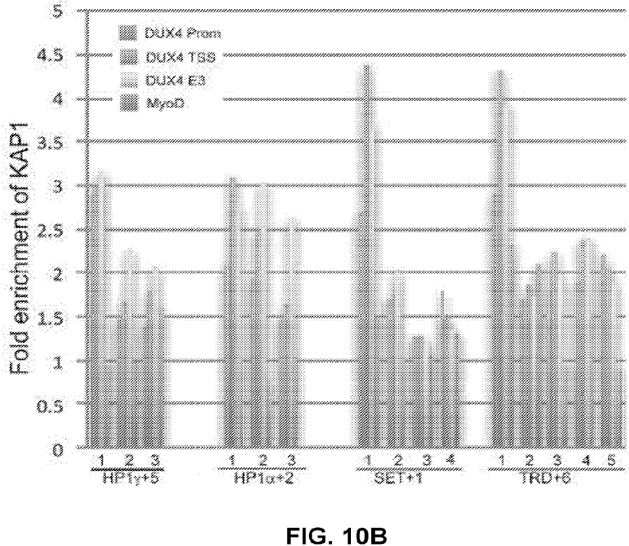

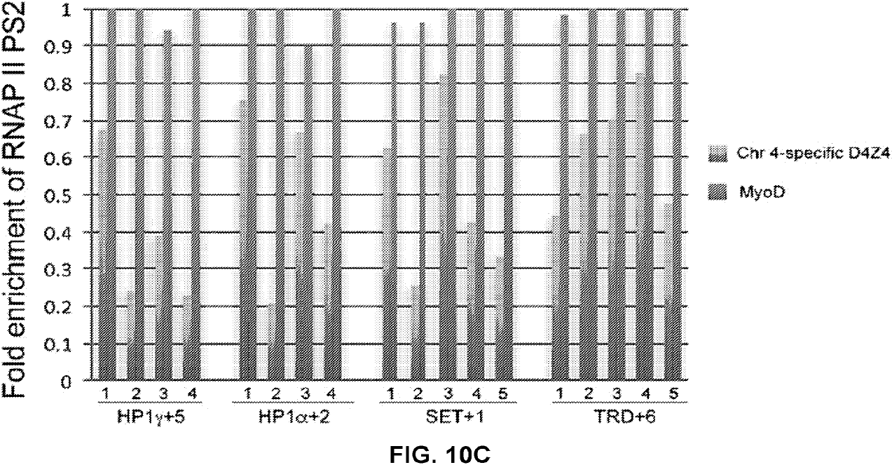

Example 3: dSaCas9-mediated recruitment of epigenetic repressors to DUX4 increases chromatin repression at the locus. Since targeting each epigenetic repressor to the DUX4 promoter/exon 1 reduced levels of DUX4-fl, it was expected that each would mediate direct changes in chromatin structure at the locus. Thus, ChIP assays were used to assess several marks of repressive chromatin across D4Z4 following CRISPRi treatment in FSHD myocytes. Increases in repressive marks are difficult to assess across the DUX4 locus, due to the fact that three of the four 4 q/10 q alleles are already in a compacted, heterochromatic state. Thus, any attempt to assess an increase in repression at the contracted allele is dampened by the presence of the other three. Unsurprisingly, changes in overall levels of the repressive H3K9me3 histone mark were undetectable across the D4Z4 repeats; however, other repressive marks were detectably and significantly elevated, overcoming the high background. Recruitment of HP1α to DUX4 led to a ~30-40% enrichment of this factor across the locus (FIGS. 9A and 10A), as well as increased occupancy of the KAP1 co-repressor (FIGS. 9B and 10B). Recruitment of HP1γ led to an increase in both HP1α and KAP1 at DUX4 exon 3, and recruitment of the MeCP2 TRD led to an increase in HP1α across the locus (FIGS. 9 and 10). Recruitment of each of the four factors also led to a ~40-60% decrease in the elongating form of RNA Pol II (phospho-serine 2) at the pathogenic repeat (FIGS. 9C and 10C), consistent with the lower levels of DUX4-fl mRNA observed (FIG. 2). Taken together, these results indicate that treatment with dSaCas9-repressors returns the chromatin at the disease locus to a more normal state of repression.

### Example 4: The FSHD-optimized Regulatory Cassette is only Active in Skeletal Muscles

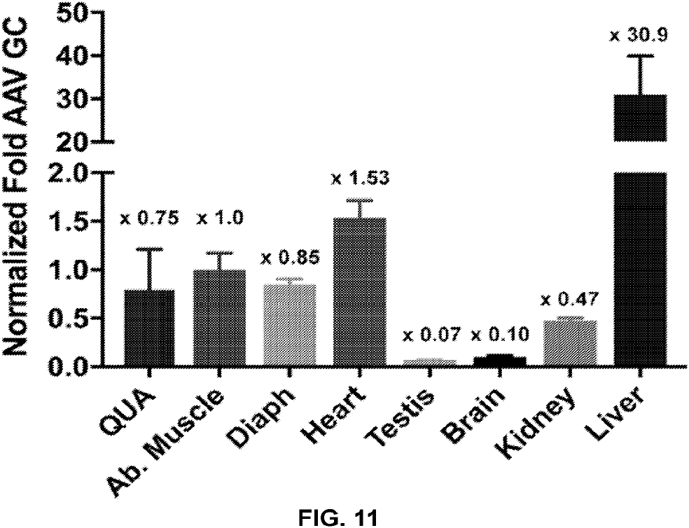

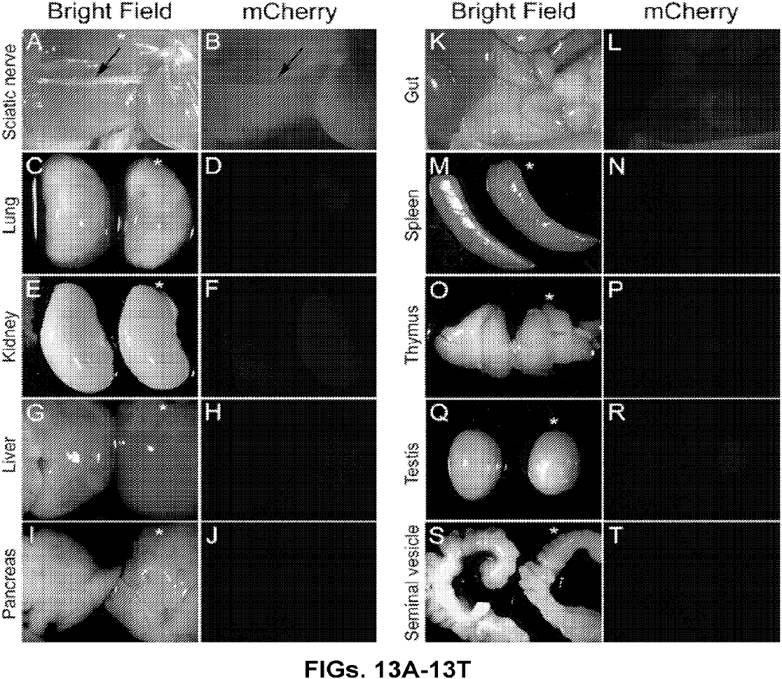

Following development of the optimized cassette, it was important to confirm that the smaller CKM-based regulatory cassette retains high activity in skeletal muscles with low to no activity in other tissues. Thus, in vivo expression of the FSHD-optimized regulatory cassette was analyzed using AAV9-mediated transgene delivery to wild-type mice. Viral particles were delivered by systemic retro-orbital injection (2.8x10¹⁴ genome copy [GC]/kg body weight), and the mCherry reporter signal was visualized at 12 wk post-injection. As previously reported for AAV9 vectors (Inagaki, et al. (2006) Mol Ther. 14:45-53), this vector strongly transduced skeletal muscles, cardiac muscle, and liver (FIG. 11). Nonetheless, mCherry expression was detected only in skeletal muscles and was undetectable in the heart (FIG. 12) and non-muscle tissues (FIG. 13), indicating that the FSHD-optimized regulatory cassette is only active in the critical target tissue. The lack of tropism/activity in testis is particularly important, since DUX4 is normally expressed in this tissue in healthy individuals.

### Example 5: Targeting dSaCas9-Repressors to DUX4 has Minimal Effects on the Muscle Transcriptome

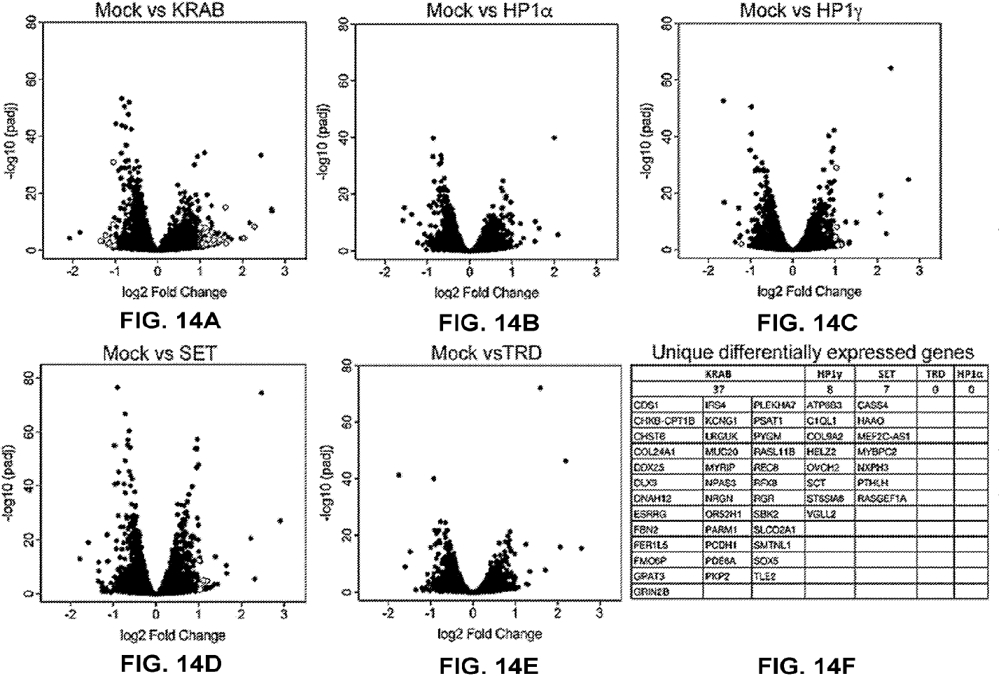

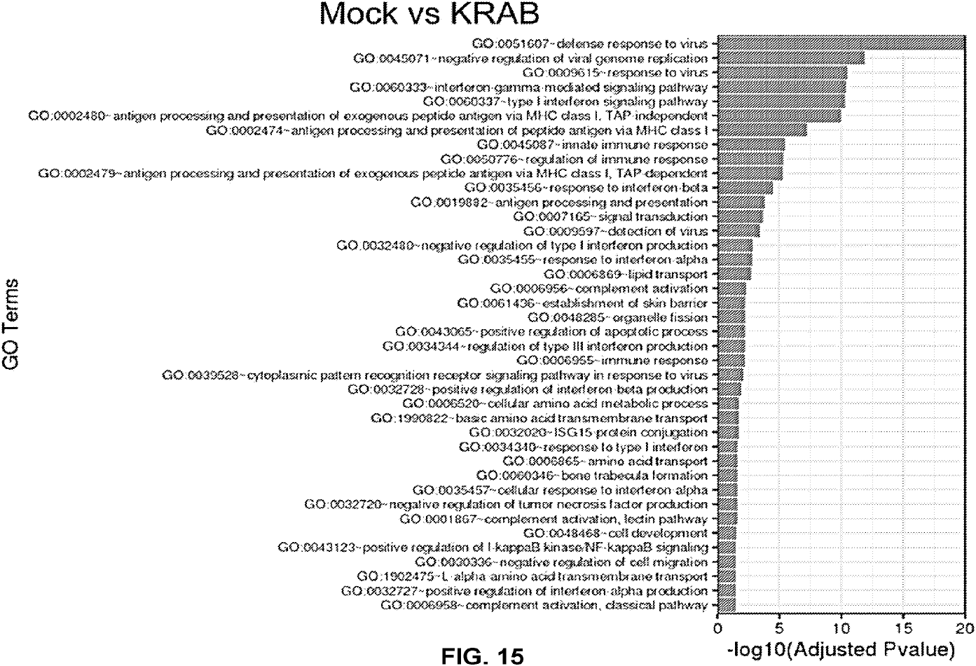

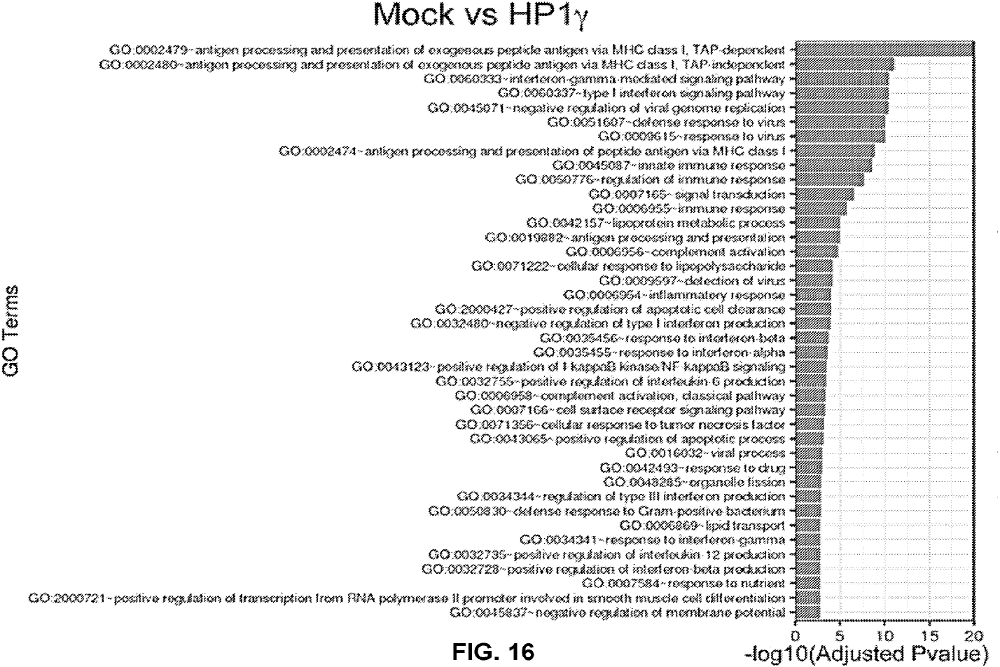

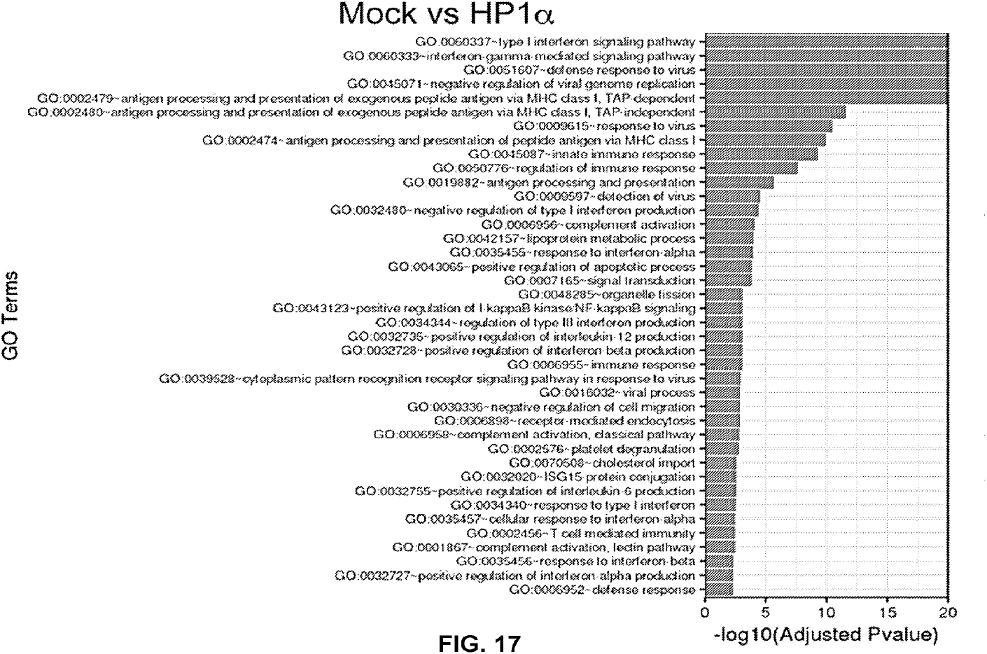

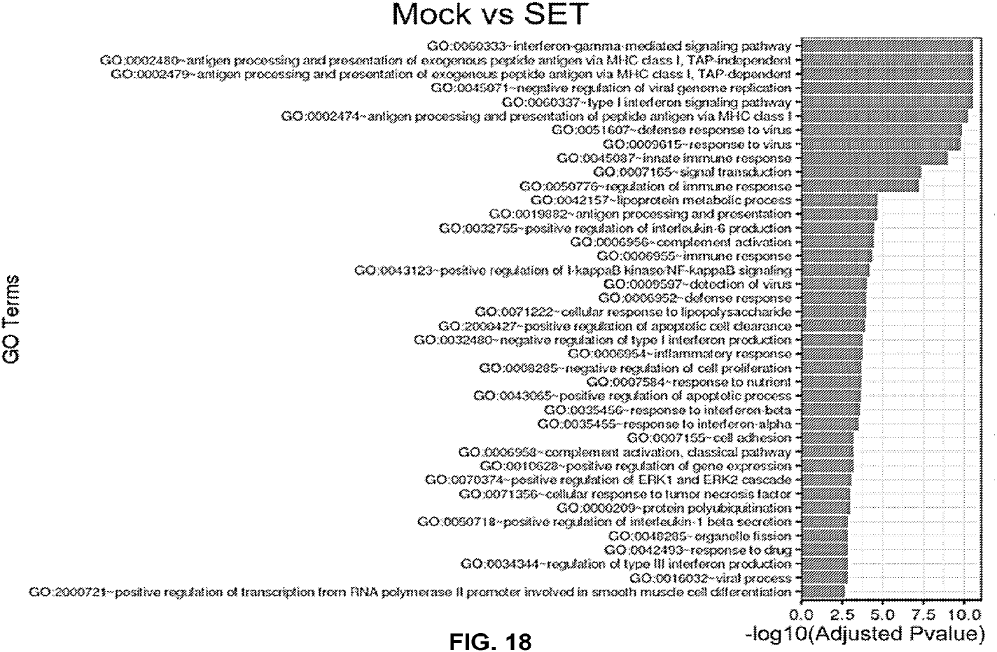

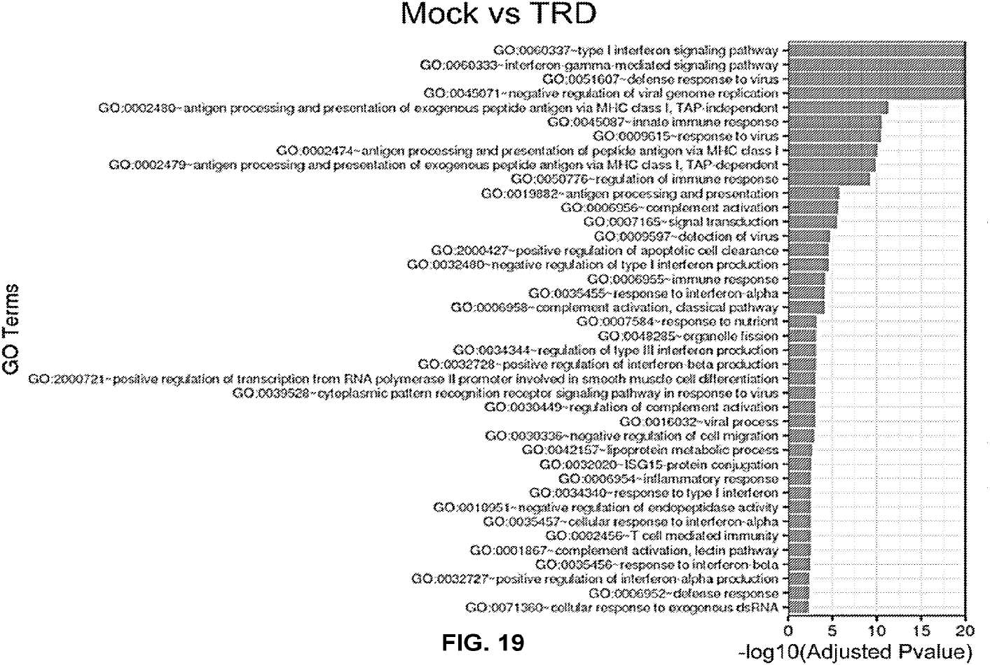

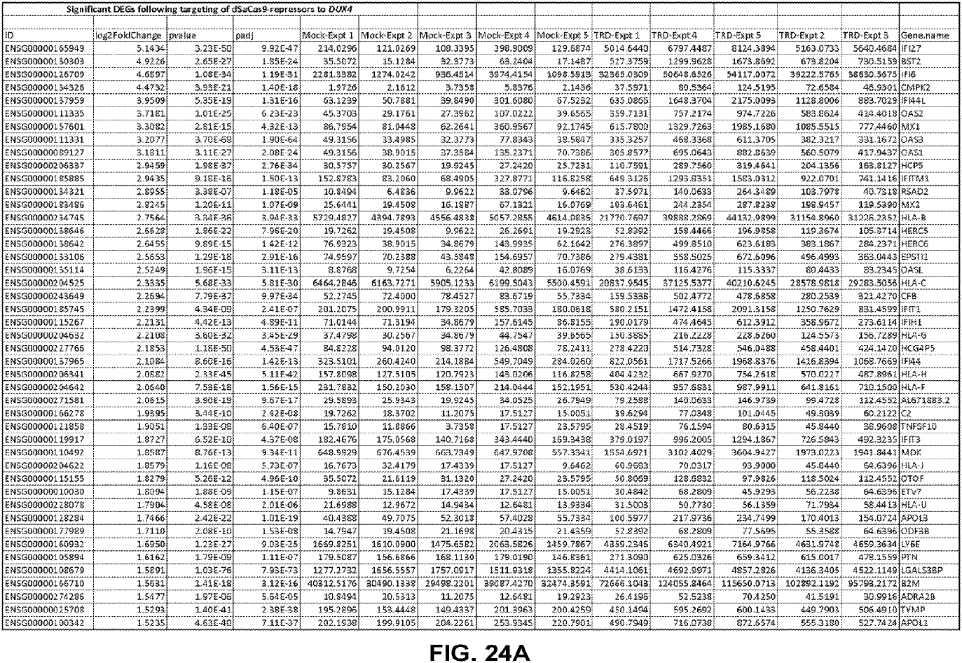

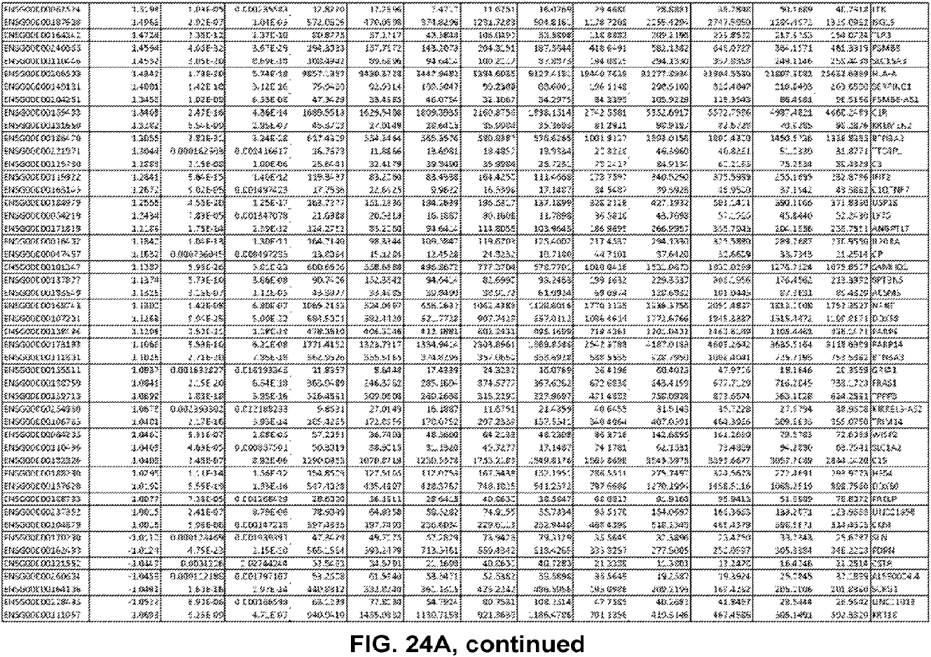

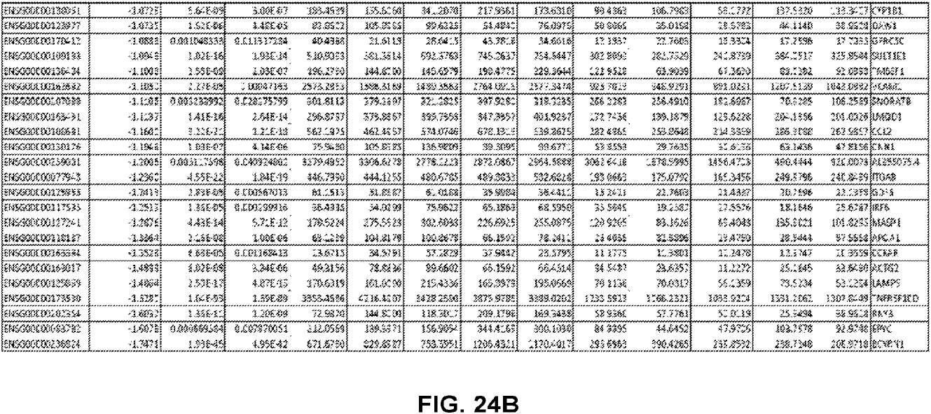

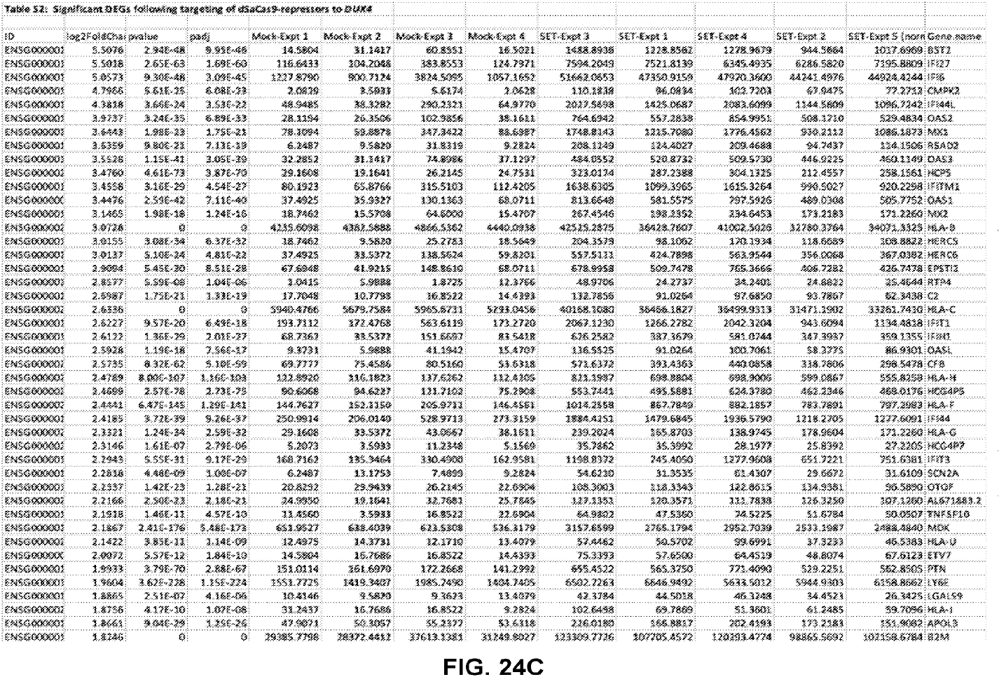

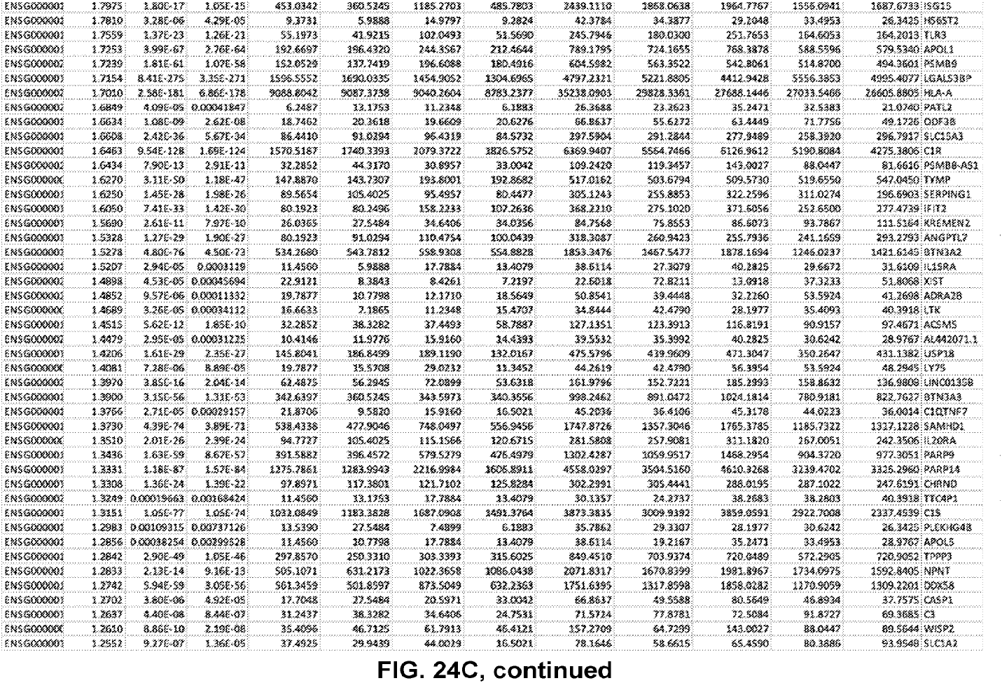

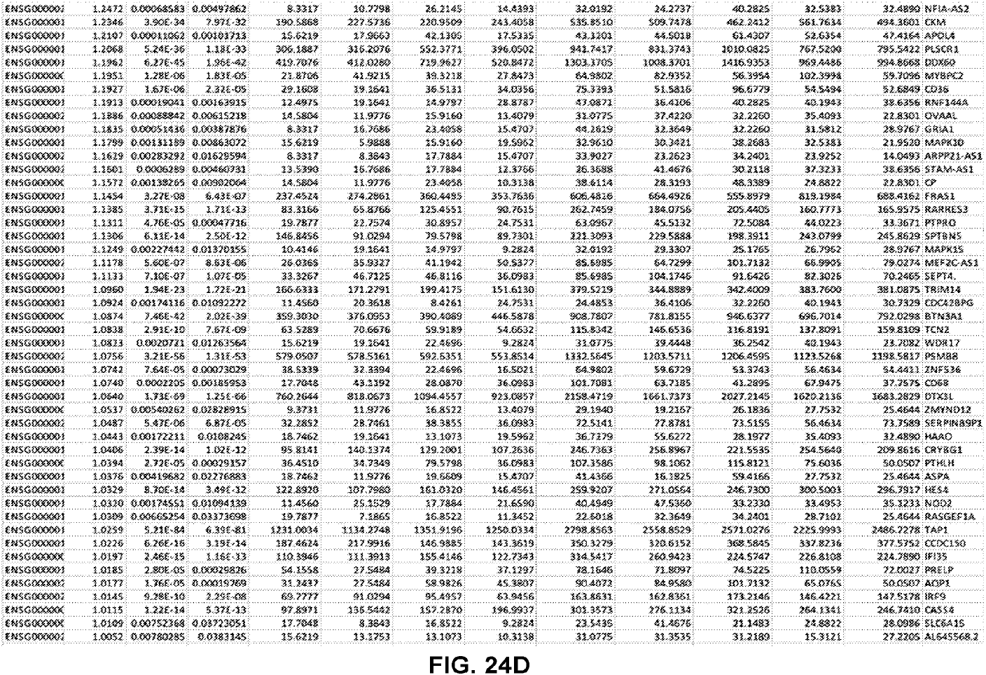

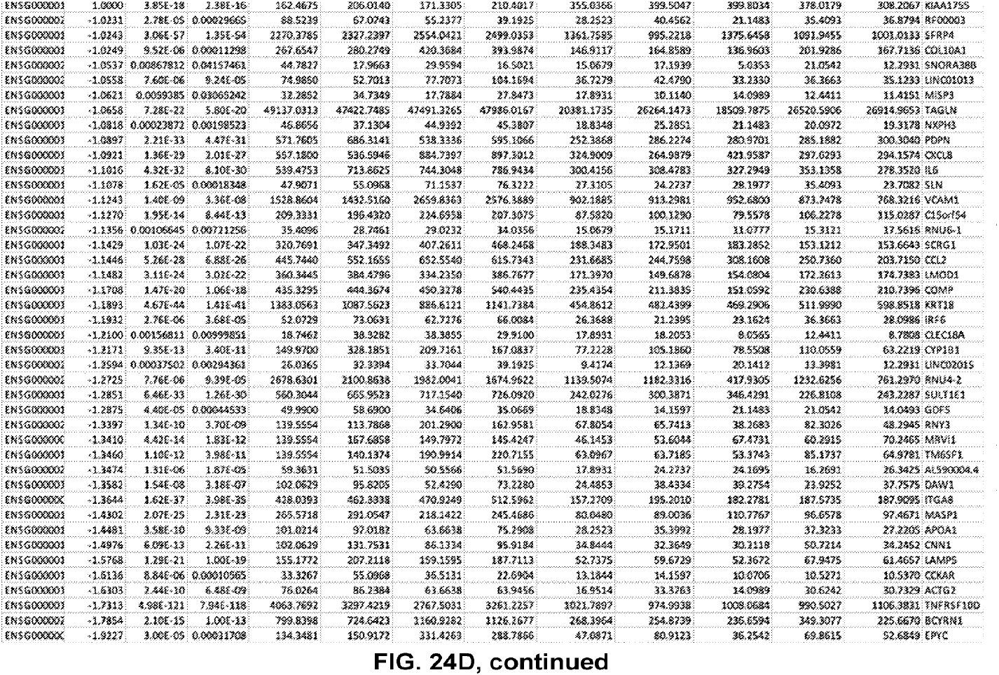

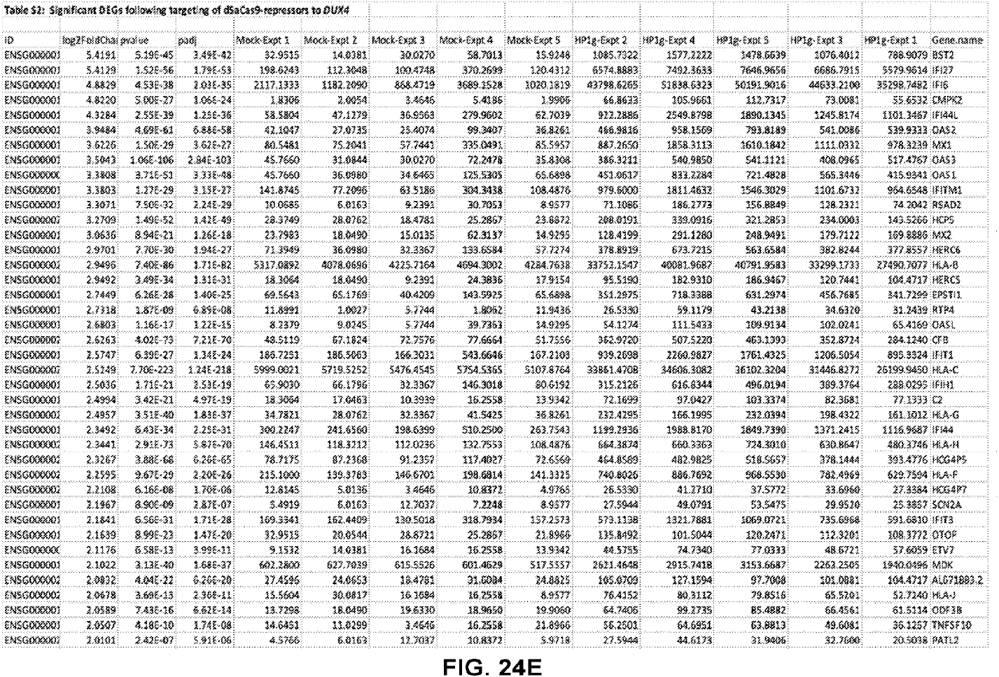

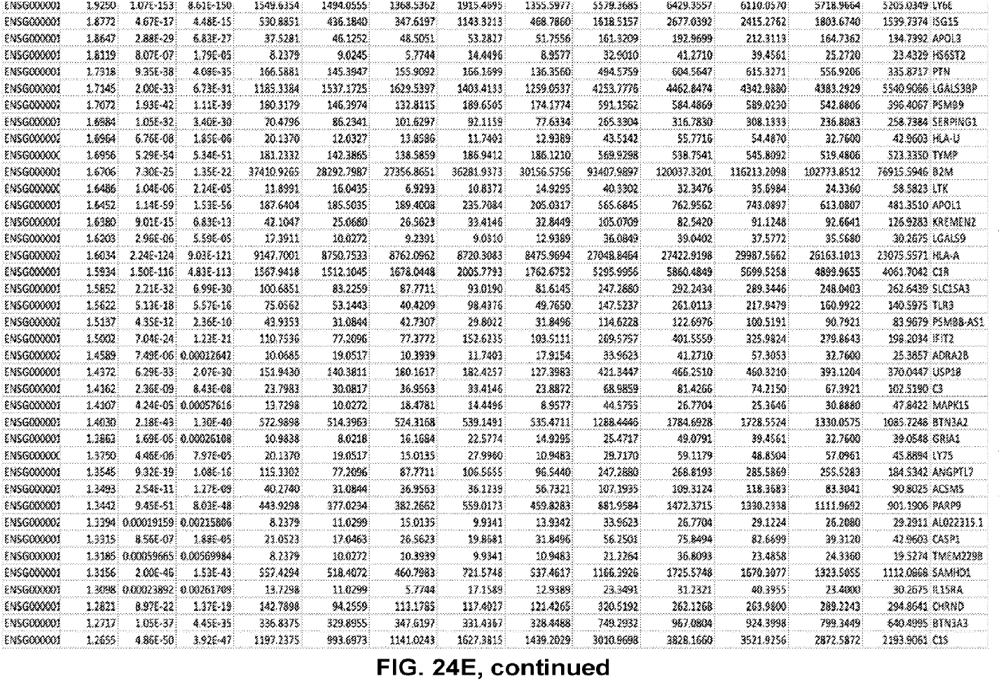

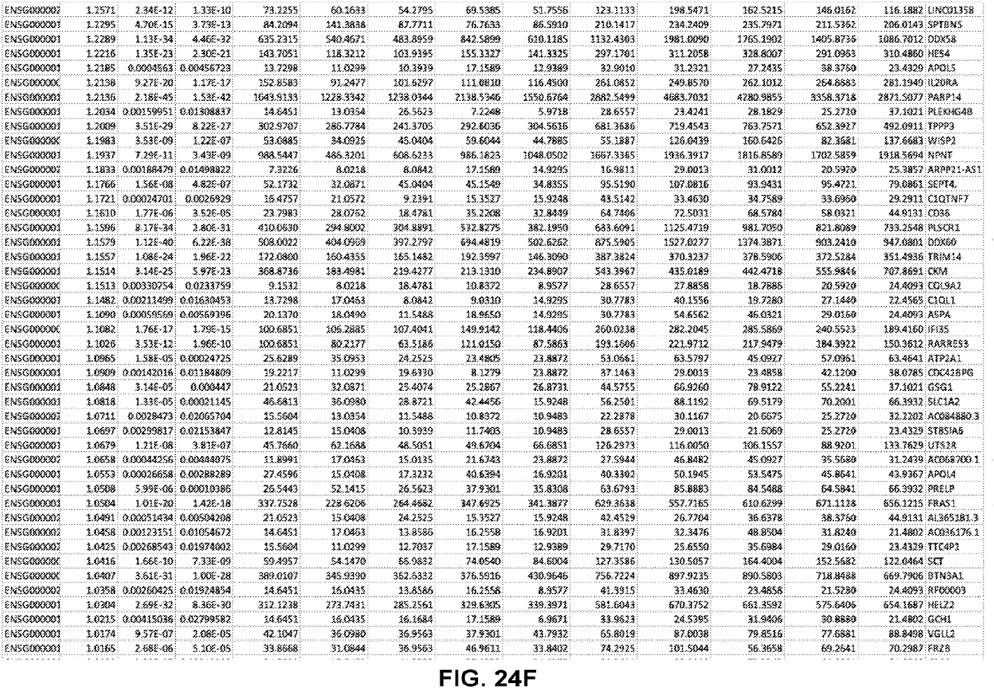

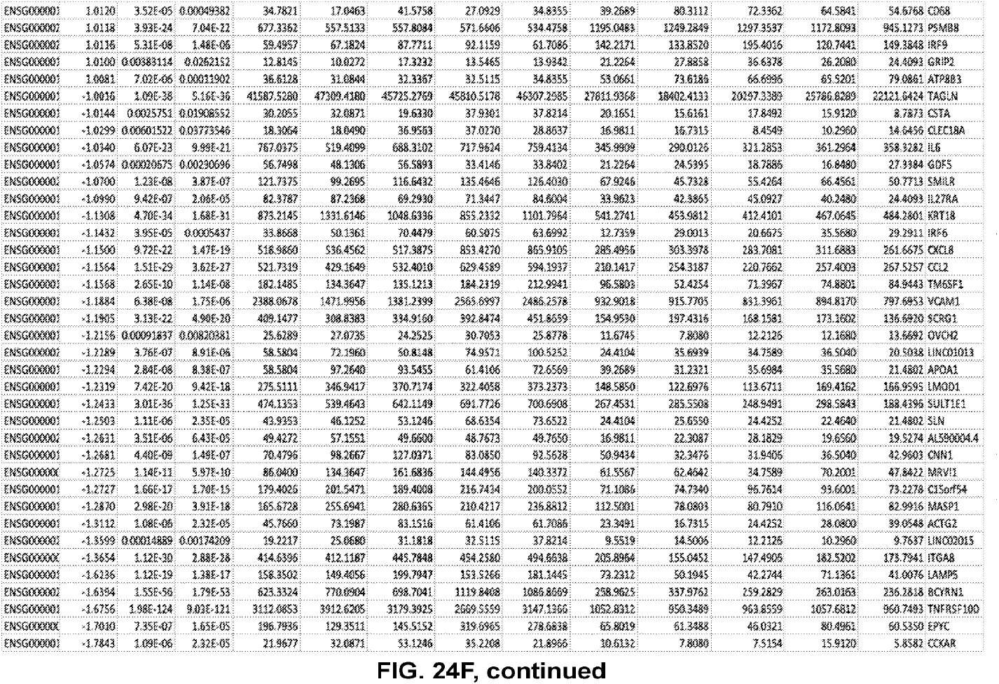

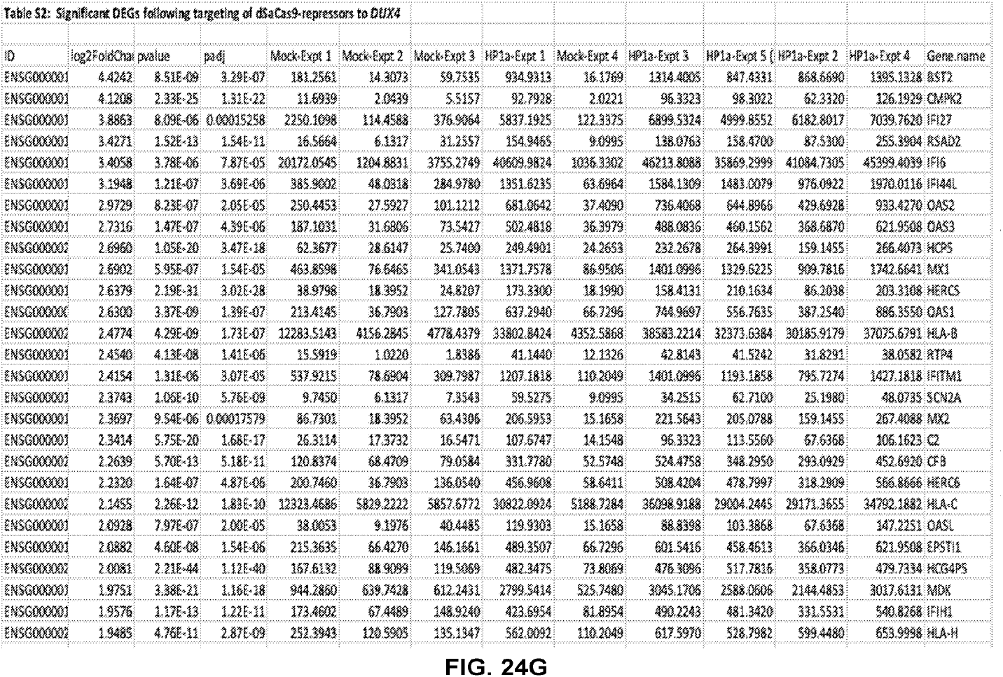

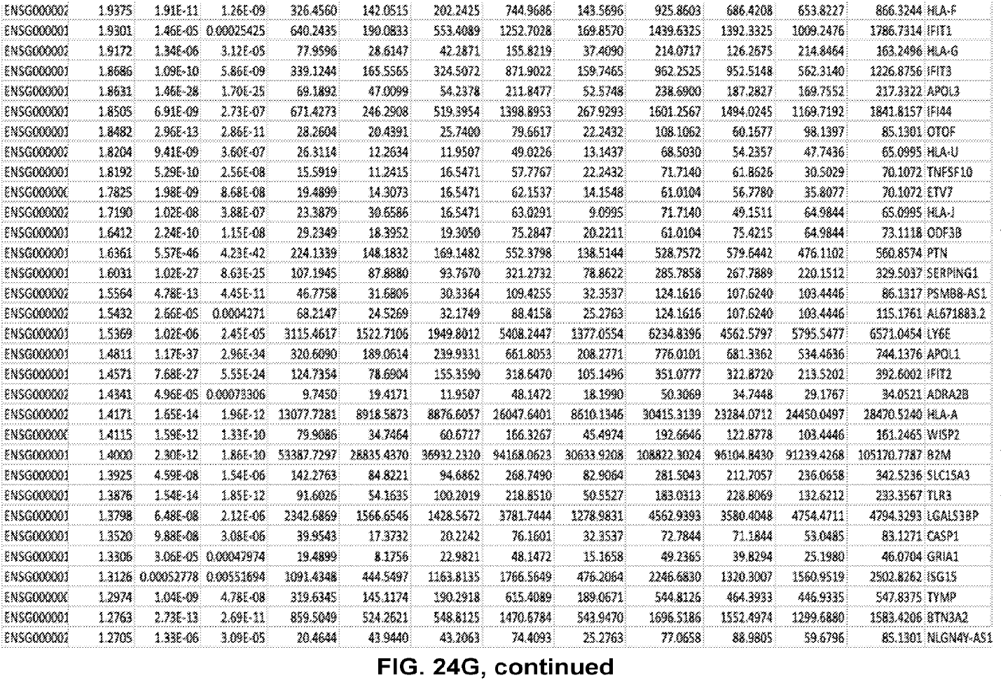

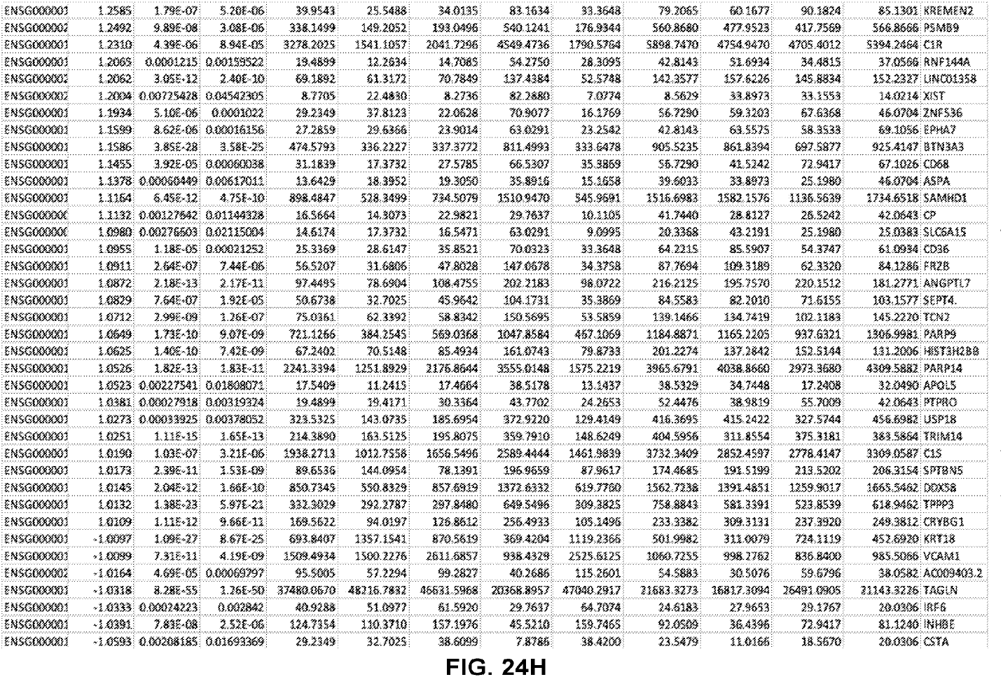

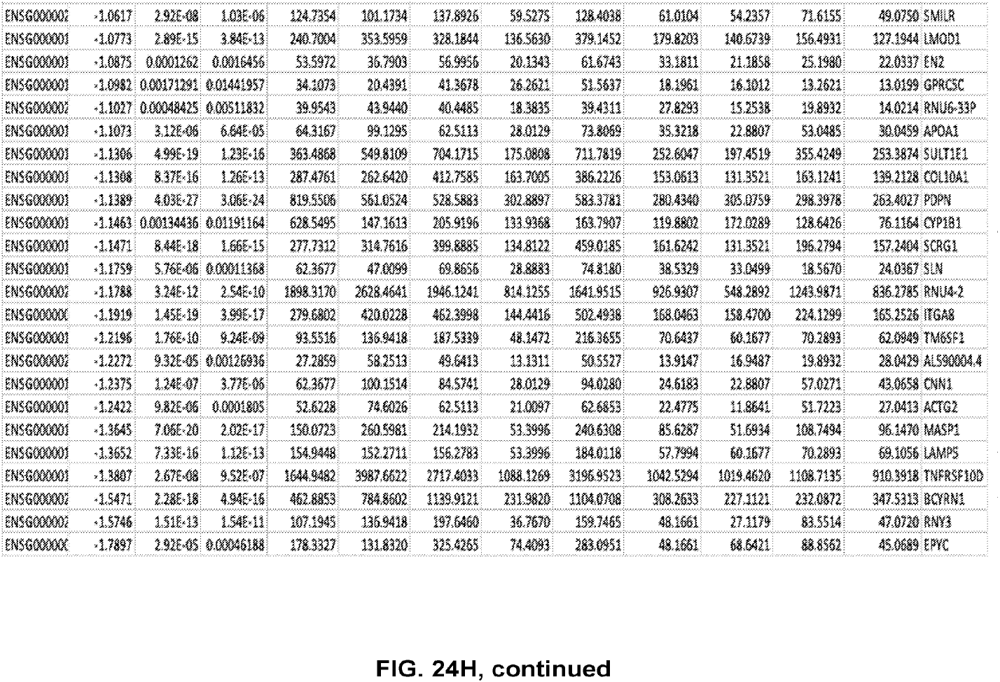

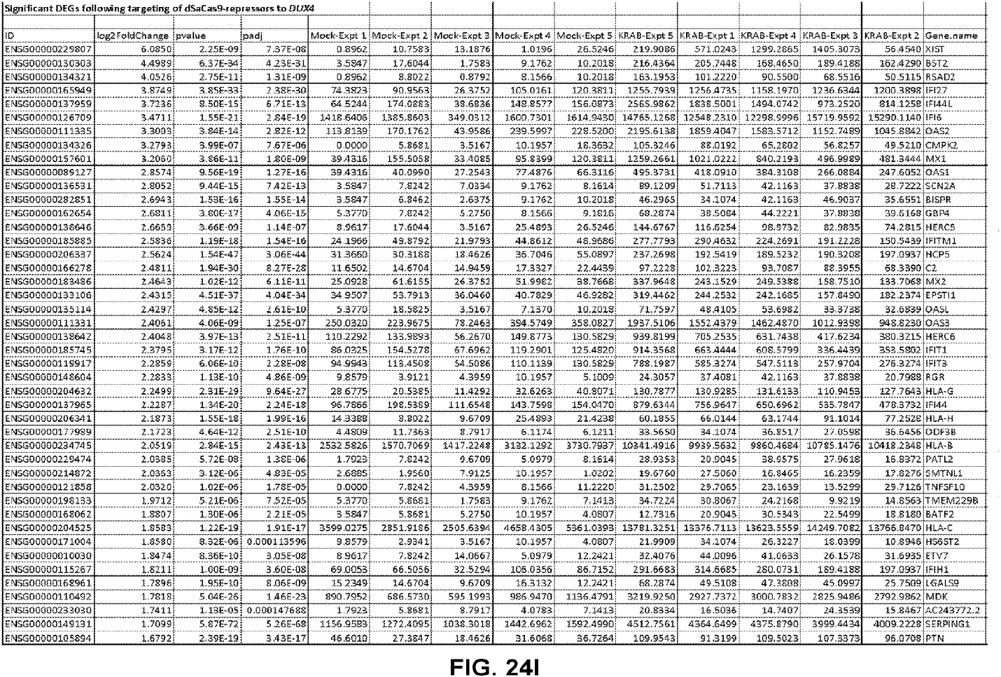

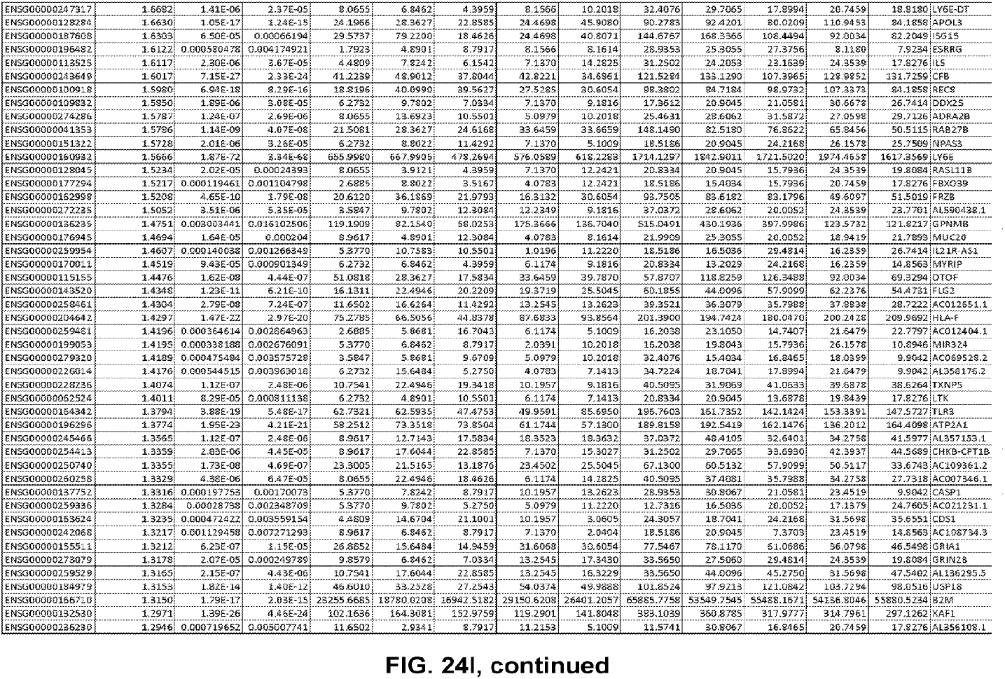

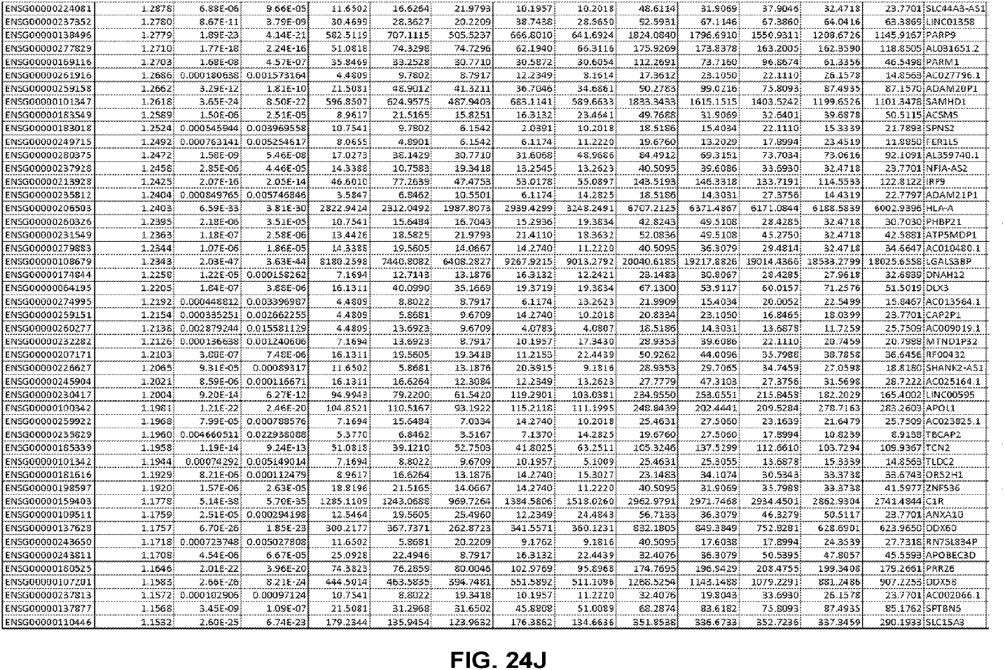

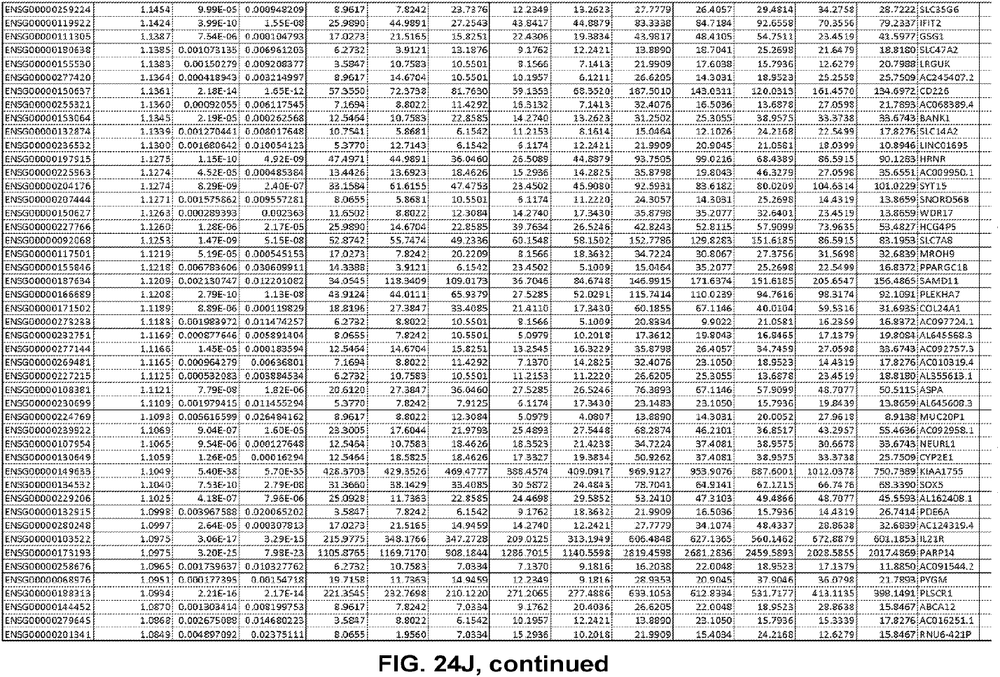

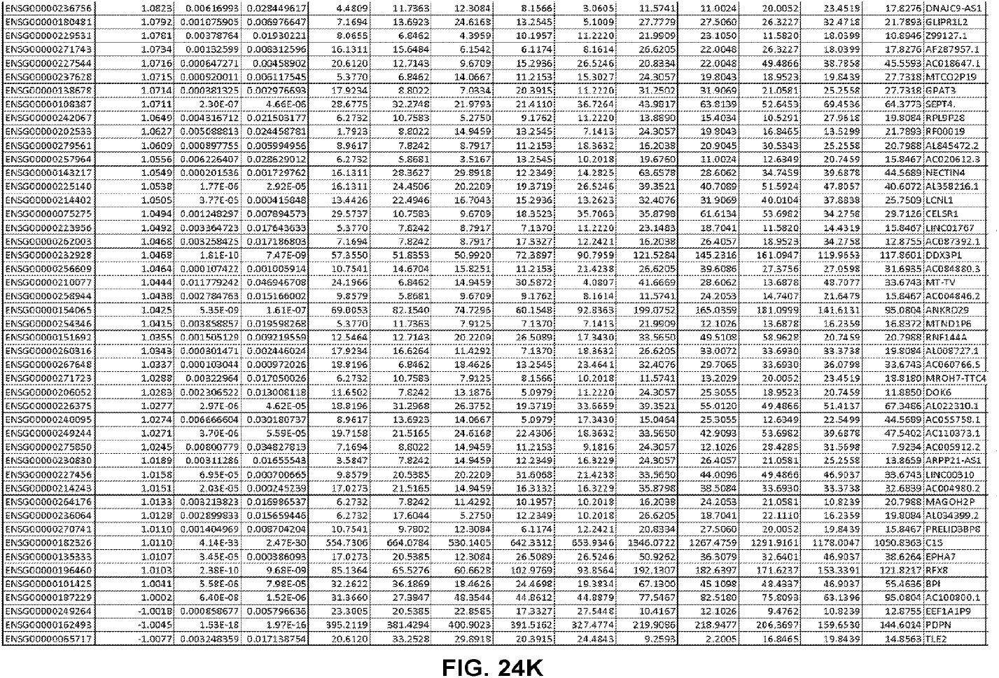

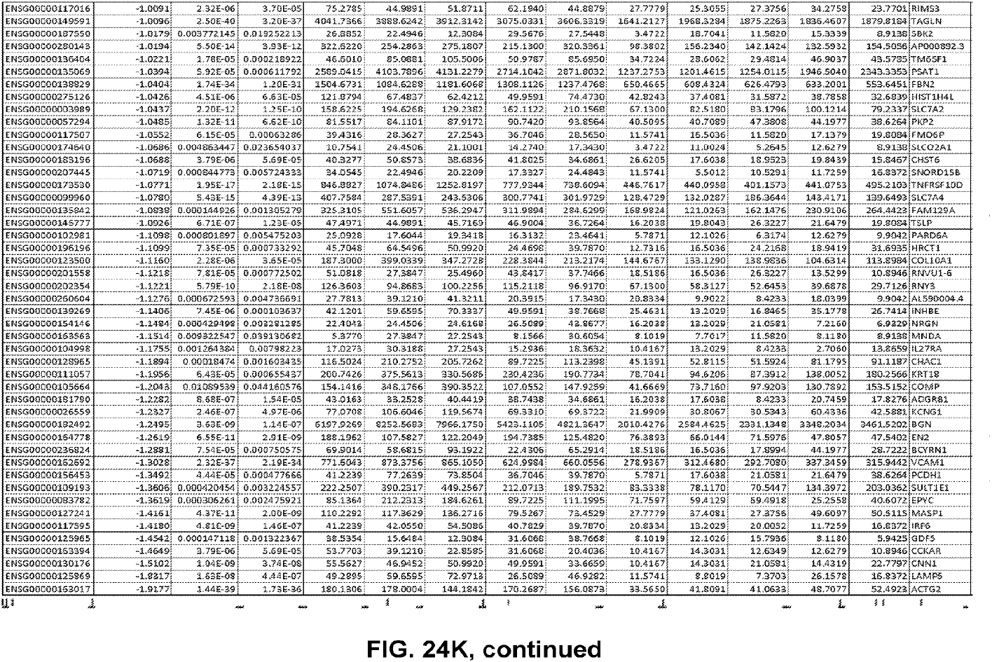

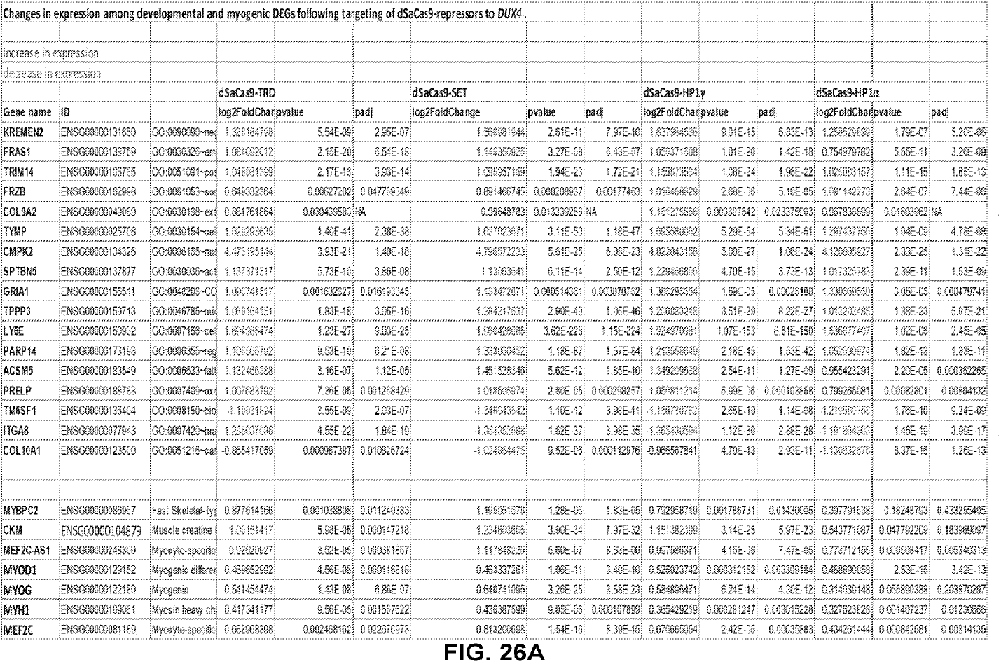

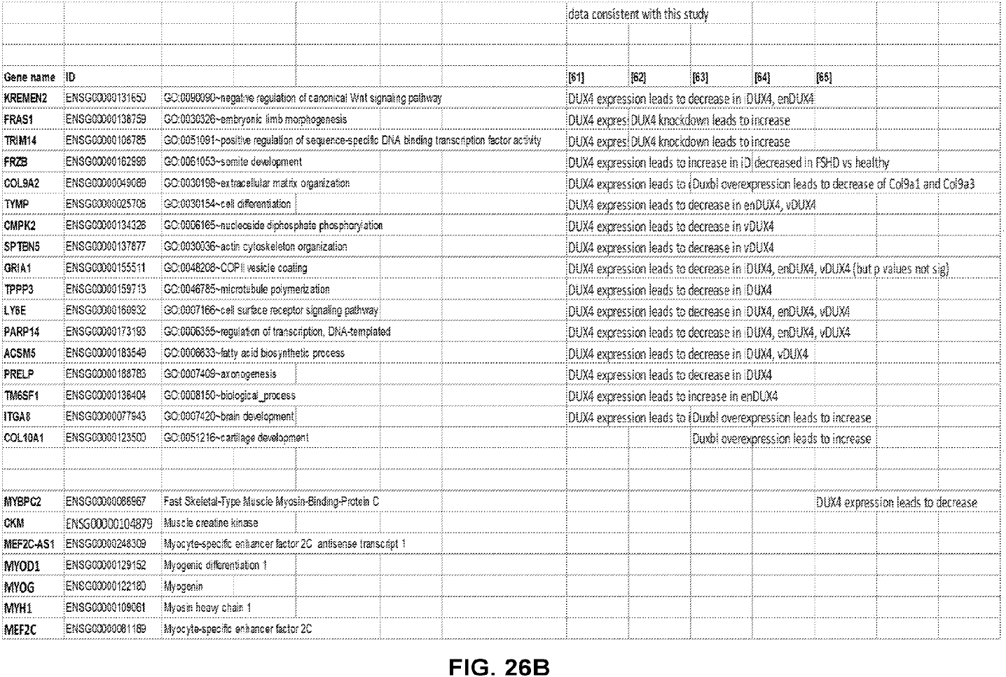

Since an analysis of off-target DNA binding (by ChIP-seq) sheds no light on the more critical off-target gene expression profiles, RNA-seq was performed to assess the global effects of targeting each dSaCas9-repressor to DUX4 with the most effective sgRNAs. Primary FSHD myocytes were transduced with each combination of vectors (described in FIG. 14) or with dSaCas9-KRAB + sgRNA #6 for comparison. Gene ontology (GO) analysis shows that the majority of misregulated cellular responses are likely due to the LV transduction or possibly dCas9 expression, and not due to off-target repression mediated by the dCas9-effectors (FIGS. 15-19). The fact that targeting with four different sgRNAs yields very similar profiles of differentially expressed genes (DEGs), consistent with an innate immune response, strongly supports this conclusion (FIGS. 24-26), although some immune-related DEGs may represent a correction of DUX4-mediated dysregulation, since DUX4 targets include immune mediators. After removing DEGs consistent with a response to virus, the vast majority remaining are part of embryonic programs or developmental pathways that are deregulated by DUX4 misexpression (FIG. 26 and Table 5). Many of these genes are common to multiple treatments and their differential expression represents a return to a more normal pattern of gene expression. For example, DUX4 expression reduces levels of TRIM14, KREMEN2, LY6E, and PARP14 in multiple independent studies (Jagannathan, et al. (2016) Hum. Mol. Genet. 25:4419-4431); consistent with these studies, all four dSaCas9-epigenetic repressor treatments led to an increase in expression of these genes. Conversely, TM6SF1 and ITGA8, which are upregulated following DUX4 overexpression (Jagannathan, et al. (2016) Hum. Mol. Genet. 25:4419-4431), were both decreased following treatment with every dSaCas9-epigenetic repressor.

Expression levels of myogenic genes that were assessed by qRT-PCR (MYOD1, MYOG, and MYH1) also showed no changes by RNA-seq analysis (FIG. 26). The only muscle genes with differential expression are CKM, which is increased ~2-fold following treatment with dSaCas9-SET, -HP1γ, or -TRD, an antisense transcript of MEF2C, and MYBPC2, which are increased ~2-fold following treatment with dSaCas9-SET (FIG. 26). Since DUX4 expression is reported to inhibit myogenesis, these changes also likely represent a beneficial correction of DUX4-mediated transcriptional dysregulation.

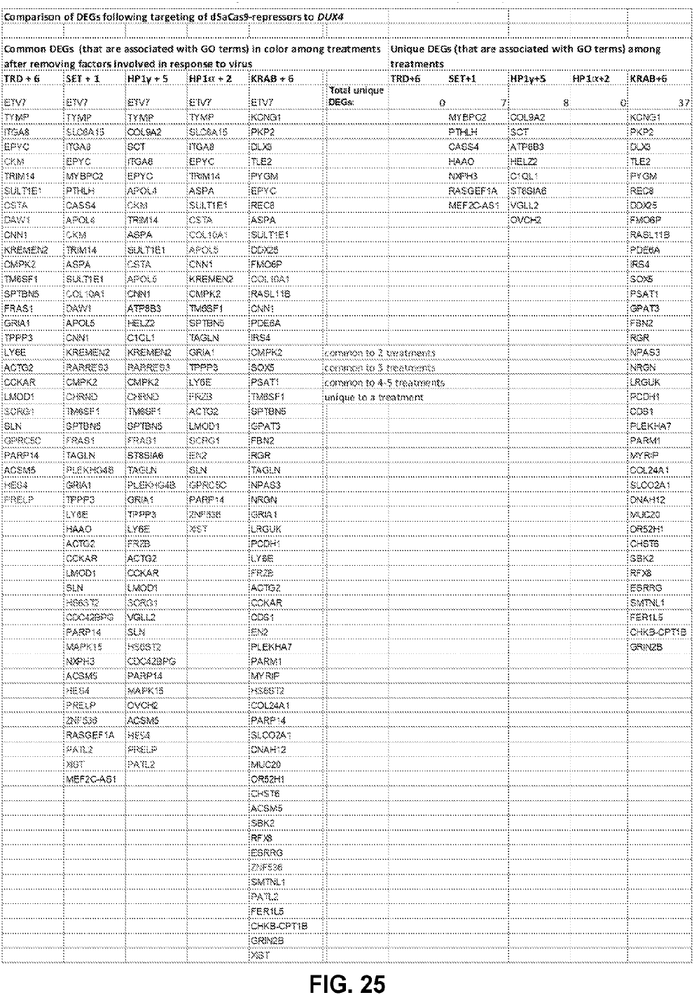

Importantly, the number of detectable off-target responses to each treatment was extremely low. Treatment with dSaCas9-TRD or -HP1α yielded no significant unique DEGs, while treatment with dSaCas9-SET and -HP1γ yielded only 7 and 8 unique DEGs, respectively (FIG. 14, FIG. 24, and FIG. 25). Thus, as predicted by the in silico search of sgRNA targets, this system for CRISPRi is highly specific in human myocytes. In contrast, treatment with dSaCas9-KRAB, which was targeted using the same sgRNA (#6) as dSaCas9-TRD, yielded 37 unique DEGs (vs. 0 for dSaCas9-TRD). This result was surprising, considering the high specificity reported for dSpCas9-KRAB; however, it suggests that, without wishing to be bound by theory, at least in myocytes, the KRAB repressor is recruited to genomic locations independent of sgRNA targeting and is a more promiscuous repressor than the MeCP2 TRD.

Table 5. Changes in DUX4-dependent gene expression following targeting of dSaCas9-repressors to DUX4. Shown are log2fold changes in DUX4 target genes whose expression in FSHD myocytes is altered following transduction with each dSaCas9-repressor + DUX4-targeting sgRNA. These genes are part of developmental pathways dysregulated by DUX4, and their differential expression following CRISPRi treatment represents a return to a more normal pattern of gene expression. (NS, not significant).

| | dSaCas9-TRD | dSaCas9-SET | dSaCas9-HP1γ | dSaCas9-HP1α |
|---|---|---|---|---|
| KREMEN2 | 1.33 | 1.57 | 1.64 | 1.26 |
| FRAS1 | 1.08 | 1.15 | 1.05 | NS |
| TRIM14 | 1.05 | 1.10 | 1.16 | 1.03 |
| FRZB | NS | NS | 1.02 | 1.09 |
| COL9A2 | NS | NS | 1.15 | NS |
| TYMP | 1.53 | 1.63 | 1.70 | 1.30 |
| CMPK2 | 4.47 | 4.80 | 4.82 | 4.12 |
| SPTBN5 | 1.14 | 1.13 | 1.23 | 1.02 |
| GRIA1 | 1.09 | 1.18 | 1.39 | 1.33 |
| TPPP3 | 1.07 | 1.28 | 1.20 | 1.01 |
| LY6E | 1.69 | 1.96 | 1.92 | 1.54 |
| PARP14 | 1.11 | 1.33 | 1.21 | 1.05 |
| ACSM5 | 1.13 | 1.45 | 1.35 | NS |
| PRELP | 1.01 | 1.02 | 1.05 | NS |
| TM6SF1 | -1.10 | -1.35 | -1.16 | -1.22 |
| ITGA8 | -1.24 | -1.36 | -1.37 | -1.19 |
| COL10A1 | NS | -1.02 | NS | -1.13 |

### Example 6: In Vivo Targeting of dSaCas9-Repressors to DUX4 Exon 1 Represses DUX4-fl and DUX4-FL Targets in ACTA1-MCM; FLExDUX4 Bi-Transgenic Mice

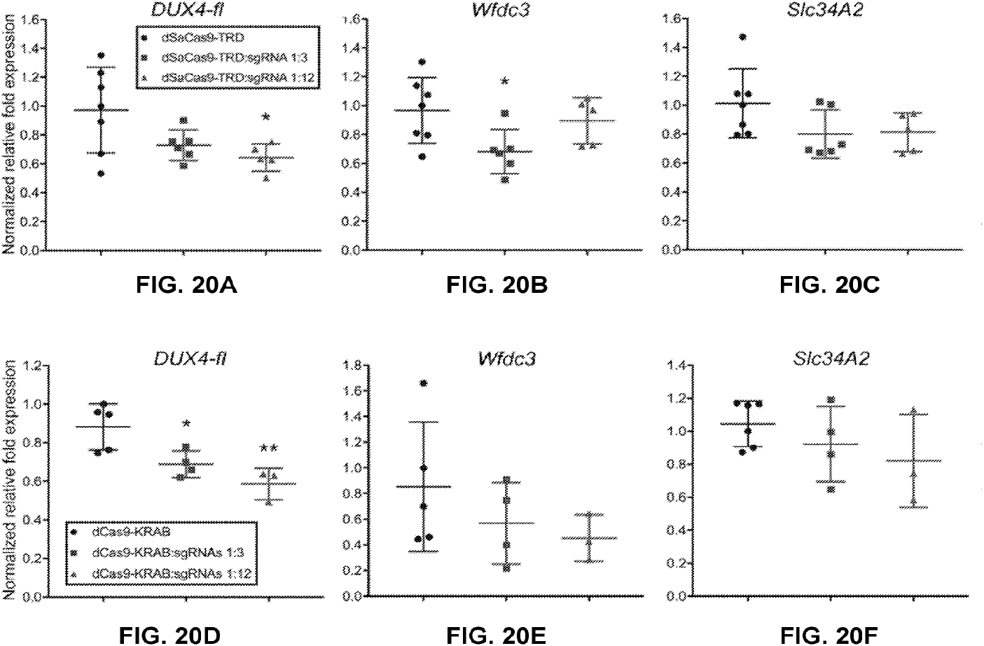

To test the ability of the CRISPRi platform to repress DUX4-fl in vivo, the ACTA1-MCM; FLExDUX4 (FLExD) FSHD-like bi-transgenic mouse model was utilized, which can be induced to express DUX4-fl and develop a moderate pathology in response to a low dose of tamoxifen (Jones, et al. (2020) Skelet. Muscle 10,8). These mice carry one human D4Z4 repeat from which DUX4-fl is expressed and can be targeted by sgRNAs to exon 1. Mice were injected intramuscularly with AAV9 vectors encoding dSaCas9-TRD or -KRAB and sgRNAs targeting DUX4 exon 1 at different ratios, followed 3.5 weeks later by intraperitoneal injection of tamoxifen to induce mosaic DUX4-fl expression in skeletal muscles. Two weeks post-induction, expression of DUX4-fl and the mouse homologs of two direct target genes that are robustly induced by DUX4-FL were assessed by qRT-PCR in the injected TAs. Although transcript levels of the DUX4-fl transgene are difficult to assess in this model, targeting either dCas9-TRD or -KRAB to DUX4 exon 1 led to a -30% decrease in expression of DUX4-fl at the higher ratio of sgRNA to effector (FIG. 20). Transcript levels of DUX4-FL targets Wfdc3 and Slc34a2 were also reduced, although the reduction was only significant for dCas9-TRD at the lower ratio of sgRNA to effector (FIG. 20). Although these effects are modest, they provide proof-of-principle that this epigenetic CRISPRi platform is a viable strategy for ongoing preclinical development.

Example 7: Design of CRISPRi all-in-one vectors and validation in cultured primary FSHD myocytes. Following the successful proof-of-principle (Himeda, et al. (2020) Mol Ther Methods Clin Dev. 20:298-311), the therapeutic cassettes were re-engineered to accommodate all CRISPRi components (dSaCas9 fused to each epigenetic regulator and its targeting sgRNA) within single vectors (FIG. 1C). This is critical for bringing CRISPRi to the clinic, as it eliminates the need for two viruses, thus: 1) increasing the efficiency of delivery, 2) reducing the high cost of therapy, and 3) reducing the immunotoxicity associated with high viral doses. Four all-in-one CRISPRi therapeutic cassettes were initially engineered in lentiviral (LV) vectors. Importantly, size of the cassettes was restricted to <4.4 kb total so that each would be amenable for use in AAV. Accommodating all CRISPRi components within this size constraint required further minimization of the therapeutic cassettes; thus, HP1α and HPly were trimmed to their essential chromo shadow and C-terminal extension domains, and pre- and post-SET domains were eliminated from the SUV39H1 cassette. Each all-in-one vector contains: 1) dSaCas9 fused to one of five repressors (either the HP1α or HPly chromo shadow domain and C-terminal extension, the MeCP2 TRD, or the SUV39H1 SET domain) under control of the FSHD-optimized regulatory cassette, and 2) an sgRNA targeting the DUX4 promoter/exon 1 under control of the U6 promoter (FIG. 1C, Table 3, and Table 4). Control vectors contain each dSaCas9-repressor in conjunction with a non-targeting sgRNA.

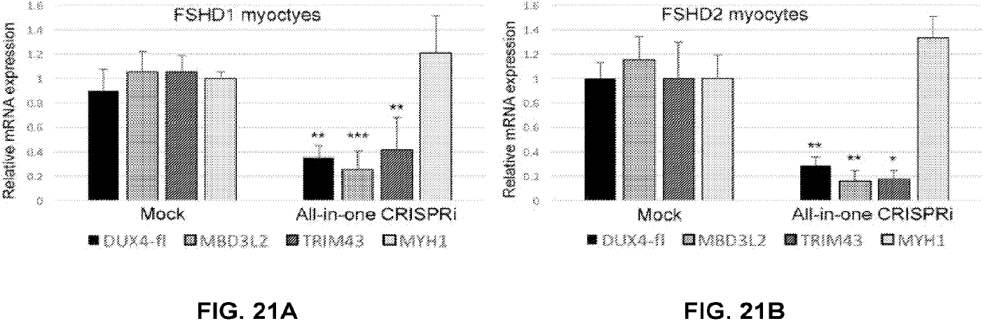

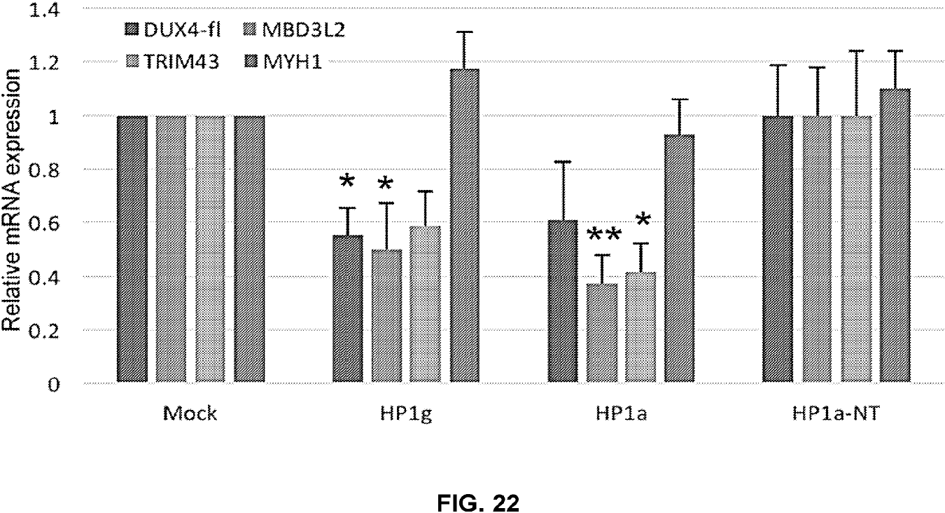

Using dSaCas9-TRD as proof-of-principle, this single-vector system for CRISPRi effectively represses DUX4 and its targets in both primary FSHD1 and FSHD2 myocytes (FIG. 21). Importantly, this is the first demonstration that CRISPRi targeting DUX4 can be effective in FSHD2 patient cells. In addition, trimming HP1α and HP1γ to their essential chromo shadow and C-terminal extension domains still allows for effective repression of DUX4-fl and its target genes in FSHD1 myocytes (FIG. 22).

### Example 8: The Modified FSHD-optimized Regulatory Cassette Displays Increased Activity in Soleus, Diaphragm, and Heart

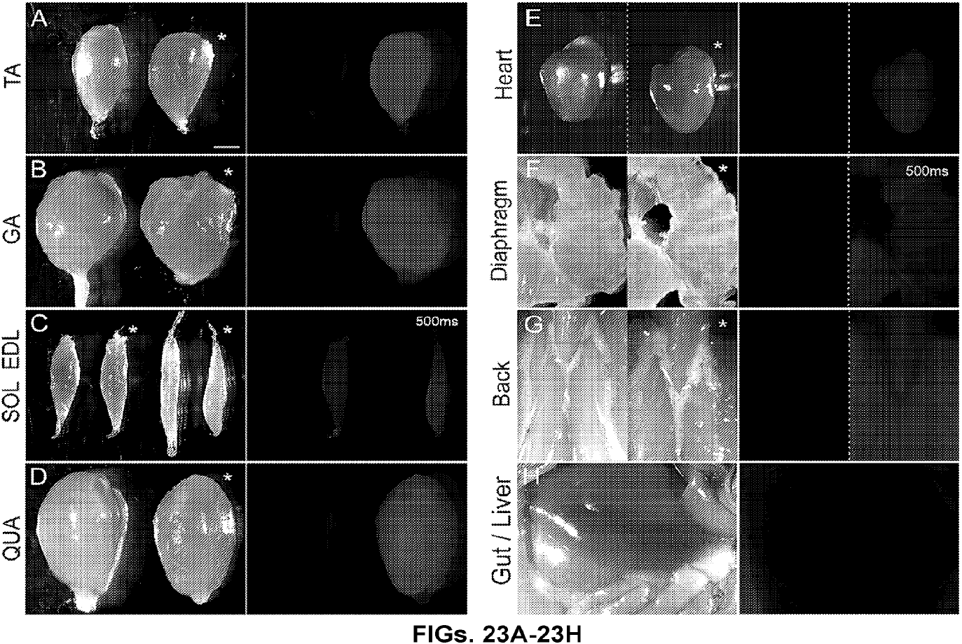

Although the current vector expresses very highly in fast-twitch muscles, one weakness is the lack of expression in soleus and diaphragm (Himeda, et al. (2020) Mol Ther Methods Clin Dev. 20:298-311). To address this, the regulatory cassette was redesigned to replace the additional Right E-box with the original Left E-box from the CKM enhancer (Himeda, et al. (2011) Methods Mol. Biol. 709:3-19). This modification increased cassette activity in both soleus and diaphragm, as well as in the heart. Importantly, the new cassette still displayed very high activity in fast-twitch muscles, with no detectable expression in non-muscle tissues (FIG. 23). While expression in the heart is not necessary for an FSHD-specific cassette, targeting a repressor to DUX4 in a tissue where the DUX4 locus is already repressed (such as cardiac muscle) shouldn’t cause any negative effects.

Example 9: Discussion. There are no cures or ameliorative treatments for FSHD, so an effective therapy is critically needed. Since the discovery that FSHD pathogenesis is caused by aberrant expression of DUX4 in skeletal muscles, numerous therapeutic approaches targeting DUX4 and its downstream pathways are being developed. While small molecules targeting DUX4 expression, independently identified from highly similar indirect expression screens, are promising, their discovery is limited by the chemical libraries screened, dosing, and modes of action. Despite the clear overlap in libraries, two published screens with similar approaches identified different molecules, targets, and pathways for DUX4 inhibition, even to the exclusion of other targets (Cruz, et al.(2018) J Biol Chem.; Campbell, et al.(2017) Skelet Muscle.7:16), which is cause for concern. Others have focused on targeting DUX4 activity or toxicity (Choi, et al. (2016) J Biomol Screen. 21:680-8; Bosnakovski, et al.(2014) Skelet Muscle. 4:4; Bosnakovski, et al.(2019) Sci Adv. 5:7781); however, these involve ubiquitous and robust cellular pathways and it is not clear which, if any, are causal for pathology. It is worth emphasizing that many treatments have been quite successful in preclinical studies only to fail during clinical trials. Recent events in the field of myotonic dystrophy have underscored the importance of not abandoning alternative avenues of potential therapy in the wake of a single promising treatment. In all reports targeting DUX4 expression or activity, global effects of inhibition have not been investigated, and most known targets are ubiquitous cellular effectors whose inhibition is likely to have significant undesired effects, particularly during the long-term dosing required for FSHD.

The most direct path to an FSHD therapy is eliminating expression of DUX4 mRNA. While the amount of DUX4 inhibition required for effective therapy is unknown, data from clinically affected and asymptomatic FSHD subjects support that any reduction in DUX4 expression will have therapeutic benefit (Jones, et al.(2012) Hum Mol Genet. 21:4419-30; Wang, et al.(2019) Hum Mol Genet. 28:476-486).. However, both DUX4 and the D4Z4 repeat that encodes it present unique therapeutic challenges. For example, although highly similar D4Z4 repeat arrays are found at multiple loci in the genome, DUX4 is only stably expressed from the distal-most repeat unit on a permissive allele. In addition, while other mammals contain functional orthologs, the D4Z4 array and intact DUX4 gene are not conserved outside of old-world primates and no natural animal models exist.

CRISPR/Cas9 technology has been used extensively to target and modify specific genomic regions, offering the potential for permanent correction of many diseases. While the dangers associated with standard CRISPR editing are a concern for any locus, they are of particular concern in a highly repetitive region such as the FSHD locus. However, the use of CRISPR to repress gene expression is ideally suited to FSHD. Unfortunately, CRISPRi platforms for human gene therapy are limited by the large size of Cas9 targeting proteins, which take up most of the available space in AAV vectors, leaving little room for effectors. Not surprisingly, most proof-of-principle studies have utilized dSpCas9 in LV vectors, which have a larger genome capacity and are convenient for expression in cultured cells, but not useful for clinical gene delivery. The smaller dSaCas9 ortholog has been shown to work well with a fused effector (Josipovic, et al.(2019) J Biotechnol. 301: 18-23), but its coding sequence is still over 3 kb, leaving little room for a chromatin modulator and regulatory sequences within the 4.4 kb packaging capacity of AAV. It is worth emphasizing that the packaging limitation of AAV vectors continues to be a major hurdle for gene therapy of FSHD and many other diseases. To bring a CRISPRi platform for FSHD to the clinic, it was imperative to find stable repressors small enough to be included in dCas9 therapeutic cassettes, and to reduce the size of current muscle-specific regulatory cassettes.

Studies from many labs have utilized dCas9-KRAB to repress target genes; however, repression mediated by this effector requires its continuous expression. While dCas9-effectors may be continuously expressed from stable episomal AAV vectors, this is not guaranteed. From a clinical standpoint, it seems far more desirable to achieve stable repression that does not rely on continuous, lifelong expression of the transgene. Thus, a minimized cassette based on a widely used CKM-based cassette (Salva, et al. (2007) Mol Ther. 15: 320-9) was created that maintained high activity and specificity for skeletal muscles, and this FSHD-optimized cassette was used to drive expression of dSaCas9 fused to each of four small epigenetic repressors capable of mediating stable silencing. Here these examples demonstrate proof-of-principle that dSaCas9-mediated targeting of these epigenetic regulators returns the chromatin at the FSHD locus to a more normal state of repression and reduces expression of DUX4-fl and its targets in FSHD myocytes and in a DUX4-based transgenic mouse model, with minimal effects on the muscle transcriptome.

More robust repression of DUX4-fl and its targets was observed in primary FSHD myocytes than in ACTA1-MCM; FLExD bi-transgenic mice, likely due to limitations of the mouse model, which contains only a single D4Z4 repeat that may not be enough for efficient epigenetic silencing. Thus, ongoing studies will also test this CRISPRi platform in a human xenograft model containing mature FSHD myofibers (Mueller, et al. (2019) Exp. Neurol 320:113011). These mice are immunocompromised, and thus, not useful for assessing the effects of CRISPRi on DUX4-mediated immune pathologies. However, because they contain a full D4Z4 array from an FSHD patient, a xenograft model may be ideal for assessing long-term epigenetic changes at the disease locus. Determining the stability of DUX4 repression mediated by CRISPRi is a critical goal, since current AAV vectors for gene therapy can only be administered once.

A major concern of Cas9 editing is the potential for off-target cutting leading to deleterious mutations, something that was not considered to be a problem for dCas9-effectors. However, it was recently demonstrated in yeast that R-loops formed by dCas9 binding to DNA can cause mutagenesis at both on- and off-target sites (Laughery, et al. (2019) Nucleic Acids Res. 47:2389-2401), although the frequency is several orders of magnitude lower than that induced by Cas9. Consistent with this very low rate, mutations induced by dCas9 have not been detected in mammalian cells (Lei, et al. (2018) Nat. Struct. Mol. Biol. 25:45-52). Additionally, this concern is ameliorated when targeting the D4Z4 region, which is normally silent. Fortunately, for CRISPRi of FSHD, both the nature of the targeted region and the type of modulation employed tend to mitigate the general concerns related to CRISPR platforms.

As CRISPR and other gene targeting systems continue to evolve, it is important that the results of this study can be adapted to a changing platform. The identification of sgRNAs that successfully target the DUX4 locus with minimal off-target effects should prove useful with engineered Cas9 variants and dCas9 fused to other effectors. In addition, the DUX4 promoter and exon 1 have been identified as targets for epigenetic modulation, and these regions contain numerous sgRNA targets compatible with different orthologs of Cas9. Once these orthologs are better characterized, smaller and less immunogenic versions should become available, rendering fusions with larger epigenetic regulators more amenable to in vivo delivery.

These examples demonstrate the successful use of dCas9-mediated epigenetic repression in a muscle disorder, thus laying the groundwork for subsequent, ongoing studies to assess the functional efficacy and stability of this approach in vivo. Ultimately, it is important to correct the underlying pathogenic mechanism in FSHD using a therapeutically relevant platform. In addition, the successful use of dCas9-based chromatin effectors should be applicable to other diseases of aberrant gene regulation.

### Enumerated Embodiments

The following enumerated embodiments are provided, the numbering of which is not to be construed as designating levels of importance.

Embodiment 1 provides a polynucleotide encoding a CRISPR interference (CRISPRi) platform comprising a single guide RNA (sgRNA) and a fusion polypeptide, wherein the fusion polypeptide further comprises a catalytically inactive Cas9 (dCas9 or iCas9) fused to an epigenetic repressor.

Embodiment 2 provides the polynucleotide of embodiment 1, wherein the sgRNA is under control of the U6 promoter.

Embodiment 3 provides the polynucleotide of embodiment 1, wherein the sgRNA targets the DUX4 locus.

Embodiment 4 provides the polynucleotide of any one of embodiments 1-3, wherein the fusion polypeptide is under control of a skeletal muscle-specific regulatory cassette.

Embodiment 5 provides the polynucleotide of any one of embodiments 1-4, wherein the catalytically inactive Cas9 is a dSaCas9.

Embodiment 6 provides the polynucleotide of any one of embodiments 1-5, wherein the epigenetic repressor is selected from the group consisting of HP1α, HP1γ, the chromo shadow domain and C-terminal extension region of HP1α or HP1γ, MeCP2 transcription repression domain (TRD), and SUV39H1 SET domains.

Embodiment 7 provides the polynucleotide of any one of embodiment 1-6, wherein the sgRNA comprises SEQ ID NO: 38, 39, 40, 41, 42, or 43..

Embodiment 8 provides the polynucleotide of any one of embodiments 1-6, wherein the fusion polypeptide comprises any one of SEQ ID NOs: 1-4.

Embodiment 9 provides the polynucleotide of any one of embodiments 1-6, wherein the polynucleotide comprises any one of SEQ ID NOs: 48-55.

Embodiment 10 provides a vector comprising a polynucleotide encoding a CRISPRi platform comprising a sgRNA and a fusion polypeptide, wherein the fusion polypeptide further comprises a catalytically inactive Cas9 (dCas9 or iCas9) fused to an epigenetic repressor.

Embodiment 11 provides the vector of embodiment 10, wherein the sgRNA is under control of the U6 promoter.

Embodiment 12 provides the vector of embodiment 10, wherein the sgRNA targets the DUX4 locus.

Embodiment 13 provides the vector of any one of embodiments 10-12, wherein the fusion polypeptide is under control of a skeletal muscle-specific regulatory cassette.

Embodiment 14 provides the vector of any one of embodiments 10-13, wherein the catalytically inactive Cas9 is a dSaCas9.

Embodiment 15 provides the vector of any one of embodiments 10-14, wherein the epigenetic repressor is selected from the group consisting of a HP1α, HP1γ, the chromo shadow domain and C-terminal extension region of HP1α or HP1γ, MeCP2 transcription repression domain (TRD), and SUV39H1 SET domains.

Embodiment 16 provides the vector of any one of embodiments 10-15, wherein the sgRNA comprises SEQ ID NO: 38, 39, 40, 41, 42, or 43.

Embodiment 17 provides the vector of any one of embodiments 10-16, wherein the fusion polypeptide comprises any one of SEQ ID NOs: 1-4.

Embodiment 18 provides the vector of any one of embodiments 10-17, wherein the polynucleotide comprises any one of SEQ ID NOs: 48-55.

Embodiment 19 provides the vector of any one of embodiments 10-18, wherein the vector is an adeno-associated viral (AAV) vector.

Embodiment 20 provides the vector of any one of embodiments 10-19, wherein the vector comprises any one of SEQ ID NOs: 48-55.

Embodiment 21 provides a method of treating facioscapulohumeral muscular dystrophy (FSHD) in a subject in need thereof, the method comprising administering to the subject an effective amount of a repressor of DUX4 gene expression, wherein the repressor decreases DUX4 gene expression in the skeletal muscle cells of the subject, thereby treating the disorder.

Embodiment 22 provides the method of embodiment 21 wherein the DUX4 repressor is polynucleotide comprising a CRISPRi platform comprising a sgRNA and a fusion polypeptide, wherein the fusion polypeptide further comprises a dCas9 fused to an epigenetic repressor.

Embodiment 23 provides the method of any one of embodiments 21-22, wherein the sgRNA targets the DUX4 locus.

Embodiment 24 provides the method of any one of embodiments 21-23, wherein the sgRNA comprises SEQ ID NO: 38, 39, 40, 41, 42, or 43.

Embodiment 25 provides the method of any one of embodiments 21-24, wherein the dCas9 is a dSaCas9.

Embodiment 26 provides the method of any one of embodiments 21-25, wherein the epigenetic repressor is selected from the group consisting of HP1α, HP1γ, the chromo shadow domain and C-terminal extension region of HP1α or HP1γ, MeCP2 transcription repression domain (TRD), and SUV39H1 SET domains.

Embodiment 27 provides the method of any one of embodiments 21-26, wherein the fusion polypeptide is encoded by a polynucleotide comprising any one of SEQ ID NOs: 1-4.

Embodiment 28 provides the method of any one of embodiments 21-27, wherein the polynucleotide comprises any one of SEQ ID NOs: 48-55.

Embodiment 29 provides the method of any one of embodiments 21-28, wherein the subject is a mammal.

Embodiment 30 provides the method of embodiment 29, wherein the mammal is a human.

Embodiment 31 provides a method of treating FSHD in a subject in need thereof, the method comprising administering to the subject an effective amount of the vector of any one of embodiments 10-20.

Embodiment 32 provides the method of embodiment 31, wherein the subject is a mammal.

Embodiment 33 provides the method of embodiment 32, wherein the mammal is a human.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiment or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A polynucleotide encoding a CRISPR interference (CRISPRi) platform comprising a single guide RNA (sgRNA) and a fusion polypeptide, wherein the fusion polypeptide further comprises a catalytically inactive Cas9 (dCas9 or iCas9) fused to an epigenetic repressor.

2. The polynucleotide of claim 1, wherein the sgRNA is under control of the U6 promoter.

3. The polynucleotide of claim 1, wherein the sgRNA targets the DUX4 locus.

4. The polynucleotide of claim 1, wherein the fusion polypeptide is under control of a skeletal muscle-specific regulatory cassette.

5. The polynucleotide of claim 1, wherein the catalytically inactive Cas9 is a dSaCas9.

6. The polynucleotide of claim 1, wherein the epigenetic repressor is selected from the group consisting of HP1α, HP1γ, the chromo shadow domain and C-terminal extension region of HP1α or HP1γ, MeCP2 transcription repression domain (TRD), and SUV39H1 SET domain.

7. The polynucleotide of claim 1, wherein the sgRNA comprises a nucleotide sequence selected from the group consisting of SEQ ID NOs: 38, 39, 40, 41, 42, and 43.

8. The polynucleotide of claim 1, wherein the fusion polypeptide comprises an amino acid sequence selected from the group consisting of SEQ ID NOs: 1-4.

9. The polynucleotide of claim 1, wherein the polynucleotide comprises an amino acid sequence selected from the group consisting of SEQ ID NOs: 48-55.

10. A vector comprising a polynucleotide encoding a CRISPRi platform comprising a sgRNA and a fusion polypeptide, wherein the fusion polypeptide further comprises a catalytically inactive Cas9 (dCas9 or iCas9) fused to an epigenetic repressor.

11. The vector of claim 10, wherein the sgRNA is under control of the U6 promoter.

12. The vector of claim 10, wherein the sgRNA targets the DUX4 locus.

13. The vector of claim 10, wherein the fusion polypeptide is under control of a skeletal muscle-specific regulatory cassette.

14. The vector of claim 10, wherein the catalytically inactive Cas9 is a dSaCas9.

15. The vector of claim 10, wherein the epigenetic repressor is selected from the group consisting of HP1α, HP1γ, the chromo shadow domain and C-terminal extension region of HP1α or HP1γ, MeCP2 transcription repression domain (TRD), and SUV39H1 SET domain.

16. The vector of claim 10, wherein the sgRNA comprises a nucleotide sequence selected from the group consisting of SEQ ID NOs: 38, 39, 40, 41, 42, and 43.

17. The vector of claim 10, wherein the fusion polypeptide comprises an amino acid sequence selected from the group consisting of SEQ ID NOs: 1-4.

18. The vector of claim 10, wherein the polynucleotide comprises an amino acid sequence selected from the group consisting of SEQ ID NOs: 48-55.

19. The vector of claim 10, wherein the vector is an adeno-associated viral (AAV) vector.

20. The vector of claim 10, wherein the vector comprises a nucleotide sequence selected from the group consisting of SEQ ID NOs: 48-55.

21. A method of treating facioscapulohumeral muscular dystrophy (FSHD) in a subject in need thereof, the method comprising administering to the subject an effective amount of a repressor of DUX4 gene expression, wherein the repressor decreases DUX4 gene expression in the skeletal muscle cells of the subject, thereby treating the disorder.

22. The method of claim 21 wherein the DUX4 repressor is polynucleotide comprising a CRISPRi platform comprising a sgRNA and a fusion polypeptide, wherein the fusion polypeptide further comprises a dCas9 fused to an epigenetic repressor.

23. The method of claim 21, wherein the sgRNA targets the DUX4 locus.

24. The method of claim 21, wherein the sgRNA comprises a nucleotide sequence selected from the group consisting of SEQ ID NO: 38, 39, 40, 41, 42, and 43.

25. The method of claim 21, wherein the dCas9 is a dSaCas9.

26. The method of claim 21, wherein the epigenetic repressor is selected from the group consisting of HP1α, HP1γ, the chromo shadow domain and C-terminal extension region of HP1α or HP1γ, MeCP2 transcription repression domain (TRD), and SUV39H1 SET domains.

27. The method of claim 21, wherein the fusion polypeptide is encoded by a polynucleotide comprising a nucleotide sequence selected from the group consisting of SEQ ID NOs: 1-4.

28. The method of claim 21, wherein the polynucleotide comprises an amino acid sequence selected from the group consisting of SEQ ID NOs: 48-55.

29. The method of claim 21, wherein the subject is a mammal.

30. The method of claim 29, wherein the mammal is a human.

31. A method of treating FSHD in a subject in need thereof, the method comprising administering to the subject an effective amount of the vector of claim 10.

32. The method of claim 31, wherein the subject is a mammal.

33. The method of claim 32, wherein the mammal is a human.


SEQUENCE LISTING (58 sequences)

SEQ ID NO: 1 - protein, 1191 aa, Artificial Sequence
  SUV39H1 SET dSaCas9 Fusion Protein
MAPKKKRKVEASKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGGTGGPKKKRKVGRAYDLCIFRTDDGRGWGVRTLEKIRKNSFVMEYVGEIITSEEAERRGQIYDRQGATYLFDLDYVEDVYTVDAAYYGNISHFVNHSCDPNLQVYNVFIDNLDERLPRYPYDVPDYA

SEQ ID NO: 2 - protein, 1128 aa, Artificial Sequence
  MeCP2 TRD dSaCas9 Fusion Protein
MAPKKKRKVEASKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGGTGGPKKKRKVGRAGRKPGSVVAAAAAEAKKKAVKESSIRSVQETVLPIKKRKTRYPYDVPDYA

SEQ ID NO: 3 - protein, 1158 aa, Artificial Sequence
  HP1alpha chromo shadow and CTE dSaCas9 Fusion Protein
MAPKKKRKVEASKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGGTGGPKKKRKVGRALEPEKIIGATDSCGDLMFLMKWKDTDEADLVLAKEANVKCPQIVIAFYEERLTWHAYPEDAENKEKETAKSYPYDVPDYA

SEQ ID NO: 4 - protein, 1150 aa, Artificial Sequence
  HP1gamma chromo shadow and CTE dSaCas9 Fusion Protein
MAPKKKRKVEASKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGGTGGPKKKRKVGRALDPERIIGATDSSGELMFLMKWKDSDEADLVLAKEANMKCPQIVIAFYEERLTWHSCPEDEAQYPYDVPDYA

SEQ ID NO: 5 - DNA, 27 bp, Artificial Sequence
  DUX4 prom
cggccccaggcctcgacgccctggggt

SEQ ID NO: 6 - DNA, 26 bp, Artificial Sequence
  LAAT1
aggccccaggctcgccgccccaggat

SEQ ID NO: 7 - DNA, 27 bp, Artificial Sequence
  DUX4 exon 1
ctgtgcagcgcggcccccggcgggggt

SEQ ID NO: 8 - DNA, 25 bp, Artificial Sequence
  RRS1
ctgtagctcggcctccggcgtgggt

SEQ ID NO: 9 - DNA, 25 bp, Artificial Sequence
  GNAI1
ctgcggcgcggccaccggcgggagt

SEQ ID NO: 10 - DNA, 23 bp, Artificial Sequence
  DUX4-fl-F
gctctgctggaggagctttagga

SEQ ID NO: 11 - DNA, 24 bp, Artificial Sequence
  DUX4-fl-R
cgcactgctcgcaggtctgcwggt

SEQ ID NO: 12 - DNA, 24 bp, Artificial Sequence
  DUX4-fl-nested-F
agctttaggacgcggggttgggac

SEQ ID NO: 13 - DNA, 20 bp, Artificial Sequence
  DUX4-fl-nested-R
gcaggtctgcwggtacctgg

SEQ ID NO: 14 - DNA, 20 bp, Artificial Sequence
  TRIM43-F
acccatcactggactggtgt

SEQ ID NO: 15 - DNA, 20 bp, Artificial Sequence
  TRIM43-R:
cacatcctcaaagagcctga

SEQ ID NO: 16 - DNA, 20 bp, Artificial Sequence
  MBD3L2-F:
gcgttcacctcttttccaag

SEQ ID NO: 17 - DNA, 20 bp, Artificial Sequence
  MBD3L2-R:
gccatgtggatttctcgttt

SEQ ID NO: 18 - DNA, 20 bp, Artificial Sequence
  MYH1-F:
acagaagcgcaatgttgaag

SEQ ID NO: 19 - DNA, 20 bp, Artificial Sequence
  MYH1-R
cacctttgcttgcagtttgt

SEQ ID NO: 20 - DNA, 22 bp, Artificial Sequence
  FRG1-F
tctacagagacgtaggctgtca

SEQ ID NO: 21 - DNA, 20 bp, Artificial Sequence
  FRG1-R
cttgagcacgagcttggtag

SEQ ID NO: 22 - DNA, 18 bp, Artificial Sequence
  FRG2-F
gggaaaactgcaggaaaa

SEQ ID NO: 23 - DNA, 21 bp, Artificial Sequence
  FRG2-R
ctggacagttccctgctgtgt

SEQ ID NO: 24 - DNA, 20 bp, Artificial Sequence
  LAAT1-F
tctgctttgctgcatctacc

SEQ ID NO: 25 - DNA, 20 bp, Artificial Sequence
  LAAT1-R
agtacagcgtcagcatcacc

SEQ ID NO: 26 - DNA, 20 bp, Artificial Sequence
  RRS1-F
cacaaccgagactttggaga

SEQ ID NO: 27 - DNA, 20 bp, Artificial Sequence
  RRS1-R
tcccgctctgatacacaaac

SEQ ID NO: 28 - DNA, 20 bp, Artificial Sequence
  GNAI1-F
catcccgactcaacaagatg

SEQ ID NO: 29 - DNA, 20 bp, Artificial Sequence
  GNAI1-R
tgcattcggttcatttcttc

SEQ ID NO: 30 - DNA, 20 bp, Artificial Sequence
  DUX4 prom-F
cctgttgctcacgtctctcc

SEQ ID NO: 31 - DNA, 19 bp, Artificial Sequence
  DUX4 prom-R
gtggggagtctgcagtgtg

SEQ ID NO: 32 - DNA, 18 bp, Artificial Sequence
  DUX4 TSS-F
gacaccctcggacagcac

SEQ ID NO: 33 - DNA, 19 bp, Artificial Sequence
  DUX4 TSS-R
gtacgggttccgctcaaag

SEQ ID NO: 34 - DNA, 18 bp, Artificial Sequence
  DUX4 exon3-F
ctgacgtgcaagggagct

SEQ ID NO: 35 - DNA, 19 bp, Artificial Sequence
  DUX4 exon3-R
caggtttgcctagacagcg

SEQ ID NO: 36 - DNA, 19 bp, Artificial Sequence
  4-spec D4Z4-F
tctgctggaggagctttag

SEQ ID NO: 37 - DNA, 18 bp, Artificial Sequence
  4-spec D4Z4-R
gaatggcagttctccgcg

SEQ ID NO: 38 - DNA, 21 bp, Artificial Sequence
  sgRNA-1
cggccccaggcctcgacgccc

SEQ ID NO: 39 - DNA, 21 bp, Artificial Sequence
  sgRNA-2
tcgacgccctggggtcccttc

SEQ ID NO: 40 - DNA, 21 bp, Artificial Sequence
  sgRNA-3
tccgcggggagggtgctgtcc

SEQ ID NO: 41 - DNA, 21 bp, Artificial Sequence
  sgRNA-4
gccagctgaggcagcaccggc

SEQ ID NO: 42 - DNA, 21 bp, Artificial Sequence
  sgRNA-5
ctgtgcagcgcggcccccggc

SEQ ID NO: 43 - DNA, 21 bp, Artificial Sequence
  sgRNA-6
tcatccagcagcaggccgcag

SEQ ID NO: 44 - DNA, 23 bp, Artificial Sequence
  bGH-F
tctagttgccagccatctgttgt

SEQ ID NO: 45 - DNA, 18 bp, Artificial Sequence
  bGH-R
tgggagtggcaccttcca

SEQ ID NO: 46 - DNA, 28 bp, Artificial Sequence
  Rosa26-F
caatacctttctgggagttctctgctgc

SEQ ID NO: 47 - DNA, 22 bp, Artificial Sequence
  Rosa26-R
tgcaggacaacgcccacacacc

SEQ ID NO: 48 - DNA, 4388 bp, Artificial Sequence
  AIO CLH-17v2 (mouse CKM-TRD)
  misc_feature (4263)..(4283): n is a, c, g, or t
acgcgtgatatcggacacccgagatgcctggttataattaacccagacatgtggctgccccccccaacacctgctgcgagctctaaaaataaccctgggacacccgagatgcctggttataattaacccagacatgtggctgccccccccaacacctgctgcgagctctaaaaataaccctgggacacccgagatgcctggttataattaacccagacatgtggctgccccccccaacacctgctgcgagctctaaaaataacccctccctggggacagcccctcctggctagtcacaccctgtaggctcctctatataacccaggggcacaggggctgccctcattctaccaccacctccacagcacagacagacactcaggagccagccagcgccaccatggcccccaagaagaagaggaaggtggaggccagcaagcggaactacatcctgggcctggccatcggcatcaccagcgtgggctacggcatcatcgactacgagacccgggacgtgatcgacgccggcgtgcggctgttcaaggaggccaacgtggagaacaacgagggcaggcggagcaagagaggcgccagaaggctgaagcggcggaggcggcacagaatccagagagtgaagaagctgctgttcgactacaacctgctgaccgaccacagcgagctgagcggcatcaacccctacgaggccagagtgaagggcctgagccagaagctgagcgaggaggagttcagcgccgccctgctgcacctggccaagagaagaggcgtgcacaacgtgaacgaggtggaggaggacaccggcaacgagctgtccaccaaggagcagatcagccggaacagcaaggccctggaggagaagtacgtggccgagctgcagctggagcggctgaagaaggacggcgaggtgcggggcagcatcaacagattcaagaccagcgactacgtgaaggaggccaagcagctgctgaaggtgcagaaggcctaccaccagctggaccagagcttcatcgacacctacatcgacctgctggagacccggcggacctactacgagggccccggcgagggcagccccttcggctggaaggacatcaaggagtggtacgagatgctgatgggccactgcacctacttccccgaggagctgcggagcgtgaagtacgcctacaacgccgacctgtacaacgccctgaacgacctgaacaacctggtgatcaccagggacgagaacgagaagctggagtactacgagaagttccagatcatcgagaacgtgttcaagcagaagaagaagcccaccctgaagcagatcgccaaggagatcctggtgaacgaggaggacatcaagggctacagagtgaccagcaccggcaagcccgagttcaccaacctgaaggtgtaccacgacatcaaggacatcaccgcccggaaggagatcatcgagaacgccgagctgctggaccagatcgccaagatcctgaccatctaccagagcagcgaggacatccaggaggagctgaccaacctgaactccgagctgacccaggaggagatcgagcagatcagcaacctgaagggctacaccggcacccacaacctgagcctgaaggccatcaacctgatcctggacgagctgtggcacaccaacgacaaccagatcgccatcttcaaccggctgaagctggtgcccaagaaggtggacctgtcccagcagaaggagatccccaccaccctggtggacgacttcatcctgagccccgtggtgaagagaagcttcatccagagcatcaaggtgatcaacgccatcatcaagaagtacggcctgcccaacgacatcatcatcgagctggcccgggagaagaactccaaggacgcccagaagatgatcaacgagatgcagaagcggaaccggcagaccaacgagcggatcgaggagatcatccggaccaccggcaaggagaacgccaagtacctgatcgagaagatcaagctgcacgacatgcaggagggcaagtgcctgtacagcctggaggccatccccctggaggacctgctgaacaaccccttcaactacgaggtggaccacatcatccccagaagcgtgtccttcgacaacagcttcaacaacaaggtgctggtgaagcaggaggaggccagcaagaagggcaaccggacccccttccagtacctgagcagcagcgacagcaagatcagctacgagaccttcaagaagcacatcctgaacctggccaagggcaagggcagaatcagcaagaccaagaaggagtacctgctggaggagcgggacatcaacaggttctccgtgcagaaggacttcatcaaccggaacctggtggacaccagatacgccaccagaggcctgatgaacctgctgcggagctacttcagagtgaacaacctggacgtgaaggtgaagtccatcaacggcggcttcaccagcttcctgcggcggaagtggaagttcaagaaggagcggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccaccgggtggacaagaagcccaacagagagctgatcaacgacaccctgtactccacccggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtactccaagaaggacaacggccccgtgatcaagaagatcaagtactacggcaacaagctgaacgcccacctggacatcaccgacgactaccccaacagcagaaacaaggtggtgaagctgtccctgaagccctacagattcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgcctccttctacaacaacgacctgatcaagatcaacggcgagctgtacagagtgatcggcgtgaacaacgacctgctgaaccggatcgaggtgaacatgatcgacatcacctaccgcgagtacctggagaacatgaacgacaagaggccccccaggatcatcaagaccatcgcctccaagacccagagcatcaagaagtacagcaccgacatcctgggcaacctgtacgaggtgaagtccaagaagcacccccagatcatcaagaagggcggcaccggcggccccaagaagaagaggaaggtgggccgggccggccggaagcccggcagcgtggtggccgccgccgccgccgaggccaagaagaaggccgtgaaggagagcagcatccggagcgtgcaggagaccgtgctgcccatcaagaagcggaagaccagatacccctacgacgtgcccgactacgcctgatatttgtgaaatttgtgatgctattgctttatttgtaaccatctagctttatttgtgaaatttgtgatgctattgctttatttgtaaccattttatttgtgaaatttgtgatgctattgctttatttgtaaccattataagctgcaataaacaagttaacaacaacaattgcattcattttatgtttcaggttcagggggagatgtgggaggttttttaaagcgggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnnngtttaagtactctgtgctggaaacagcacagaatctacttaaacaaggcaaaatgccgtgtttatctcgtcaacttgttggcgagatttttttggtaccggaccg

SEQ ID NO: 49 - DNA, 4385 bp, Artificial Sequence
  AIO CLH-17v2 (human CKM-TRD)
  misc_feature (4260)..(4280): n is a, c, g, or t
acgcgtgatatcggacacccgagacgcccggttataattaaccaggacacgtggcgaccccccccaacacctgcccgacctctaaaaataactcctggacacccgagacgcccggttataattaaccaggacacgtggcgaccccccccaacacctgcccgacctctaaaaataactcctggacacccgagacgcccggttataattaaccaggacacgtggcgaccccccccaacacctgcccgacctctaaaaataacccctccctggggacaacccctcccagccaatagcacagcctaggtccccctatataaggccacggctgctggcccttcctcattctcagtgtcacctccaggatacagacagccccccttcagcccagcccgccaccatggcccccaagaagaagaggaaggtggaggccagcaagcggaactacatcctgggcctggccatcggcatcaccagcgtgggctacggcatcatcgactacgagacccgggacgtgatcgacgccggcgtgcggctgttcaaggaggccaacgtggagaacaacgagggcaggcggagcaagagaggcgccagaaggctgaagcggcggaggcggcacagaatccagagagtgaagaagctgctgttcgactacaacctgctgaccgaccacagcgagctgagcggcatcaacccctacgaggccagagtgaagggcctgagccagaagctgagcgaggaggagttcagcgccgccctgctgcacctggccaagagaagaggcgtgcacaacgtgaacgaggtggaggaggacaccggcaacgagctgtccaccaaggagcagatcagccggaacagcaaggccctggaggagaagtacgtggccgagctgcagctggagcggctgaagaaggacggcgaggtgcggggcagcatcaacagattcaagaccagcgactacgtgaaggaggccaagcagctgctgaaggtgcagaaggcctaccaccagctggaccagagcttcatcgacacctacatcgacctgctggagacccggcggacctactacgagggccccggcgagggcagccccttcggctggaaggacatcaaggagtggtacgagatgctgatgggccactgcacctacttccccgaggagctgcggagcgtgaagtacgcctacaacgccgacctgtacaacgccctgaacgacctgaacaacctggtgatcaccagggacgagaacgagaagctggagtactacgagaagttccagatcatcgagaacgtgttcaagcagaagaagaagcccaccctgaagcagatcgccaaggagatcctggtgaacgaggaggacatcaagggctacagagtgaccagcaccggcaagcccgagttcaccaacctgaaggtgtaccacgacatcaaggacatcaccgcccggaaggagatcatcgagaacgccgagctgctggaccagatcgccaagatcctgaccatctaccagagcagcgaggacatccaggaggagctgaccaacctgaactccgagctgacccaggaggagatcgagcagatcagcaacctgaagggctacaccggcacccacaacctgagcctgaaggccatcaacctgatcctggacgagctgtggcacaccaacgacaaccagatcgccatcttcaaccggctgaagctggtgcccaagaaggtggacctgtcccagcagaaggagatccccaccaccctggtggacgacttcatcctgagccccgtggtgaagagaagcttcatccagagcatcaaggtgatcaacgccatcatcaagaagtacggcctgcccaacgacatcatcatcgagctggcccgggagaagaactccaaggacgcccagaagatgatcaacgagatgcagaagcggaaccggcagaccaacgagcggatcgaggagatcatccggaccaccggcaaggagaacgccaagtacctgatcgagaagatcaagctgcacgacatgcaggagggcaagtgcctgtacagcctggaggccatccccctggaggacctgctgaacaaccccttcaactacgaggtggaccacatcatccccagaagcgtgtccttcgacaacagcttcaacaacaaggtgctggtgaagcaggaggaggccagcaagaagggcaaccggacccccttccagtacctgagcagcagcgacagcaagatcagctacgagaccttcaagaagcacatcctgaacctggccaagggcaagggcagaatcagcaagaccaagaaggagtacctgctggaggagcgggacatcaacaggttctccgtgcagaaggacttcatcaaccggaacctggtggacaccagatacgccaccagaggcctgatgaacctgctgcggagctacttcagagtgaacaacctggacgtgaaggtgaagtccatcaacggcggcttcaccagcttcctgcggcggaagtggaagttcaagaaggagcggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccaccgggtggacaagaagcccaacagagagctgatcaacgacaccctgtactccacccggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtactccaagaaggacaacggccccgtgatcaagaagatcaagtactacggcaacaagctgaacgcccacctggacatcaccgacgactaccccaacagcagaaacaaggtggtgaagctgtccctgaagccctacagattcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgcctccttctacaacaacgacctgatcaagatcaacggcgagctgtacagagtgatcggcgtgaacaacgacctgctgaaccggatcgaggtgaacatgatcgacatcacctaccgcgagtacctggagaacatgaacgacaagaggccccccaggatcatcaagaccatcgcctccaagacccagagcatcaagaagtacagcaccgacatcctgggcaacctgtacgaggtgaagtccaagaagcacccccagatcatcaagaagggcggcaccggcggccccaagaagaagaggaaggtgggccgggccggccggaagcccggcagcgtggtggccgccgccgccgccgaggccaagaagaaggccgtgaaggagagcagcatccggagcgtgcaggagaccgtgctgcccatcaagaagcggaagaccagatacccctacgacgtgcccgactacgcctgatatttgtgaaatttgtgatgctattgctttatttgtaaccatctagctttatttgtgaaatttgtgatgctattgctttatttgtaaccattttatttgtgaaatttgtgatgctattgctttatttgtaaccattataagctgcaataaacaagttaacaacaacaattgcattcattttatgtttcaggttcagggggagatgtgggaggttttttaaagcgggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnnngtttaagtactctgtgctggaaacagcacagaatctacttaaacaaggcaaaatgccgtgtttatctcgtcaacttgttggcgagatttttttggtaccggaccg

SEQ ID NO: 50 - DNA, 4478 bp, Artificial Sequence
  AIO CLH-22 (mouse CKM-HP1alpha)
  misc_feature (4353)..(4373): n is a, c, g, or t
acgcgtgatatcggacacccgagatgcctggttataattaacccagacatgtggctgccccccccaacacctgctgcgagctctaaaaataaccctgggacacccgagatgcctggttataattaacccagacatgtggctgccccccccaacacctgctgcgagctctaaaaataaccctgggacacccgagatgcctggttataattaacccagacatgtggctgccccccccaacacctgctgcgagctctaaaaataacccctccctggggacagcccctcctggctagtcacaccctgtaggctcctctatataacccaggggcacaggggctgccctcattctaccaccacctccacagcacagacagacactcaggagccagccagcgccaccatggcccccaagaagaagaggaaggtggaggccagcaagcggaactacatcctgggcctggccatcggcatcaccagcgtgggctacggcatcatcgactacgagacccgggacgtgatcgacgccggcgtgcggctgttcaaggaggccaacgtggagaacaacgagggcaggcggagcaagagaggcgccagaaggctgaagcggcggaggcggcacagaatccagagagtgaagaagctgctgttcgactacaacctgctgaccgaccacagcgagctgagcggcatcaacccctacgaggccagagtgaagggcctgagccagaagctgagcgaggaggagttcagcgccgccctgctgcacctggccaagagaagaggcgtgcacaacgtgaacgaggtggaggaggacaccggcaacgagctgtccaccaaggagcagatcagccggaacagcaaggccctggaggagaagtacgtggccgagctgcagctggagcggctgaagaaggacggcgaggtgcggggcagcatcaacagattcaagaccagcgactacgtgaaggaggccaagcagctgctgaaggtgcagaaggcctaccaccagctggaccagagcttcatcgacacctacatcgacctgctggagacccggcggacctactacgagggccccggcgagggcagccccttcggctggaaggacatcaaggagtggtacgagatgctgatgggccactgcacctacttccccgaggagctgcggagcgtgaagtacgcctacaacgccgacctgtacaacgccctgaacgacctgaacaacctggtgatcaccagggacgagaacgagaagctggagtactacgagaagttccagatcatcgagaacgtgttcaagcagaagaagaagcccaccctgaagcagatcgccaaggagatcctggtgaacgaggaggacatcaagggctacagagtgaccagcaccggcaagcccgagttcaccaacctgaaggtgtaccacgacatcaaggacatcaccgcccggaaggagatcatcgagaacgccgagctgctggaccagatcgccaagatcctgaccatctaccagagcagcgaggacatccaggaggagctgaccaacctgaactccgagctgacccaggaggagatcgagcagatcagcaacctgaagggctacaccggcacccacaacctgagcctgaaggccatcaacctgatcctggacgagctgtggcacaccaacgacaaccagatcgccatcttcaaccggctgaagctggtgcccaagaaggtggacctgtcccagcagaaggagatccccaccaccctggtggacgacttcatcctgagccccgtggtgaagagaagcttcatccagagcatcaaggtgatcaacgccatcatcaagaagtacggcctgcccaacgacatcatcatcgagctggcccgggagaagaactccaaggacgcccagaagatgatcaacgagatgcagaagcggaaccggcagaccaacgagcggatcgaggagatcatccggaccaccggcaaggagaacgccaagtacctgatcgagaagatcaagctgcacgacatgcaggagggcaagtgcctgtacagcctggaggccatccccctggaggacctgctgaacaaccccttcaactacgaggtggaccacatcatccccagaagcgtgtccttcgacaacagcttcaacaacaaggtgctggtgaagcaggaggaggccagcaagaagggcaaccggacccccttccagtacctgagcagcagcgacagcaagatcagctacgagaccttcaagaagcacatcctgaacctggccaagggcaagggcagaatcagcaagaccaagaaggagtacctgctggaggagcgggacatcaacaggttctccgtgcagaaggacttcatcaaccggaacctggtggacaccagatacgccaccagaggcctgatgaacctgctgcggagctacttcagagtgaacaacctggacgtgaaggtgaagtccatcaacggcggcttcaccagcttcctgcggcggaagtggaagttcaagaaggagcggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccaccgggtggacaagaagcccaacagagagctgatcaacgacaccctgtactccacccggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtactccaagaaggacaacggccccgtgatcaagaagatcaagtactacggcaacaagctgaacgcccacctggacatcaccgacgactaccccaacagcagaaacaaggtggtgaagctgtccctgaagccctacagattcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgcctccttctacaacaacgacctgatcaagatcaacggcgagctgtacagagtgatcggcgtgaacaacgacctgctgaaccggatcgaggtgaacatgatcgacatcacctaccgcgagtacctggagaacatgaacgacaagaggccccccaggatcatcaagaccatcgcctccaagacccagagcatcaagaagtacagcaccgacatcctgggcaacctgtacgaggtgaagtccaagaagcacccccagatcatcaagaagggcggcaccggcggccccaagaagaagaggaaggtgggccgggccctggagcccgagaagatcatcggcgccaccgactcctgcggcgacctgatgttcctgatgaagtggaaggacaccgacgaggccgacctggtgctggccaaggaggccaacgtgaagtgcccccagatcgtgatcgccttctacgaggagcggctgacctggcacgcctaccccgaggacgccgagaacaaggagaaggagaccgccaagagctacccctacgacgtgcccgactacgcctgatatttgtgaaatttgtgatgctattgctttatttgtaaccatctagctttatttgtgaaatttgtgatgctattgctttatttgtaaccattttatttgtgaaatttgtgatgctattgctttatttgtaaccattataagctgcaataaacaagttaacaacaacaattgcattcattttatgtttcaggttcagggggagatgtgggaggttttttaaagcgggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnnngtttaagtactctgtgctggaaacagcacagaatctacttaaacaaggcaaaatgccgtgtttatctcgtcaacttgttggcgagatttttttggtaccggaccg

SEQ ID NO: 51 - DNA, 4475 bp, Artificial Sequence
  AIO CLH-22 (human CKM-HP1alpha)
  misc_feature (4350)..(4370): n is a, c, g, or t
acgcgtgatatcggacacccgagacgcccggttataattaaccaggacacgtggcgaccccccccaacacctgcccgacctctaaaaataactcctggacacccgagacgcccggttataattaaccaggacacgtggcgaccccccccaacacctgcccgacctctaaaaataactcctggacacccgagacgcccggttataattaaccaggacacgtggcgaccccccccaacacctgcccgacctctaaaaataacccctccctggggacaacccctcccagccaatagcacagcctaggtccccctatataaggccacggctgctggcccttcctcattctcagtgtcacctccaggatacagacagccccccttcagcccagcccgccaccatggcccccaagaagaagaggaaggtggaggccagcaagcggaactacatcctgggcctggccatcggcatcaccagcgtgggctacggcatcatcgactacgagacccgggacgtgatcgacgccggcgtgcggctgttcaaggaggccaacgtggagaacaacgagggcaggcggagcaagagaggcgccagaaggctgaagcggcggaggcggcacagaatccagagagtgaagaagctgctgttcgactacaacctgctgaccgaccacagcgagctgagcggcatcaacccctacgaggccagagtgaagggcctgagccagaagctgagcgaggaggagttcagcgccgccctgctgcacctggccaagagaagaggcgtgcacaacgtgaacgaggtggaggaggacaccggcaacgagctgtccaccaaggagcagatcagccggaacagcaaggccctggaggagaagtacgtggccgagctgcagctggagcggctgaagaaggacggcgaggtgcggggcagcatcaacagattcaagaccagcgactacgtgaaggaggccaagcagctgctgaaggtgcagaaggcctaccaccagctggaccagagcttcatcgacacctacatcgacctgctggagacccggcggacctactacgagggccccggcgagggcagccccttcggctggaaggacatcaaggagtggtacgagatgctgatgggccactgcacctacttccccgaggagctgcggagcgtgaagtacgcctacaacgccgacctgtacaacgccctgaacgacctgaacaacctggtgatcaccagggacgagaacgagaagctggagtactacgagaagttccagatcatcgagaacgtgttcaagcagaagaagaagcccaccctgaagcagatcgccaaggagatcctggtgaacgaggaggacatcaagggctacagagtgaccagcaccggcaagcccgagttcaccaacctgaaggtgtaccacgacatcaaggacatcaccgcccggaaggagatcatcgagaacgccgagctgctggaccagatcgccaagatcctgaccatctaccagagcagcgaggacatccaggaggagctgaccaacctgaactccgagctgacccaggaggagatcgagcagatcagcaacctgaagggctacaccggcacccacaacctgagcctgaaggccatcaacctgatcctggacgagctgtggcacaccaacgacaaccagatcgccatcttcaaccggctgaagctggtgcccaagaaggtggacctgtcccagcagaaggagatccccaccaccctggtggacgacttcatcctgagccccgtggtgaagagaagcttcatccagagcatcaaggtgatcaacgccatcatcaagaagtacggcctgcccaacgacatcatcatcgagctggcccgggagaagaactccaaggacgcccagaagatgatcaacgagatgcagaagcggaaccggcagaccaacgagcggatcgaggagatcatccggaccaccggcaaggagaacgccaagtacctgatcgagaagatcaagctgcacgacatgcaggagggcaagtgcctgtacagcctggaggccatccccctggaggacctgctgaacaaccccttcaactacgaggtggaccacatcatccccagaagcgtgtccttcgacaacagcttcaacaacaaggtgctggtgaagcaggaggaggccagcaagaagggcaaccggacccccttccagtacctgagcagcagcgacagcaagatcagctacgagaccttcaagaagcacatcctgaacctggccaagggcaagggcagaatcagcaagaccaagaaggagtacctgctggaggagcgggacatcaacaggttctccgtgcagaaggacttcatcaaccggaacctggtggacaccagatacgccaccagaggcctgatgaacctgctgcggagctacttcagagtgaacaacctggacgtgaaggtgaagtccatcaacggcggcttcaccagcttcctgcggcggaagtggaagttcaagaaggagcggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccaccgggtggacaagaagcccaacagagagctgatcaacgacaccctgtactccacccggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtactccaagaaggacaacggccccgtgatcaagaagatcaagtactacggcaacaagctgaacgcccacctggacatcaccgacgactaccccaacagcagaaacaaggtggtgaagctgtccctgaagccctacagattcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgcctccttctacaacaacgacctgatcaagatcaacggcgagctgtacagagtgatcggcgtgaacaacgacctgctgaaccggatcgaggtgaacatgatcgacatcacctaccgcgagtacctggagaacatgaacgacaagaggccccccaggatcatcaagaccatcgcctccaagacccagagcatcaagaagtacagcaccgacatcctgggcaacctgtacgaggtgaagtccaagaagcacccccagatcatcaagaagggcggcaccggcggccccaagaagaagaggaaggtgggccgggccctggagcccgagaagatcatcggcgccaccgactcctgcggcgacctgatgttcctgatgaagtggaaggacaccgacgaggccgacctggtgctggccaaggaggccaacgtgaagtgcccccagatcgtgatcgccttctacgaggagcggctgacctggcacgcctaccccgaggacgccgagaacaaggagaaggagaccgccaagagctacccctacgacgtgcccgactacgcctgatatttgtgaaatttgtgatgctattgctttatttgtaaccatctagctttatttgtgaaatttgtgatgctattgctttatttgtaaccattttatttgtgaaatttgtgatgctattgctttatttgtaaccattataagctgcaataaacaagttaacaacaacaattgcattcattttatgtttcaggttcagggggagatgtgggaggttttttaaagcgggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnnngtttaagtactctgtgctggaaacagcacagaatctacttaaacaaggcaaaatgccgtgtttatctcgtcaacttgttggcgagatttttttggtaccggaccg

SEQ ID NO: 52 - DNA, 4454 bp, Artificial Sequence
  AIO CLH-23 (mouse CKM-HP1gamma)
  misc_feature (4329)..(4349): n is a, c, g, or t
acgcgtgatatcggacacccgagatgcctggttataattaacccagacatgtggctgccccccccaacacctgctgcgagctctaaaaataaccctgggacacccgagatgcctggttataattaacccagacatgtggctgccccccccaacacctgctgcgagctctaaaaataaccctgggacacccgagatgcctggttataattaacccagacatgtggctgccccccccaacacctgctgcgagctctaaaaataacccctccctggggacagcccctcctggctagtcacaccctgtaggctcctctatataacccaggggcacaggggctgccctcattctaccaccacctccacagcacagacagacactcaggagccagccagcgccaccatggcccccaagaagaagaggaaggtggaggccagcaagcggaactacatcctgggcctggccatcggcatcaccagcgtgggctacggcatcatcgactacgagacccgggacgtgatcgacgccggcgtgcggctgttcaaggaggccaacgtggagaacaacgagggcaggcggagcaagagaggcgccagaaggctgaagcggcggaggcggcacagaatccagagagtgaagaagctgctgttcgactacaacctgctgaccgaccacagcgagctgagcggcatcaacccctacgaggccagagtgaagggcctgagccagaagctgagcgaggaggagttcagcgccgccctgctgcacctggccaagagaagaggcgtgcacaacgtgaacgaggtggaggaggacaccggcaacgagctgtccaccaaggagcagatcagccggaacagcaaggccctggaggagaagtacgtggccgagctgcagctggagcggctgaagaaggacggcgaggtgcggggcagcatcaacagattcaagaccagcgactacgtgaaggaggccaagcagctgctgaaggtgcagaaggcctaccaccagctggaccagagcttcatcgacacctacatcgacctgctggagacccggcggacctactacgagggccccggcgagggcagccccttcggctggaaggacatcaaggagtggtacgagatgctgatgggccactgcacctacttccccgaggagctgcggagcgtgaagtacgcctacaacgccgacctgtacaacgccctgaacgacctgaacaacctggtgatcaccagggacgagaacgagaagctggagtactacgagaagttccagatcatcgagaacgtgttcaagcagaagaagaagcccaccctgaagcagatcgccaaggagatcctggtgaacgaggaggacatcaagggctacagagtgaccagcaccggcaagcccgagttcaccaacctgaaggtgtaccacgacatcaaggacatcaccgcccggaaggagatcatcgagaacgccgagctgctggaccagatcgccaagatcctgaccatctaccagagcagcgaggacatccaggaggagctgaccaacctgaactccgagctgacccaggaggagatcgagcagatcagcaacctgaagggctacaccggcacccacaacctgagcctgaaggccatcaacctgatcctggacgagctgtggcacaccaacgacaaccagatcgccatcttcaaccggctgaagctggtgcccaagaaggtggacctgtcccagcagaaggagatccccaccaccctggtggacgacttcatcctgagccccgtggtgaagagaagcttcatccagagcatcaaggtgatcaacgccatcatcaagaagtacggcctgcccaacgacatcatcatcgagctggcccgggagaagaactccaaggacgcccagaagatgatcaacgagatgcagaagcggaaccggcagaccaacgagcggatcgaggagatcatccggaccaccggcaaggagaacgccaagtacctgatcgagaagatcaagctgcacgacatgcaggagggcaagtgcctgtacagcctggaggccatccccctggaggacctgctgaacaaccccttcaactacgaggtggaccacatcatccccagaagcgtgtccttcgacaacagcttcaacaacaaggtgctggtgaagcaggaggaggccagcaagaagggcaaccggacccccttccagtacctgagcagcagcgacagcaagatcagctacgagaccttcaagaagcacatcctgaacctggccaagggcaagggcagaatcagcaagaccaagaaggagtacctgctggaggagcgggacatcaacaggttctccgtgcagaaggacttcatcaaccggaacctggtggacaccagatacgccaccagaggcctgatgaacctgctgcggagctacttcagagtgaacaacctggacgtgaaggtgaagtccatcaacggcggcttcaccagcttcctgcggcggaagtggaagttcaagaaggagcggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccaccgggtggacaagaagcccaacagagagctgatcaacgacaccctgtactccacccggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtactccaagaaggacaacggccccgtgatcaagaagatcaagtactacggcaacaagctgaacgcccacctggacatcaccgacgactaccccaacagcagaaacaaggtggtgaagctgtccctgaagccctacagattcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgcctccttctacaacaacgacctgatcaagatcaacggcgagctgtacagagtgatcggcgtgaacaacgacctgctgaaccggatcgaggtgaacatgatcgacatcacctaccgcgagtacctggagaacatgaacgacaagaggccccccaggatcatcaagaccatcgcctccaagacccagagcatcaagaagtacagcaccgacatcctgggcaacctgtacgaggtgaagtccaagaagcacccccagatcatcaagaagggcggcaccggcggccccaagaagaagaggaaggtgggccgggccctggaccccgagcggatcatcggcgccaccgacagcagcggcgagctgatgttcctgatgaagtggaaggacagcgacgaggccgacctggtgctggccaaggaggccaacatgaagtgcccccagatcgtgatcgccttctacgaggagcggctgacctggcacagctgccccgaggacgaggcccagtacccctacgacgtgcccgactacgcctgatatttgtgaaatttgtgatgctattgctttatttgtaaccatctagctttatttgtgaaatttgtgatgctattgctttatttgtaaccattttatttgtgaaatttgtgatgctattgctttatttgtaaccattataagctgcaataaacaagttaacaacaacaattgcattcattttatgtttcaggttcagggggagatgtgggaggttttttaaagcgggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnnngtttaagtactctgtgctggaaacagcacagaatctacttaaacaaggcaaaatgccgtgtttatctcgtcaacttgttggcgagatttttttggtaccggaccg

SEQ ID NO: 53 - DNA, 4451 bp, Artificial Sequence
  AIO CLH-23 (human CKM-HP1gamma)
  misc_feature (4326)..(4346): n is a, c, g, or t
acgcgtgatatcggacacccgagacgcccggttataattaaccaggacacgtggcgaccccccccaacacctgcccgacctctaaaaataactcctggacacccgagacgcccggttataattaaccaggacacgtggcgaccccccccaacacctgcccgacctctaaaaataactcctggacacccgagacgcccggttataattaaccaggacacgtggcgaccccccccaacacctgcccgacctctaaaaataacccctccctggggacaacccctcccagccaatagcacagcctaggtccccctatataaggccacggctgctggcccttcctcattctcagtgtcacctccaggatacagacagccccccttcagcccagcccgccaccatggcccccaagaagaagaggaaggtggaggccagcaagcggaactacatcctgggcctggccatcggcatcaccagcgtgggctacggcatcatcgactacgagacccgggacgtgatcgacgccggcgtgcggctgttcaaggaggccaacgtggagaacaacgagggcaggcggagcaagagaggcgccagaaggctgaagcggcggaggcggcacagaatccagagagtgaagaagctgctgttcgactacaacctgctgaccgaccacagcgagctgagcggcatcaacccctacgaggccagagtgaagggcctgagccagaagctgagcgaggaggagttcagcgccgccctgctgcacctggccaagagaagaggcgtgcacaacgtgaacgaggtggaggaggacaccggcaacgagctgtccaccaaggagcagatcagccggaacagcaaggccctggaggagaagtacgtggccgagctgcagctggagcggctgaagaaggacggcgaggtgcggggcagcatcaacagattcaagaccagcgactacgtgaaggaggccaagcagctgctgaaggtgcagaaggcctaccaccagctggaccagagcttcatcgacacctacatcgacctgctggagacccggcggacctactacgagggccccggcgagggcagccccttcggctggaaggacatcaaggagtggtacgagatgctgatgggccactgcacctacttccccgaggagctgcggagcgtgaagtacgcctacaacgccgacctgtacaacgccctgaacgacctgaacaacctggtgatcaccagggacgagaacgagaagctggagtactacgagaagttccagatcatcgagaacgtgttcaagcagaagaagaagcccaccctgaagcagatcgccaaggagatcctggtgaacgaggaggacatcaagggctacagagtgaccagcaccggcaagcccgagttcaccaacctgaaggtgtaccacgacatcaaggacatcaccgcccggaaggagatcatcgagaacgccgagctgctggaccagatcgccaagatcctgaccatctaccagagcagcgaggacatccaggaggagctgaccaacctgaactccgagctgacccaggaggagatcgagcagatcagcaacctgaagggctacaccggcacccacaacctgagcctgaaggccatcaacctgatcctggacgagctgtggcacaccaacgacaaccagatcgccatcttcaaccggctgaagctggtgcccaagaaggtggacctgtcccagcagaaggagatccccaccaccctggtggacgacttcatcctgagccccgtggtgaagagaagcttcatccagagcatcaaggtgatcaacgccatcatcaagaagtacggcctgcccaacgacatcatcatcgagctggcccgggagaagaactccaaggacgcccagaagatgatcaacgagatgcagaagcggaaccggcagaccaacgagcggatcgaggagatcatccggaccaccggcaaggagaacgccaagtacctgatcgagaagatcaagctgcacgacatgcaggagggcaagtgcctgtacagcctggaggccatccccctggaggacctgctgaacaaccccttcaactacgaggtggaccacatcatccccagaagcgtgtccttcgacaacagcttcaacaacaaggtgctggtgaagcaggaggaggccagcaagaagggcaaccggacccccttccagtacctgagcagcagcgacagcaagatcagctacgagaccttcaagaagcacatcctgaacctggccaagggcaagggcagaatcagcaagaccaagaaggagtacctgctggaggagcgggacatcaacaggttctccgtgcagaaggacttcatcaaccggaacctggtggacaccagatacgccaccagaggcctgatgaacctgctgcggagctacttcagagtgaacaacctggacgtgaaggtgaagtccatcaacggcggcttcaccagcttcctgcggcggaagtggaagttcaagaaggagcggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccaccgggtggacaagaagcccaacagagagctgatcaacgacaccctgtactccacccggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtactccaagaaggacaacggccccgtgatcaagaagatcaagtactacggcaacaagctgaacgcccacctggacatcaccgacgactaccccaacagcagaaacaaggtggtgaagctgtccctgaagccctacagattcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgcctccttctacaacaacgacctgatcaagatcaacggcgagctgtacagagtgatcggcgtgaacaacgacctgctgaaccggatcgaggtgaacatgatcgacatcacctaccgcgagtacctggagaacatgaacgacaagaggccccccaggatcatcaagaccatcgcctccaagacccagagcatcaagaagtacagcaccgacatcctgggcaacctgtacgaggtgaagtccaagaagcacccccagatcatcaagaagggcggcaccggcggccccaagaagaagaggaaggtgggccgggccctggaccccgagcggatcatcggcgccaccgacagcagcggcgagctgatgttcctgatgaagtggaaggacagcgacgaggccgacctggtgctggccaaggaggccaacatgaagtgcccccagatcgtgatcgccttctacgaggagcggctgacctggcacagctgccccgaggacgaggcccagtacccctacgacgtgcccgactacgcctgatatttgtgaaatttgtgatgctattgctttatttgtaaccatctagctttatttgtgaaatttgtgatgctattgctttatttgtaaccattttatttgtgaaatttgtgatgctattgctttatttgtaaccattataagctgcaataaacaagttaacaacaacaattgcattcattttatgtttcaggttcagggggagatgtgggaggttttttaaagcgggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnnngtttaagtactctgtgctggaaacagcacagaatctacttaaacaaggcaaaatgccgtgtttatctcgtcaacttgttggcgagatttttttggtaccggaccg

SEQ ID NO: 54 - DNA, 4533 bp, Artificial Sequence
  AIO CLH-26 (mouse CKM-SET)
  misc_feature (4408)..(4428): n is a, c, g, or t
acgcgtgatatcggacacccgagatgcctggttataattaacccagacatgtggctgccccccccaacacctgctgcgagctctaaaaataaccctgggacacccgagatgcctggttataattaacccagacatgtggctgccccccccaacacctgctgcgagctctaaaaataaccctgggacacccgagatgcctggttataattaacccagacatgtggctgccccccccaacacctgctgcgagctctaaaaataacccctccctggggacagcccctcctggctagtcacaccctgtaggctcctctatataacccaggggcacaggggctgccctcattctaccaccacctccacagcacagacagacactcaggagccagccagcgccaccatggcccccaagaagaagaggaaggtggaggccagcaagcggaactacatcctgggcctggccatcggcatcaccagcgtgggctacggcatcatcgactacgagacccgggacgtgatcgacgccggcgtgcggctgttcaaggaggccaacgtggagaacaacgagggcaggcggagcaagagaggcgccagaaggctgaagcggcggaggcggcacagaatccagagagtgaagaagctgctgttcgactacaacctgctgaccgaccacagcgagctgagcggcatcaacccctacgaggccagagtgaagggcctgagccagaagctgagcgaggaggagttcagcgccgccctgctgcacctggccaagagaagaggcgtgcacaacgtgaacgaggtggaggaggacaccggcaacgagctgtccaccaaggagcagatcagccggaacagcaaggccctggaggagaagtacgtggccgagctgcagctggagcggctgaagaaggacggcgaggtgcggggcagcatcaacagattcaagaccagcgactacgtgaaggaggccaagcagctgctgaaggtgcagaaggcctaccaccagctggaccagagcttcatcgacacctacatcgacctgctggagacccggcggacctactacgagggccccggcgagggcagccccttcggctggaaggacatcaaggagtggtacgagatgctgatgggccactgcacctacttccccgaggagctgcggagcgtgaagtacgcctacaacgccgacctgtacaacgccctgaacgacctgaacaacctggtgatcaccagggacgagaacgagaagctggagtactacgagaagttccagatcatcgagaacgtgttcaagcagaagaagaagcccaccctgaagcagatcgccaaggagatcctggtgaacgaggaggacatcaagggctacagagtgaccagcaccggcaagcccgagttcaccaacctgaaggtgtaccacgacatcaaggacatcaccgcccggaaggagatcatcgagaacgccgagctgctggaccagatcgccaagatcctgaccatctaccagagcagcgaggacatccaggaggagctgaccaacctgaactccgagctgacccaggaggagatcgagcagatcagcaacctgaagggctacaccggcacccacaacctgagcctgaaggccatcaacctgatcctggacgagctgtggcacaccaacgacaaccagatcgccatcttcaaccggctgaagctggtgcccaagaaggtggacctgtcccagcagaaggagatccccaccaccctggtggacgacttcatcctgagccccgtggtgaagagaagcttcatccagagcatcaaggtgatcaacgccatcatcaagaagtacggcctgcccaacgacatcatcatcgagctggcccgggagaagaactccaaggacgcccagaagatgatcaacgagatgcagaagcggaaccggcagaccaacgagcggatcgaggagatcatccggaccaccggcaaggagaacgccaagtacctgatcgagaagatcaagctgcacgacatgcaggagggcaagtgcctgtacagcctggaggccatccccctggaggacctgctgaacaaccccttcaactacgaggtggaccacatcatccccagaagcgtgtccttcgacaacagcttcaacaacaaggtgctggtgaagcaggaggaggccagcaagaagggcaaccggacccccttccagtacctgagcagcagcgacagcaagatcagctacgagaccttcaagaagcacatcctgaacctggccaagggcaagggcagaatcagcaagaccaagaaggagtacctgctggaggagcgggacatcaacaggttctccgtgcagaaggacttcatcaaccggaacctggtggacaccagatacgccaccagaggcctgatgaacctgctgcggagctacttcagagtgaacaacctggacgtgaaggtgaagtccatcaacggcggcttcaccagcttcctgcggcggaagtggaagttcaagaaggagcggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccaccgggtggacaagaagcccaacagagagctgatcaacgacaccctgtactccacccggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtactccaagaaggacaacggccccgtgatcaagaagatcaagtactacggcaacaagctgaacgcccacctggacatcaccgacgactaccccaacagcagaaacaaggtggtgaagctgtccctgaagccctacagattcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgcctccttctacaacaacgacctgatcaagatcaacggcgagctgtacagagtgatcggcgtgaacaacgacctgctgaaccggatcgaggtgaacatgatcgacatcacctaccgcgagtacctggagaacatgaacgacaagaggccccccaggatcatcaagaccatcgcctccaagacccagagcatcaagaagtacagcaccgacatcctgggcaacctgtacgaggtgaagtccaagaagcacccccagatcatcaagaagggcggcaccggcggccccaagaagaagaggaaggtgggccgggcctacgacctgtgcatcttcaggacagacgacggccggggctggggcgtgcggaccctggagaagatccggaagaacagcttcgtgatggagtacgtgggcgagatcatcaccagcgaggaggccgagcggcggggccagatctacgaccggcagggcgccacctacctgttcgacctggactacgtggaggacgtgtacaccgtggacgccgcctactacggcaacatcagccacttcgtgaaccacagctgcgaccccaacctgcaggtgtacaacgtgttcatcgacaacctggacgagcggctgccccgctacccctacgacgtgcccgactacgcctgatatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcgggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnnngtttaagtactctgtgctggaaacagcacagaatctacttaaacaaggcaaaatgccgtgtttatctcgtcaacttgttggcgagatttttttggtaccggaccg

SEQ ID NO: 55 - DNA, 4530 bp, Artificial Sequence
  AIO CLH-26 (human CKM-SET)
  misc_feature (4405)..(4425): n is a, c, g, or t
acgcgtgatatcggacacccgagacgcccggttataattaaccaggacacgtggcgaccccccccaacacctgcccgacctctaaaaataactcctggacacccgagacgcccggttataattaaccaggacacgtggcgaccccccccaacacctgcccgacctctaaaaataactcctggacacccgagacgcccggttataattaaccaggacacgtggcgaccccccccaacacctgcccgacctctaaaaataacccctccctggggacaacccctcccagccaatagcacagcctaggtccccctatataaggccacggctgctggcccttcctcattctcagtgtcacctccaggatacagacagccccccttcagcccagcccgccaccatggcccccaagaagaagaggaaggtggaggccagcaagcggaactacatcctgggcctggccatcggcatcaccagcgtgggctacggcatcatcgactacgagacccgggacgtgatcgacgccggcgtgcggctgttcaaggaggccaacgtggagaacaacgagggcaggcggagcaagagaggcgccagaaggctgaagcggcggaggcggcacagaatccagagagtgaagaagctgctgttcgactacaacctgctgaccgaccacagcgagctgagcggcatcaacccctacgaggccagagtgaagggcctgagccagaagctgagcgaggaggagttcagcgccgccctgctgcacctggccaagagaagaggcgtgcacaacgtgaacgaggtggaggaggacaccggcaacgagctgtccaccaaggagcagatcagccggaacagcaaggccctggaggagaagtacgtggccgagctgcagctggagcggctgaagaaggacggcgaggtgcggggcagcatcaacagattcaagaccagcgactacgtgaaggaggccaagcagctgctgaaggtgcagaaggcctaccaccagctggaccagagcttcatcgacacctacatcgacctgctggagacccggcggacctactacgagggccccggcgagggcagccccttcggctggaaggacatcaaggagtggtacgagatgctgatgggccactgcacctacttccccgaggagctgcggagcgtgaagtacgcctacaacgccgacctgtacaacgccctgaacgacctgaacaacctggtgatcaccagggacgagaacgagaagctggagtactacgagaagttccagatcatcgagaacgtgttcaagcagaagaagaagcccaccctgaagcagatcgccaaggagatcctggtgaacgaggaggacatcaagggctacagagtgaccagcaccggcaagcccgagttcaccaacctgaaggtgtaccacgacatcaaggacatcaccgcccggaaggagatcatcgagaacgccgagctgctggaccagatcgccaagatcctgaccatctaccagagcagcgaggacatccaggaggagctgaccaacctgaactccgagctgacccaggaggagatcgagcagatcagcaacctgaagggctacaccggcacccacaacctgagcctgaaggccatcaacctgatcctggacgagctgtggcacaccaacgacaaccagatcgccatcttcaaccggctgaagctggtgcccaagaaggtggacctgtcccagcagaaggagatccccaccaccctggtggacgacttcatcctgagccccgtggtgaagagaagcttcatccagagcatcaaggtgatcaacgccatcatcaagaagtacggcctgcccaacgacatcatcatcgagctggcccgggagaagaactccaaggacgcccagaagatgatcaacgagatgcagaagcggaaccggcagaccaacgagcggatcgaggagatcatccggaccaccggcaaggagaacgccaagtacctgatcgagaagatcaagctgcacgacatgcaggagggcaagtgcctgtacagcctggaggccatccccctggaggacctgctgaacaaccccttcaactacgaggtggaccacatcatccccagaagcgtgtccttcgacaacagcttcaacaacaaggtgctggtgaagcaggaggaggccagcaagaagggcaaccggacccccttccagtacctgagcagcagcgacagcaagatcagctacgagaccttcaagaagcacatcctgaacctggccaagggcaagggcagaatcagcaagaccaagaaggagtacctgctggaggagcgggacatcaacaggttctccgtgcagaaggacttcatcaaccggaacctggtggacaccagatacgccaccagaggcctgatgaacctgctgcggagctacttcagagtgaacaacctggacgtgaaggtgaagtccatcaacggcggcttcaccagcttcctgcggcggaagtggaagttcaagaaggagcggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccaccgggtggacaagaagcccaacagagagctgatcaacgacaccctgtactccacccggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtactccaagaaggacaacggccccgtgatcaagaagatcaagtactacggcaacaagctgaacgcccacctggacatcaccgacgactaccccaacagcagaaacaaggtggtgaagctgtccctgaagccctacagattcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgcctccttctacaacaacgacctgatcaagatcaacggcgagctgtacagagtgatcggcgtgaacaacgacctgctgaaccggatcgaggtgaacatgatcgacatcacctaccgcgagtacctggagaacatgaacgacaagaggccccccaggatcatcaagaccatcgcctccaagacccagagcatcaagaagtacagcaccgacatcctgggcaacctgtacgaggtgaagtccaagaagcacccccagatcatcaagaagggcggcaccggcggccccaagaagaagaggaaggtgggccgggcctacgacctgtgcatcttcaggacagacgacggccggggctggggcgtgcggaccctggagaagatccggaagaacagcttcgtgatggagtacgtgggcgagatcatcaccagcgaggaggccgagcggcggggccagatctacgaccggcagggcgccacctacctgttcgacctggactacgtggaggacgtgtacaccgtggacgccgcctactacggcaacatcagccacttcgtgaaccacagctgcgaccccaacctgcaggtgtacaacgtgttcatcgacaacctggacgagcggctgccccgctacccctacgacgtgcccgactacgcctgatatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcgggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgnnnnnnnnnnnnnnnnnnnnngtttaagtactctgtgctggaaacagcacagaatctacttaaacaaggcaaaatgccgtgtttatctcgtcaacttgttggcgagatttttttggtaccggaccg

SEQ ID NO: 56 - protein, 1120 aa, Artificial Sequence
  MeCP2 TRD AAV Vector
MAPKKKRKVEASKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGGTGGPKKKRKVGRAGRKPGSVVAAAAAEAKKKAVKESSIRSVQETVLPIKKRKTRA

SEQ ID NO: 57 - protein, 1149 aa, Artificial Sequence
  HP1alpha AAV Vector
MAPKKKRKVEASKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGGTGGPKKKRKVGRAEPEKIIGATDSCGDLMFLMKWKDTDEADLVLAKEANVKCPQIVIAFYEERLTWHAYPEDAENKEKETAKSA

SEQ ID NO: 58 - protein, 1142 aa, Artificial Sequence
  HP1gamma AAV Vector
MAPKKKRKVEASKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGGTGGPKKKRKVGRALDPERIIGATDSSGELMFLMKWKDSDEADLVLAKEANMKCPQIVIAFYEERLTWHSCPEDEAQA